SEQUENCE LISTING (76 sequences)

SEQ ID NO: 1 - protein, 29 aa, Artificial sequence
  sequence is synthesized
REIGGGEAGAVIGGSAGASPPSTLTPDSR

SEQ ID NO: 2 - protein, 32 aa, Artificial sequence
  sequence is synthesized
AAPLEEMEAPAADAIMSPEEELDGYEPEPLGK

SEQ ID NO: 3 - protein, 40 aa, Artificial sequence
  sequence is synthesized
RPAVLPLLELVGESGNNTSTDGSLPSTPPPAEEEEDELYR

SEQ ID NO: 4 - protein, 10 aa, Artificial sequence
  sequence is synthesized
GSAGASPPST

SEQ ID NO: 5 - protein, 10 aa, Artificial sequence
  sequence is synthesized
GSVGAEDPVT

SEQ ID NO: 6 - protein, 10 aa, Artificial sequence
  sequence is synthesized
ADAIMSPEEE

SEQ ID NO: 7 - protein, 10 aa, Artificial sequence
  sequence is synthesized
AAAIVSPEEE

SEQ ID NO: 8 - protein, 10 aa, Artificial sequence
  sequence is synthesized
TSTDGSLPST

SEQ ID NO: 9 - protein, 10 aa, Artificial sequence
  sequence is synthesized
SGADGSLPST

SEQ ID NO: 10 - protein, 6 aa, Artificial sequence
  sequence is synthesized
DGSLPS

SEQ ID NO: 11 - protein, 29 aa, Artificial sequence
  sequence is synthesized
REIGGGEAGAVIGGSAGASPPSTLTPDSR

SEQ ID NO: 12 - protein, 32 aa, Artificial sequence; identical to SEQ ID NO: 2
  sequence is synthesized

SEQ ID NO: 13 - protein, 40 aa, Artificial sequence; identical to SEQ ID NO: 3
  sequence is synthesized

SEQ ID NO: 14 - protein, 20 aa, Artificial sequence
  sequence is synthesized
VARPPPIGAEVPDVTATPAR

SEQ ID NO: 15 - DNA, 20 bp, Artificial sequence
  sequence is synthesized
ccatgtggtgagtggatctc

SEQ ID NO: 16 - DNA, 20 bp, Artificial sequence
  sequence is synthesized
ctgcattcccagagacaaga

SEQ ID NO: 17 - DNA, 23 bp, Artificial sequence
  sequence is synthesized
tccgtgtttgggatgtggagaca

SEQ ID NO: 18 - DNA, 19 bp, Artificial sequence
  sequence is synthesized
agcggattctcatggaaca

SEQ ID NO: 19 - DNA, 18 bp, Artificial sequence
  sequence is synthesized
ctggtcagccaggagctt

SEQ ID NO: 20 - DNA, 24 bp, Artificial sequence
  sequence is synthesized
tccacaagctgaaggcagacaagg

SEQ ID NO: 21 - DNA, 20 bp, Artificial sequence
  sequence is synthesized
cataactgctctgccagctc

SEQ ID NO: 22 - DNA, 20 bp, Artificial sequence
  sequence is synthesized
ggtcactcggtaccattcct

SEQ ID NO: 23 - DNA, 25 bp, Artificial sequence
  sequence is synthesized
tggatgccaaatcactatgtgctgc

SEQ ID NO: 24 - DNA, 20 bp, Artificial sequence
  sequence is synthesized
ggatgggtttgtggagttct

SEQ ID NO: 25 - DNA, 20 bp, Artificial sequence
  sequence is synthesized
tcctactccagcaacacctg

SEQ ID NO: 26 - DNA, 22 bp, Artificial sequence
  sequence is synthesized
tggcatcaggaatgtgctgctg

SEQ ID NO: 27 - RNA, 21 nt, Artificial sequence
  sequence is synthesized
guggaauuuguggaacaucuu

SEQ ID NO: 28 - RNA, 23 nt, Artificial sequence
  sequence is synthesized
ccuucucuggagagagaaauguu

SEQ ID NO: 29 - RNA, 19 nt, Artificial sequence
  sequence is synthesized
gaccggauguuaaccuuua

SEQ ID NO: 30 - RNA, 19 nt, Artificial sequence
  sequence is synthesized
ccugcgaccuuaagauuug

SEQ ID NO: 31 - RNA, 19 nt, Artificial sequence
  sequence is synthesized
ccaauaaacggaucacagu

SEQ ID NO: 32 - RNA, 19 nt, Artificial sequence
  sequence is synthesized
agacugaccuguacaaguu

SEQ ID NO: 33 - RNA, 19 nt, Artificial sequence
  sequence is synthesized
ucgaguagcuaucaagaaa

SEQ ID NO: 34 - RNA, 19 nt, Artificial sequence
  sequence is synthesized
caccaaccaucgagcaaau

SEQ ID NO: 35 - RNA, 19 nt, Artificial sequence
  sequence is synthesized
ggugugcucugcuuaugau

SEQ ID NO: 36 - RNA, 19 nt, Artificial sequence
  sequence is synthesized
acaccaaccucucguacau

SEQ ID NO: 37 - RNA, 19 nt, Artificial sequence
  sequence is synthesized
gcccaguaauauaguagua

SEQ ID NO: 38 - RNA, 19 nt, Artificial sequence
  sequence is synthesized
ggcaugggcuacaaggaaa

SEQ ID NO: 39 - RNA, 19 nt, Artificial sequence
  sequence is synthesized
gaauaguaugcgcagcuua

SEQ ID NO: 40 - RNA, 19 nt, Artificial sequence
  sequence is synthesized
gaugacgccuuauguagug

SEQ ID NO: 41 - RNA, 19 nt, Artificial sequence
  sequence is synthesized
gauuguuugugcugcauuu

SEQ ID NO: 42 - RNA, 19 nt, Artificial sequence
  sequence is synthesized
ggcugucgaugauagguua

SEQ ID NO: 43 - RNA, 19 nt, Artificial sequence
  sequence is synthesized
agccaacugugaggaauua

SEQ ID NO: 44 - RNA, 19 nt, Artificial sequence
  sequence is synthesized
ucgugaacuuguccucuua

SEQ ID NO: 45 - RNA, 19 nt, Artificial sequence
  sequence is synthesized
cauauguggugacacguua

SEQ ID NO: 46 - RNA, 19 nt, Artificial sequence
  sequence is synthesized
ggacgacgccuuacagcau

SEQ ID NO: 47 - RNA, 19 nt, Artificial sequence
  sequence is synthesized
ggaauuagaccaugagcga

SEQ ID NO: 48 - RNA, 19 nt, Artificial sequence
  sequence is synthesized
ggaaagaacuuaucuacaa

SEQ ID NO: 49 - RNA, 19 nt, Artificial sequence
  sequence is synthesized
cgacgagcacguucaauuc

SEQ ID NO: 50 - RNA, 19 nt, Artificial sequence
  sequence is synthesized
ccauagaccuccuuggaag

SEQ ID NO: 51 - RNA, 19 nt, Artificial sequence
  sequence is synthesized
gcccugagguucuggcaaa

SEQ ID NO: 52 - RNA, 19 nt, Artificial sequence
  sequence is synthesized
acguucaauuccugguuua

SEQ ID NO: 53 - RNA, 19 nt, Artificial sequence
  sequence is synthesized
gaagcguguuacuuacaaa

SEQ ID NO: 54 - RNA, 19 nt, Artificial sequence
  sequence is synthesized
gcgcuaagguggccaucaa

SEQ ID NO: 55 - RNA, 19 nt, Artificial sequence
  sequence is synthesized
gcaagacgcuguucaaggg

SEQ ID NO: 56 - RNA, 19 nt, Artificial sequence
  sequence is synthesized
ggagacgccucugugaaga

SEQ ID NO: 57 - RNA, 19 nt, Artificial sequence
  sequence is synthesized
ucaaaggccuuaaguacau

SEQ ID NO: 58 - RNA, 19 nt, Artificial sequence
  sequence is synthesized
gccguuugaugauuccuua

SEQ ID NO: 59 - RNA, 19 nt, Artificial sequence
  sequence is synthesized
gcucaaaggccuuaaguac

SEQ ID NO: 60 - RNA, 19 nt, Artificial sequence
  sequence is synthesized
ggaguggcaugaagcugua

SEQ ID NO: 61 - RNA, 19 nt, Artificial sequence
  sequence is synthesized
caaggucucuggaggaauu

SEQ ID NO: 62 - RNA, 19 nt, Artificial sequence
  sequence is synthesized
gucagaagcuuacagauga

SEQ ID NO: 63 - RNA, 19 nt, Artificial sequence
  sequence is synthesized
guccaucauucaugcgaaa

SEQ ID NO: 64 - RNA, 19 nt, Artificial sequence
  sequence is synthesized
cuacagagaacugcgguua

SEQ ID NO: 65 - RNA, 20 nt, Artificial sequence
  sequence is synthesized
gaucuggagcucucgguucu

SEQ ID NO: 66 - RNA, 19 nt, Artificial sequence
  sequence is synthesized
gcaaagaaauggauaucaa

SEQ ID NO: 67 - RNA, 19 nt, Artificial sequence
  sequence is synthesized
ggaagaggcuaaaugucua

SEQ ID NO: 68 - DNA, 21 bp, Artificial sequence
  sequence is synthesized
cgaaaugacuauuaccugatt

SEQ ID NO: 69 - RNA, 21 nt, Artificial sequence
  sequence is synthesized
ucagguaauagucauuucgga

SEQ ID NO: 70 - protein, 40 aa, Artificial sequence
  sequence is synthesized
RPAVLPLLELVGESGNNTSTDGPSLPSPTPPPAEEEEDEL

SEQ ID NO: 71 - protein, 40 aa, Artificial sequence; identical to SEQ ID NO: 3
  sequence is synthesized

SEQ ID NO: 72 - protein, 40 aa, Artificial sequence; identical to SEQ ID NO: 3
  sequence is synthesized

SEQ ID NO: 73 - protein, 40 aa, Artificial sequence; identical to SEQ ID NO: 3
  sequence is synthesized

SEQ ID NO: 74 - protein, 40 aa, Artificial sequence; identical to SEQ ID NO: 3
  sequence is synthesized

SEQ ID NO: 75 - protein, 32 aa, Artificial sequence; identical to SEQ ID NO: 2
  sequence is synthesized

SEQ ID NO: 76 - protein, 32 aa, Artificial sequence; identical to SEQ ID NO: 2
  sequence is synthesized

(19) United States
(12) Patent Application Publication
(10) Pub. No.: US 2016/0136295 A1
(43) Pub. Date: May 19, 2016

(54) BIOMARKERS FOR TREATMENT WITH ANTI-TUBULIN CHEMOTHERAPEUTIC COMPOUNDS

(71) Applicant: GENENTECH, INC., South San Francisco, CA (US)

(72) Inventor: Ingrid Wertz, Redwood City, CA (US)

(73) Assignee: GENENTECH, INC., South San Francisco, CA (US)

(21) Appl. No.: 14/322,065

(22) Filed: Jul. 2, 2014

Related U.S. Application Data
(63) Continuation of application No. PCT/US2012/027446, filed on Mar. 2, 2012.

(51) Int. Cl. A61K 47/48; C12Q 1/68; G01N 33/68; A61K 38/05

(52) U.S. Cl. CPC A61K 47/48384; A61K 38/05; C12Q 1/6886; G01N 33/6893; C12Q 2600/106; C12Q 2600/156; G01N 2800/52; G01N 2800/7023

(57) ABSTRACT

Provided herein are biomarkers for predicting sensitivity to treating cancer with anti-tubulin chemotherapeutic agents.

### CROSS REFERENCE TO RELATED APPLICATIONS

This application is a continuation application filed under 37 CFR §1.53(b) and claims the benefit of priority under 35 USC §119 and §365 of PCT Application No. PCT/US2012/027446 filed on 2 Mar. 2012, which is incorporated by reference in entirety.

### FIELD OF THE INVENTION

The invention relates generally to selection and treatment of patients with hyperproliferative disorders such as cancer with anti-tubulin chemotherapeutic compounds. The invention also relates to methods of using biomarkers for in vitro, in situ, and in vivo diagnosis or treatment of hyperproliferative disorders.

### BACKGROUND OF THE INVENTION

Microtubules play pivotal roles in fundamental cellular processes and are targets of anti-tubulin chemotherapeutics (Jackson et al (2007) Nat. Rev. Cancer 7(2):107-117). Microtubule-targeted agents such as paclitaxel and vincristine are prescribed widely for various malignancies including ovarian and breast adenocarcinomas, non-small cell lung cancer (NSCLC), leukemias, and lymphomas. These agents arrest cells in mitosis and subsequently induce cell death via poorly-defined mechanisms (Rieder, C. L. and Maiato, H. (2004) Developmental Cell 7:637-651). The strategies that resistant tumor cells employ to evade killing by anti-tubulin agents are also unclear. Anti-tubulin chemotherapeutics are approved for multiple indications including breast, lung, and ovarian solid tumors, and hematological malignancies, including lymphoma and leukemias (Jackson et al (2007) Nat. Rev. Cancer 7(2):107-117).

Measuring expression levels of biomarkers (e.g., secreted proteins in plasma) can be an effective means to identify patients and patient populations that will respond to specific therapies including, e.g., treatment with chemotherapeutic agents. There is a need for more effective means for determining which patients with hyperproliferative disorders such as cancer will respond to which treatment with chemotherapeutic agents, and for incorporating such determinations into more effective treatment regimens for patients, whether the chemotherapeutic agents are used as single agents or combined with other agents.

Bcl-2 family proteins are key regulators of cell survival and can either promote or inhibit cell death (Youle, R. J. and Strasser, A. (2008) Nat Rev Mol Cell Biol 9:47-59). Pro-survival members, including Bcl-X_{L} and Mcl-1, inhibit apoptosis by blocking the death mediators Bax and Bak. Uninhibited Bax and Bak permeabilize outer mitochondrial membranes and release proapoptotic factors that activate caspases, the proteases that catalyze cellular demise. This intrinsic, or mitochondrial, pathway is initiated by the damage-sensing BH3-only proteins including Bim and Noxa that neutralize the pro-survival family members when cells are irreparably damaged (Willis, S. N. et al. (2007) Science (New York, N.Y 315:856-859). Pro-survival members, particularly Bcl-2, Bcl-xL and Mcl-1 are over-expressed in hematopoietic and solid tumors and facilitate chemotherapeutic resistance (Youle et al (2008) Nature Rev. Mol. Cell Biol. 9(1):47-59). Bcl-2 is a clinically validated drug target in hematological malignancies. Small molecule BH3 mimetics ABT-263, navitoclax, a dual Bcl-2/Bcl-xL inhibitor (Oltersdorf et al (2005) Nature 435:677; Petros et al (2006) J. Med. Chem. 49:656; Wendt et al (2006) J. Med. Chem. 49:1165; Bruncko et al (2007) J. Med. Chem. 50:641; Tan et al (2011) Clin Cancer Res. March 15; 17(6):1394-404. Epub 2011 Jan. 10; U.S. Pat. No. 7,767,684; U.S. Pat. No. 7,390,799), and ABT-199, a Bcl-2 selective inhibitor (US 2010/0305122), are in clinical trials.

Antibody-drug conjugates (ADC) are targeted chemotherapeutic molecules which combine ideal properties of both antibodies and cytotoxic drugs by targeting potent cytotoxic drugs to antigen-expressing tumor cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. Effective ADC development for a given target antigen depends on optimization of parameters such as target antigen expression levels, tumor accessibility (Kovtun, Y. V. and Goldmacher V. S. (2007) Cancer Letters 255:232-240), antibody selection (U.S. Pat. No. 7,964,566), linker stability (Erickson et al (2006) Cancer Res. 66(8):4426-4433; Doronina et al (2006) Bioconjugate Chem. 17:114-124; Alley et al (2008) Bioconjugate Chem. 19:759-765), cytotoxic drug mechanism of action and potency, drug loading (Hamblett et al (2004) Clin. Cancer Res. 10:7063-7070) and mode of linker-drug conjugation to the antibody (Lyon, R. et al (2012) Methods in Enzym. 502:123-138; Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614). Antibody-drug conjugates with anti-tubulin drug moieties have been developed for treatment of cancer (Doronina et al (2003) Nature Biotechnology 21(7):778-784; Lewis Phillips, et al (2008) Cancer Res. 68:9280-9290

### SUMMARY OF THE INVENTION

In one aspect the invention includes a method of treating a hyperproliferative disorder in a patient comprising administering a therapeutically effective amount of an anti-tubulin chemotherapeutic agent to the patient, wherein a biological sample obtained from the patient, prior to administration of the anti-tubulin chemotherapeutic agent to the patient, has been tested for Mcl-1 and/or FBW7 status, and wherein Mcl-1 and/or FBW7 status is indicative of therapeutic responsiveness by the patient to the anti-tubulin chemotherapeutic agent. In one embodiment, the biological sample has been tested by measuring functional Mcl-1 protein level, wherein an increased level of functional Mcl-1 protein indicates that the patient will be resistant to the anti-tubulin chemotherapeutic agent. In another embodiment, the biological sample has been tested by measuring functional FBW7 protein level, wherein a decreased level of functional FBW7 protein indicates that the patient will be resistant to the anti-tubulin chemotherapeutic agent.

In one aspect the invention includes a method of monitoring whether a patient with a hyperproliferative disorder will respond to treatment with an anti-tubulin chemotherapeutic agent, the method comprising:

(a) detecting Mcl-1 and/or FBW7 in a biological sample obtained from the patient following administration of the at least one dose of an anti-tubulin chemotherapeutic agent; and

(b) comparing Mcl-1 and/or FBW7 status in a biological sample obtained from the patient prior to administration of the anti-tubulin chemotherapeutic agent to the patient,

wherein a change or modulation of Mcl-1 and/or FBW7 status in the sample obtained following administration of the anti-tubulin chemotherapeutic agent identifies a patient who will respond to treatment with an anti-tubulin chemotherapeutic agent.

In one aspect the invention includes a method of optimizing therapeutic efficacy of an anti-tubulin chemotherapeutic agent, the method comprising:

(a) detecting Mcl-1 and/or FBW7 in a biological sample obtained from a patient who has received at least one dose of an anti-tubulin chemotherapeutic agent following administration of the at least one dose of an anti-tubulin chemotherapeutic agent; and

(b) comparing the Mcl-1 and/or FBW7 status in a biological sample obtained from the patient prior to administration of the anti-tubulin chemotherapeutic agent to the patient,

wherein a change or modulation of Mcl-1 and/or FBW7 in the sample obtained following administration of the anti-tubulin chemotherapeutic agent identifies a patient who has an increased likelihood of benefit from treatment with an anti-tubulin chemotherapeutic agent.

The anti-tubulin chemotherapeutic agent is selected from paclitaxel, docetaxel, vincristine, vinblastine, vinorelbine, eribulin, combretastatin, maytansines, dolastatins, auristatins, and the antibody-drug conjugates thereof.

A change in Mcl-1 or FBW7 levels or activity can be used as a pharmacodynamic biomarker (“PD biomarkers”) for the therapeutic effects of anti-tubulin chemotherapeutic agents.

In certain embodiments, the proper dosage of anti-tubulin chemotherapeutic agents can be determined and adjusted based upon, inhibition or modulation of signaling pathway, using PD biomarkers Mcl-1 or FBW7.

In one aspect the invention includes a identifying a biomarker for monitoring responsiveness to an anti-tubulin chemotherapeutic agent, the method comprising:

(a) detecting the expression, modulation, or activity of a biomarker in a biological sample obtained from a patient who has received at least one dose of an anti-tubulin chemotherapeutic agent wherein the biomarker is Mcl-1 and/or FBW7; and

(b) comparing the expression, modulation, or activity of the biomarker to the status of the biomarker in a reference sample wherein the reference sample is a biological sample obtained from the patient prior to administration of the anti-tubulin chemotherapeutic agent to the patient;

wherein the modulation of the biomarker changes by at least 2 fold lower compared to the reference sample is identified as a biomarker useful for monitoring responsiveness to an anti-tubulin chemotherapeutic agent.

In one aspect the invention includes a method of treating a hyperproliferative disorder in a patient, comprising administering a therapeutically effective amount of an anti-tubulin chemotherapeutic agent the patient, wherein treatment is based upon a sample from the patient having an Mcl-1 or FBW7 mutation.

In one aspect the invention includes the use of an anti-tubulin chemotherapeutic agent in treating a hyperproliferative disorder in a patient comprising:

administering a therapeutically effective amount of an anti-tubulin chemotherapeutic agent to the patient,

wherein a biological sample obtained from the patient, prior to administration of the anti-tubulin chemotherapeutic agent to the patient, has been tested for Mcl-1 or FBW7 status, and wherein Mcl-1 or FBW7 status is indicative of therapeutic responsiveness by the patient to the anti-tubulin chemotherapeutic agent.

### BRIEF DESCRIPTION OF THE DRAWINGS

FIGS. 1(A-D) show Bcl-2 family proteins regulate cell death induced by anti-tubulin chemotherapeutic agents. Viability of cell lines treated 48 hours with indicated agents (data are presented as the mean±SEM, n=3). BAX^{-/-}/BAK^{-/-} MEFs (A) and FDM cells (B) are resistant to antimitotic-induced cell death. (C) Genetic deletion of MCL-1 and BCL-X enhances sensitivity to paclitaxel (TAXOL®). (D) Genetic deletion of MCL-1 but not BCL-X enhances sensitivity to vincristine.

FIG. 1E shows assessment of Bcl-2 family protein levels in mitotic arrest. The mitotic time course indicates when synchronized cells were collected relative to the onset of mitotic arrest: i.e. −2 is 2 hours prior to mitosis (M) and +3 is 3 hours after cells entered mitosis. CDC27 and tubulin are indicators of mitotic arrest and equal loading, respectively. cdc27-P=phosphorylated cdc27.

FIGS. 2(A-F) show SCF^{FBW7} targets Mcl-1 for proteasomal degradation in mitotic arrest. (A) MCL-1 message is not significantly decreased relative to Mcl-1 protein in mitotic arrest. (B) MG132 stabilizes Mcl-1 degradation in mitotic arrest. (C) RNAi of FBW7, but not beta (β)-TrCP, attenuates Mcl-1 degradation in mitotic arrest in HCT116 cells. (D) Mcl-1 degradation is attenuated in FBW7^{-/-} cells in mitotic arrest. Complementation with FBW7-alpha or -beta isoforms restores Mcl-1 degradation. (E) FBW7 recruits Mcl-1 to the CUL1 ubiquitin ligase complex in mitotic arrest. (F) Reconstitution of the SCF^{FBW7} ubiquitin ligase complex promotes Mcl-1 ubiquitination in vitro. Lower panel: endogenous ROC1 does not associate with dominant negative (DN) HA-CUL1.

FIGS. 3(A-G) show identification of Mcl-1 degrons and kinases that direct recruitment to FBW7 in mitotic arrest. (A) The FBW7 degron consensus, corresponding Mcl-1 residues, and mitotic phosphorylation sites are indicated on the peptides (also see FIG. S16(A-E)). Mcl-1 phosphomutant nomenclature is also indicated. (B) Association of FLAG-FBW7 with myc-Mcl-1 mutants S121A/E125A and S159A/T163A is attenuated in mitotic arrest. (C) Mcl-1 phosphomutants S121A/E125A and S159A/T163A have attenuated degradation in mitotic arrest. (D) Schematic representation of Mcl-1 or cyclin E peptides and their calculated dissociation constants (K_{d}) for FBW7 binding. (E) The Mcl-1 peptide containing the phosphorylated S121/E125 degron preferentially binds FBW7 in vitro. (F) Pharmacologic inhibition of JNK, p38, or cdk1 attenuates recruitment of myc-Mcl-1 to FLAG-FBW7 in mitotic arrest (also see FIG. S25). (G) In vitro phosphorylation of recombinant Mcl-1 drives FBW7 binding.

SEQ ID NO: 1

REIGGGEAGAVIGGSAGASPPSTLTPDSR

SEQ ID NO: 2

AAPLEEMEAPAADAIMSPEEELDGYEPEPLGK

SEQ ID NO: 3

RPAVLPLLELVGESGNNTSTDGSLPSTPPPAEEEEDELYR

FIGS. 4(A-E) show FBW7 inactivation and elevated Mcl-1 promote antimitotic resistance and tumorigenesis in human cancers. (A) FBW7-wild-type ovarian cancer cell lines that undergo mitotic arrest are sensitive to Taxol and rapidly degrade Mcl-1 relative to FBW7-mutant and Taxol-insensitive cells. FBW7 status is specified in parentheses. (B) Sensitivity to vincristine-induced death is restored in FBW7^{-/-} cells upon Mcl-1 ablation (data are presented as the mean±SEM, n=3). (C) Mcl-1 expression modulates polyploidy in FBW7-deficient cells. The percentage of cells with >4N chromosomes is indicated. (D) Mcl-1 expression accelerates mitotic slippage and attenuates apoptosis in FBW7-deficient cells. p-values: *p<0.05; ** p<0.001 (one-tailed Fisher's exact test). (E) Mcl-1 levels are elevated in NSCLC samples with mutant FBW7 or low FBW7 copy number relative to FBW7-wild-type tumors and normal lung samples (Supplementary Table 2). NSCLC FBW7-mutant samples 3 and 5 (green) also have low FBW7 copy number.

FIG. 5 shows MMAE is a synthetic, anti-tubulin agent that promotes mitotic arrest and subsequent Mcl-1 degradation in Granta-519, HCT-116 and HeLa cells. M=mitosis as indicated by phospho-cdc27; −4=4 h prior to mitosis; +2=2 h after onset of mitotic arrest.

FIG. 6A shows the anti-tubulin antibody-drug conjugate, anti-NaPi3b-MC-vc-PAB-MMAE (ADC-MMAE) promotes mitotic arrest in OVCAR3×2.1 ovarian cancer cells, relative to a negative control, (anti-gD (glycoproteins D) ADC), a non-specific binding antibody-drug conjugate.

FIG. 6B shows levels of Mcl-1, Bim, non-pBcl-xL ser62, and phospho-histone 3 in OVCAR3×2.1 ovarian cancer cells after treatment with anti-NaPi3b-MC-vc-PAB-MMAE (ADC-MMAE) relative to negative control, non-specific binding antibody-drug conjugate (anti-gD ADC)

FIG. 7A shows the anti-tubulin antibody-drug conjugate, anti-STEAP1-MC-vc-PAB-MMAE (ADC-MMAE) promotes mitotic arrest in LNCaP prostate cancer cells, relative to a negative control, (anti-gD ADC), a non-specific binding antibody-drug conjugate.

FIG. 7B shows levels of Mcl-1, Bim, non-pBcl-xL ser62, and phospho-histone 3 in LNCaP prostate cancer cells after treatment with anti-STEAP1-MC-vc-PAB-MMAE (ADC-MMAE) relative to negative control, non-specific binding antibody-drug conjugate (anti-gD ADC)

FIG. 8A shows the anti-tubulin antibody-drug conjugate, anti-STEAP1-MC-vc-PAB-MMAE (ADC-MMAE) promotes mitotic arrest in 293 cells expressing STEAP1, relative to a negative control, (anti-gD ADC), a non-specific binding antibody-drug conjugate.

FIG. 8B shows levels of Mcl-1, Bim, non-pBcl-xL ser62, and phospho-histone 3 in 293 cells expressing STEAP1 after treatment with anti-STEAP1-MC-vc-PAB-MMAE (ADC-MMAE) relative to negative control, non-specific binding antibody-drug conjugate (anti-gD ADC)

FIG. 9A shows the anti-tubulin antibody-drug conjugate, anti-ETBR-MC-vc-PAB-MMAE (ADC-MMAE) promotes mitotic arrest in UACC-257×2.2 melanoma cancer cells, relative to a negative control, (anti-gD ADC), a non-specific binding antibody-drug conjugate.

FIG. 9B shows levels of Mcl-1, Bim, non-pBcl-xL ser62, and phospho-histone 3 in UACC-257×2.2 melanoma cancer cells after treatment with anti-ETBR-MC-vc-PAB-MMAE (ADC-MMAE) relative to negative control, non-specific binding antibody-drug conjugate (anti-gD ADC)

FIG. 10A shows the anti-tubulin antibody-drug conjugate, anti-CD22-MC-vc-PAB-MMAE (ADC-MMAE) promotes mitotic arrest in Granta-519 B-cell lymphoma cancer cells, relative to a negative control, (anti-gD ADC), a non-specific binding antibody-drug conjugate.

FIG. 10B shows levels of Mcl-1, phospho-histone 3, and pBcl-xL in Granta-519 B-cell lymphoma cancer cells after treatment with anti-CD22-MC-vc-PAB-MMAE (ADC-MMAE) relative to negative control, non-specific binding antibody-drug conjugate (anti-gD ADC)

FIG. 11A shows the anti-tubulin antibody-drug conjugate, anti-CD22-MC-vc-PAB-MMAE (ADC-MMAE) promotes mitotic arrest in WSU-DLCL2 B-cell lymphoma cancer cells, relative to a negative control, (anti-gD ADC), a non-specific binding antibody-drug conjugate.

FIG. 11B shows levels of Mcl-1, phospho-histone 3, and pBcl-xL in WSU-DLCL2 B-cell lymphoma cancer cells after treatment with anti-CD22-MC-vc-PAB-MMAE (ADC-MMAE) relative to negative control, non-specific binding antibody-drug conjugate (anti-gD ADC)

FIG. 12A shows the anti-tubulin antibody-drug conjugate, anti-FcRH5-MC-vc-PAB-MMAE (ADC-MMAE) promotes mitotic arrest in EJM cells expressing FcRH5 multiple myeloma cancer cells, relative to a negative control, (anti-gD ADC), a non-specific binding antibody-drug conjugate.

FIG. 12B shows levels of Mcl-1, phospho-histone 3, and pBcl-xL in EJM cells expressing FcRH5 multiple myeloma cancer cells after treatment with anti-FcRH5-MC-vc-PAB-MMAE (ADC-MMAE) relative to negative control, non-specific binding antibody-drug conjugate (anti-gD ADC)

FIG. 13A shows the anti-tubulin antibody-drug conjugate, anti-FcRH5-MC-vc-PAB-MMAE (ADC-MMAE) promotes mitotic arrest in OPM2 cells expressing FcRH5 multiple myeloma cancer cells, relative to a negative control, (anti-gD ADC), a non-specific binding antibody-drug conjugate.

FIG. 13B shows levels of Mcl-1, phospho-histone 3, and pBcl-xL in OPM2 cells expressing FcRH5 multiple myeloma cancer cells after treatment with anti-FcRH5-MC-vc-PAB-MMAE (ADC-MMAE) relative to negative control, non-specific binding antibody-drug conjugate (anti-gD ADC)

FIG. 14 shows the anti-tubulin antibody-drug conjugate, anti-CD79b-MC-vc-PAB-MMAE (ADC-MMAE) promotes mitotic arrest and Bcl family protein modulation in Granta-519 and WSU-DLCL2 NHL B-cell lymphoma cell lines, relative to a negative, non-specific binding antibody-drug conjugate control, anti-CD22 ADC.

FIG. S1 shows a schematic illustrating the concerted activities of the phosphatases, kinases, and the SCF-FBW7 ubiquitin ligase in regulating Mcl-1 degradation in prolonged mitotic arrest.

FIGS. S2(A-E) show multiple lineages of BAX−/−/BAK−/− murine embryonic fibroblasts (MEFs) are resistant to anti-tubulin agent-induced death. Cell viability of wild-type (WT) or Bax−/−/Bak−/− MEF cell lines treated 48 hours with various doses of the indicated anti-tubulin agent drug. Data are presented as the mean±SEM, n=3.

FIG. S3 shows ablation of IAP family proteins does not enhance cell sensitivity to paclitaxel. Cell viability of MEF cell lines deficient in the indicated genes and transfected with the indicated siRNA oligos after 48 hours of treatment with various doses of paclitaxel. Note: basal levels of endogenous cIAP2 are not detectable with available antibodies.

FIG. S4 shows assessment of Bcl-2 family protein levels in mitotic arrest. HeLa cells were synchronized and released into nocodazole or paclitaxel and collected at the indicated time points. The mitotic time course follows the progression of cells in mitotic arrest: i.e. −2 is 2 hours prior to mitosis (M) and +3 is 3 hours after cells enter mitosis. cdc27-P, phosphorylated cdc27.

FIG. S5 shows Mcl-1 protein levels decrease in mitotic arrest in unsynchronized cells. HEK293T or HeLa cells were treated for 16 hours with 40 ng/mL nocodazole or 3 μg/mL aphidicolin and processed for western blot analysis as indicated.

FIG. S6 shows MG132 stabilizes Mcl-1 degradation in mitotic arrest. HCT116 cells were synchronized, released into paclitaxel, and MG132 was added as indicated when cells entered mitotic arrest. Cells were collected at the indicated time points and analyzed as indicated.

FIG. S7 shows Mcl-1 is ubiquitinated in mitotic arrest. Synchronized HeLa cells were lysed in 6M urea to dissociate non-covalently bound proteins and Mcl-1 was immunoprecipitated from lysates and blotted for ubiquitin. Mcl-1-Ub, ubiquitinated Mcl-1.

FIG. S8 shows alignment of potential Mcl-1 degrons for recruitment to FBW7 or beta-TrCP. The FBW7 or beta-TrCP degron consensus sequences are above, and alignments of human and murine Mcl-1 sequences are below.

SEQ ID NO: 4

GSAGASPPST

SEQ ID NO: 5

GSVGAEDPVT

SEQ ID NO: 6

ADAIMSPEEE

SEQ ID NO: 7

AAAIVSPEEE

SEQ ID NO: 8

TSTDGSLPST

SEQ ID NO: 9

SGADGSLPST

SEQ ID NO: 10

DGSLPS

FIG. S9 shows dominant negative CUL1 (DN-CUL1) blocks degradation of Mcl-1 in mitotic arrest. HCT116 cells were transfected with HA-DN-CUL1 or vector control, synchronized, released into paclitaxel, and collected at the indicated time points.

FIGS. S10(A-C) show the Mcl-1 ubiquitin ligase MULE does not significantly regulate Mcl-1 turnover in mitotic arrest in the evaluated cell lines. The indicated cell lines were transfected with non-specific scramble or MULE-targeting siRNA oligos, synchronized, released into paclitaxel, and collected at the indicated time points. Autoradiography bands were quantitated and normalized relative to Mcl-1 levels in the initial time point. Graphical summaries of the quantitated data are indicated below the autoradiograms.

FIG. S11 shows RNAi of FBW7 attenuates Mcl-1 degradation in mitotic arrest. The message of the indicated F-box proteins in HCT116 cells transfected with the respective siRNA oligos was measured relative to cells transfected with scramble siRNA oligo control.

FIG. S12 shows RNAi of FBW7, but not beta-TrCP, attenuates Mcl-1 degradation in mitotic arrest. HeLa cells were transfected with the indicated siRNA oligonucleotides, synchronized, released into Paclitaxel, and collected at the indicated time points. The remaining message of the indicated F-box proteins from cells transfected with the respective siRNA oligos was measured relative to cells transfected with scramble siRNA oligo control.

FIGS. S13(A-B) show FBW7 regulates Mcl-1 turnover in mitotic arrest in non-transformed cells. The indicated cell lines were transfected with non-specific scramble or FBW7-targeting siRNA oligos, synchronized, released into paclitaxel, and collected at the indicated time points. The remaining FBW7 message from cells transfected with the respective siRNA oligos was measured relative to cells transfected with scramble siRNA oligo control.

FIG. S14 shows Mcl-1 protein turnover is attenuated in mitotic arrest in FBW7−/− cells relative to wild-type parental cell lines. DLD1 or HCT116 cells were synchronized, released into paclitaxel, metabolically labeled with ³⁵S Cys/Met, and collected at the indicated time points after entry into mitotic arrest (T=0). Mcl-1 was immunoprecipitated from cell lysates and immunocomplexes were separated on SDS-PAGE gels, transferred to membranes, and exposed to film. A=Asynchronous cells.

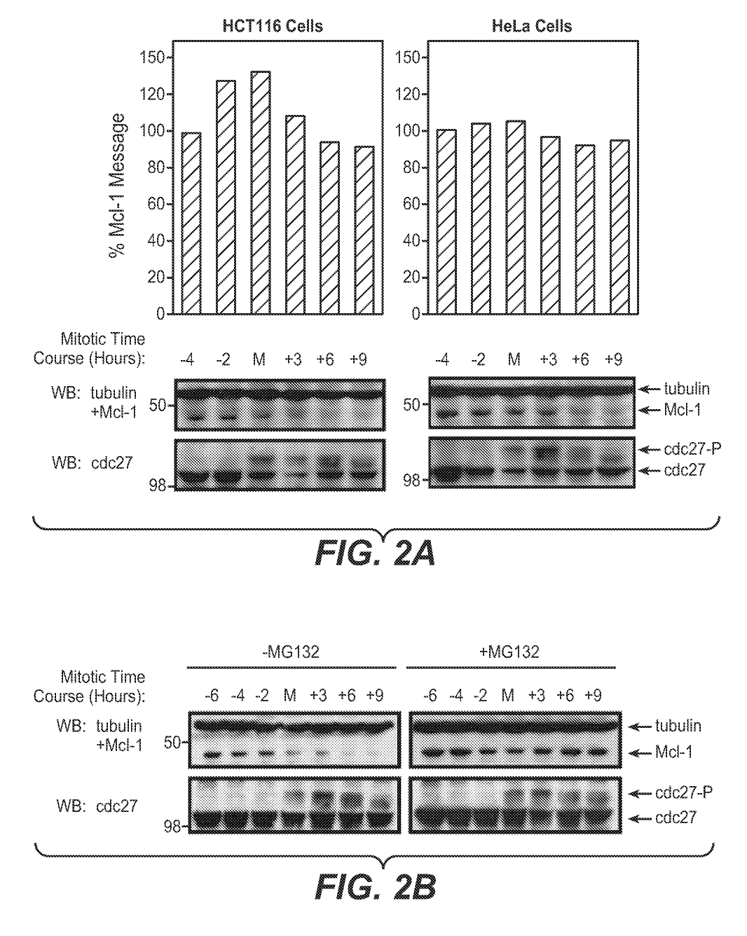

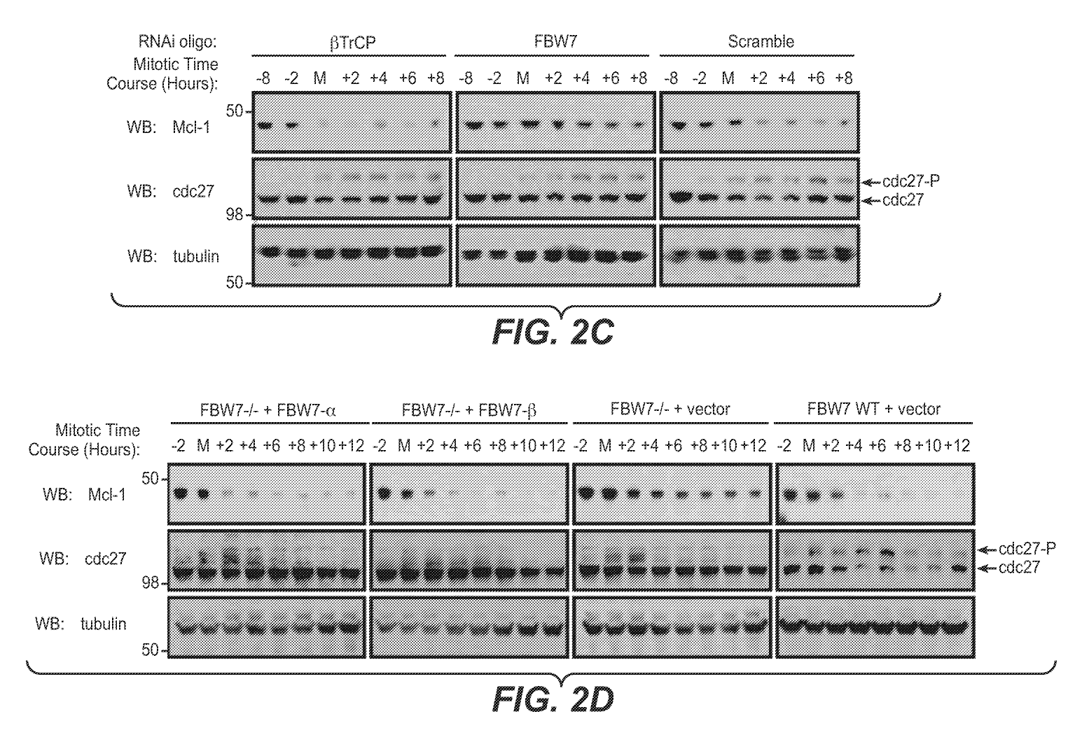

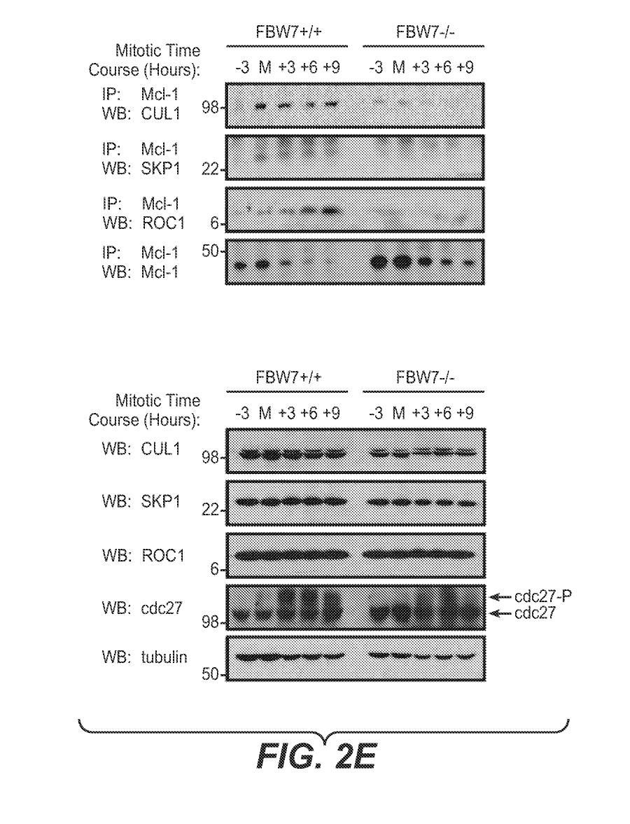

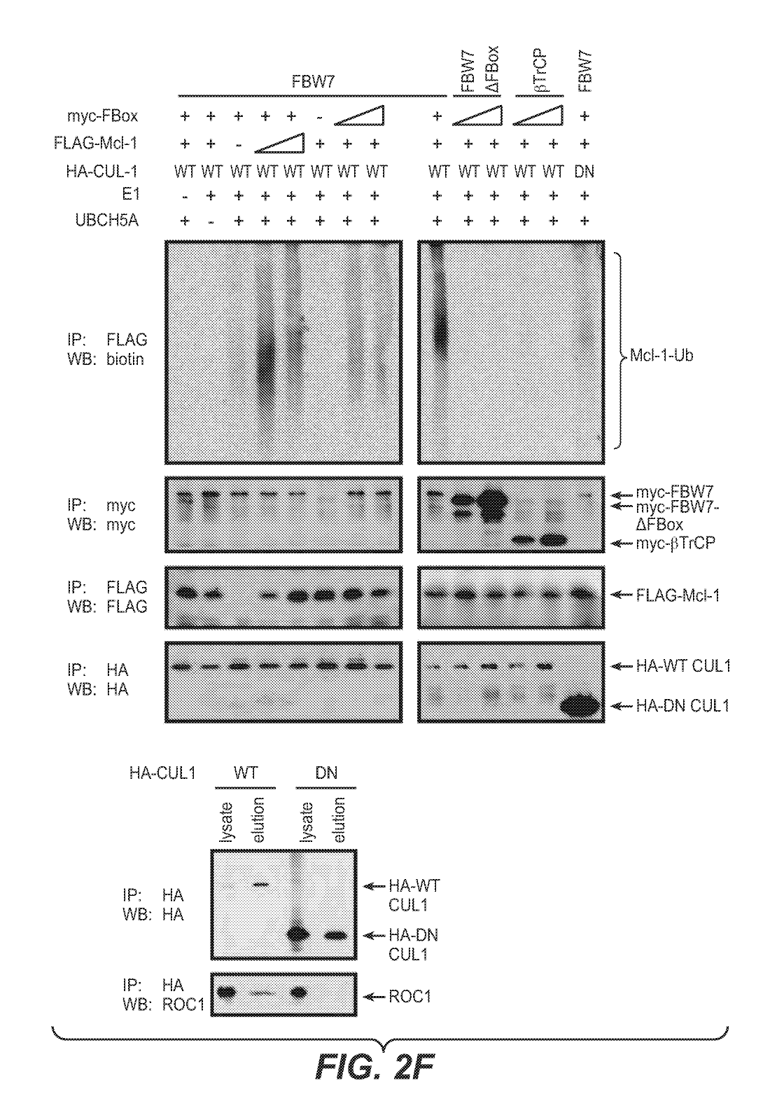

FIG. S15 shows complementation of FBW7−/− HCT116 cells with FBW7-alpha or -beta isoforms restores Mcl-1 degradation (see FIG. 2D for the accompanying figure). Expression of FLAG-FBW7 isoforms is shown.

FIGS. S16(A-E) show tandem mass spectra of Mcl-1 showing localized phosphorylation sites. FLAG-Mcl-1 purified from synchronized HCT116 cells in mitotic arrest was resolved by SDS-PAGE. Bands were excised, digested with trypsin, and analyzed by LCMS/MS on an LTQ-Orbitrap. Data were searched with Sequest (Eng et al (1994) J. Am. Soc. Mass 5(11):976-989) and phosphorylation site localization was performed using the Ascore algorithm.

S16A. Phosphorylation was localized to S64 of Mcl-1.

SEQ ID NO: 11

REIGGGEAGAVIGGSAGASPPSTLTPDSR

S16B. Phosphorylation was localized to S121 of Mcl-1 in the doubly Met-oxidized state.

SEQ ID NO: 12

AAPLEEMEAPAADAIMSPEEELDGYEPEPLGK

S16C. A peptide spanning residues R137-R176 of Mcl-1 was doubly phosphorylated. Phosphorylation at T163 could be assigned unambiguously, with the second site localized to either S159 or S162.

SEQ ID NO: 13

RPAVLPLLELVGESGNNTSTDGSLPSTPPPAEEEEDELYR

S16D. Phosphorylation of Mcl-1 residues S159 and S163 is confirmed by co-elution with an isotopically labeled synthetic peptide at a retention time of 28.54 minutes. The tandem mass spectrum of the synthetic peptide phosphorylated at residues S159 and T163 is most consistent with the second phosphate at S159.

S16E. Phosphorylation was localized to T92 of Mcl-1.

SEQ ID NO: 14

VARPPPIGAEVPDVTATPAR

FIG. S17 shows Myc-Mcl-1 is recruited to FLAG-FBW7 in mitotic arrest. The indicated constructs were expressed in HeLa cells, which were synchronized, released into paclitaxel, and processed as indicated.

FIG. S18 shows the N-terminal PEST domain of Mcl-1 is required for FBW7 binding. The indicated constructs were expressed in HeLa cells, which were synchronized, released into paclitaxel, and processed as indicated.

FIG. S19 shows evidence for cdk1, ERK, GSK3 beta, JNK, and p38 activity in mitotic arrest. HCT116 or HeLa cells were synchronized and released into paclitaxel, collected at the indicated time points, and cell lysates were blotted with the indicated antibodies. Phosphorylated cdk1, cdk1 substrates, ERK T202/Y204, and GSK3-beta Y216 are detected in mitotic arrest, as are increasing levels of JNK and p38 kinases, suggesting kinase activity. The mitotic time course follows the progression of cells in mitotic arrest: i.e. −3 is 3 hours prior to mitosis (M) and +3 is 3 hours after cells enter mitosis. A=Asynchronous cells. cdc27-P=phosphorylated cdc27.

FIGS. S20(A-B) show inhibition of GSK3 beta activity in mitotic arrest does not attenuate Mcl-1 degradation. HeLa cells were synchronized, released into paclitaxel, collected at the indicated time points. Lysates were processed and immunoblotted with the indicated antibodies.

S20A. GSK3-beta inhibitors-VIII (25 μM) or −IX (25 μM) were added when cells entered mitotic arrest.

S20B. Cells were transfected with non-specific scramble or GSK3-targeting siRNA oligos.

FIG. S21 shows pharmacologic inhibition of cdk1, JNK, and p38, but not ERK, attenuate Mcl-1 degradation in mitotic arrest. HeLa cells were synchronized, released into paclitaxel, and inhibitors of cdk1 (CGP74514A, 2 μM), ERK (FR180204, 2 μM), JNK (SP600125, 25 μM), or p38 (SB203580, 2 μM) were added when cells entered mitotic arrest. Cells were collected at the indicated time points and lysates were processed and immunoblotted with the indicated antibodies. Note: cdk1 inhibition drives cells out of mitotic arrest as indicated by the absence of cdc27 phosphorylation.

FIGS. S22(A-B) show pharmacologic inhibition of cdk, but not MEK/ERK, attenuates Mcl-1 degradation in mitotic arrest.

S22A. HeLa cells were synchronized, released into paclitaxel, and inhibitors of cdk (roscovitine, 2.5 μM) or MEK/ERK (U0126, 10 μM) were added when cells entered mitotic arrest. Cells were collected at the indicated time points and lysates were processed and immunoblotted with the indicated antibodies. Note: cdk1 inhibition drives cells out of mitotic arrest as indicated by the absence of cdc27 phosphorylation.

S22B. The efficacy and specificity of the respective inhibitors was evaluated by blotting lysates from S22A with the indicated phosphorylated substrates.

FIGS. S23(A-C) show RNAi of JNK or p38, but not ERK, attenuates Mcl-1 degradation in mitotic arrest. HeLa cells were transfected with the indicated siRNA oligos, synchronized, released into paclitaxel, and collected at the indicated time points.

S23A. Knockdown of ERK1/2 protein promoted Mcl-1 destabilization as previously reported (Domina, et al (2004) Oncogene 23:5301-5315) confounding interpretation of the kinetics of degradation in mitotic arrest. Mcl-1 band intensities were therefore quantitated in two different exposures with matched levels of Mcl-1 in the asynchronous samples (upper panels). The rate of degradation of Mcl-1 in mitotic arrest is similar with or without ERK1/2 knockdown (lower panel).

S23B. Cells were transfected with non-specific scramble or JNK-targeting siRNA oligos.

S23C. Cells were transfected with non-specific scramble or p38-targeting siRNA oligos.

FIGS. S24(A-C) show inhibition of cdk1 or CKII attenuates Mcl-1 degradation in mitotic arrest. HeLa cells were transfected as indicated, synchronized, released into paclitaxel, collected at the indicated time points, and lysates were processed and immunoblotted with the indicated antibodies.

S24A. A myc-tagged version of non-degradable cyclin B1 (myc-Δcyclin B1) was transfected to maintain cells in mitotic arrest upon cdk1 inhibition Inhibitors of cdk1 (CGP74514A, 2 μM or roscovitine, 2.5 μM) were added when cells entered mitotic arrest.

S24B. Expression of cdc20 was knocked down with RNAi oligos to maintain cells in mitotic arrest upon cdk1 inhibition. Inhibitors of cdk1 (CGP74514A, 2 μM or roscovitine, 2.5 μM) were added when cells entered mitotic arrest. Asterisks indicate cdc20 below a background band.

S24C. Cells were transfected with non-specific scramble or CKII-targeting siRNA oligos. A CKII band shift is evident when cells enter mitotic arrest, suggesting kinase activity.

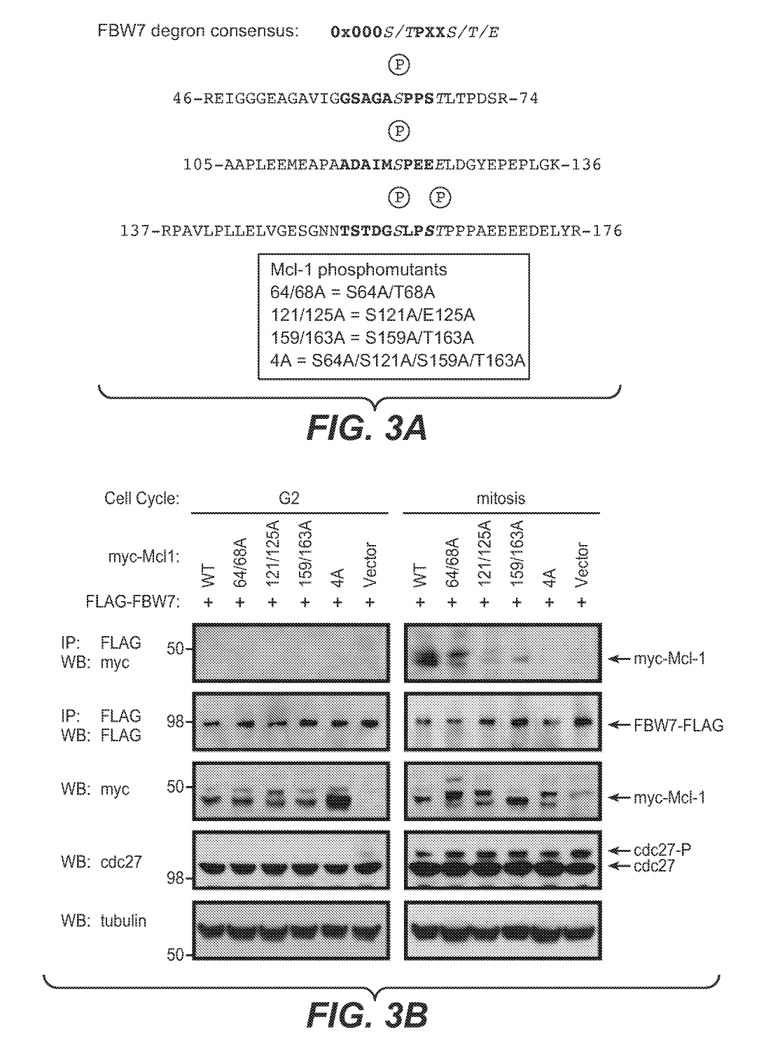

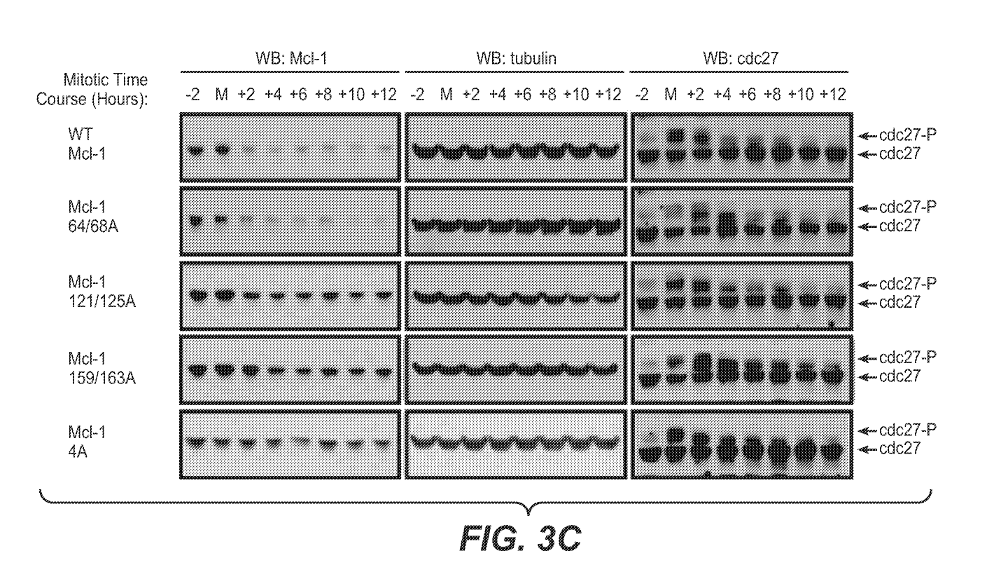

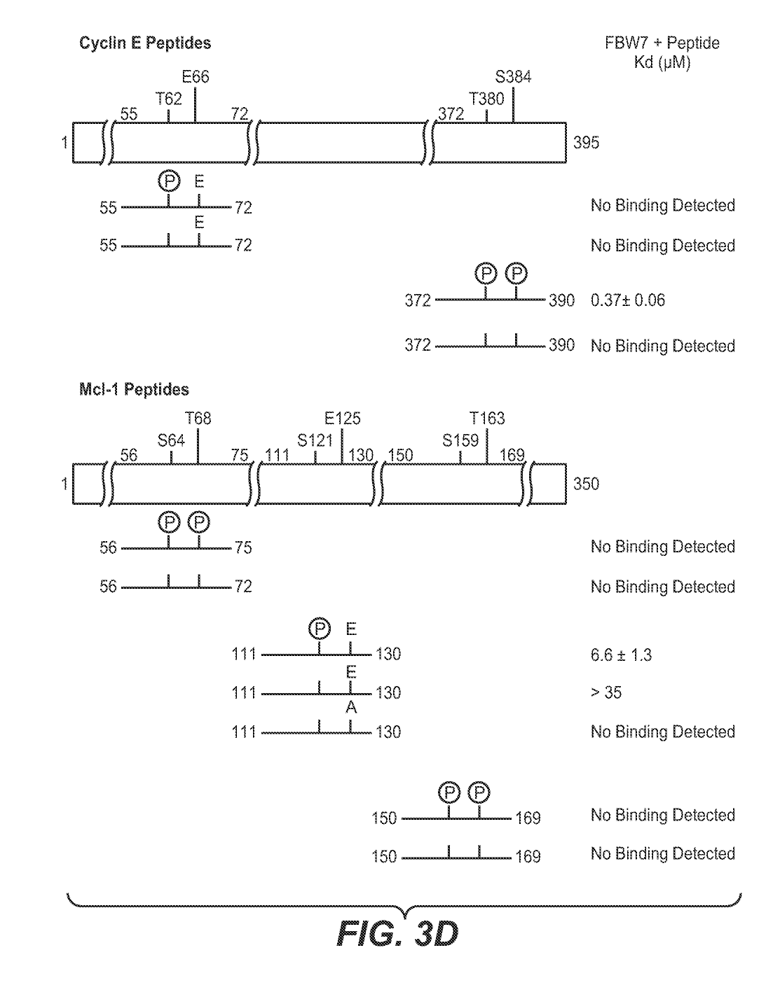

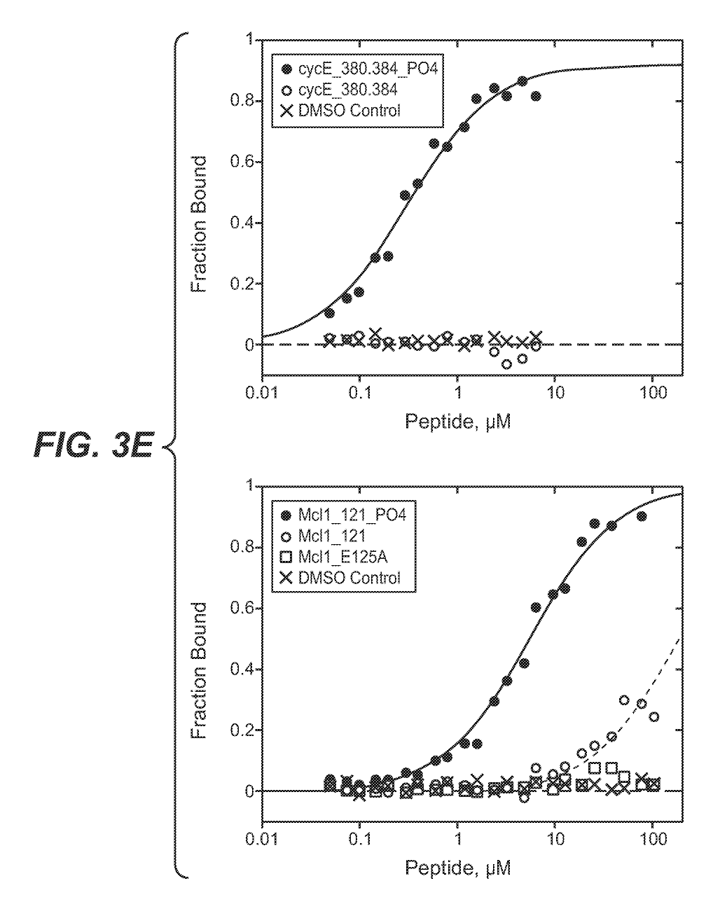

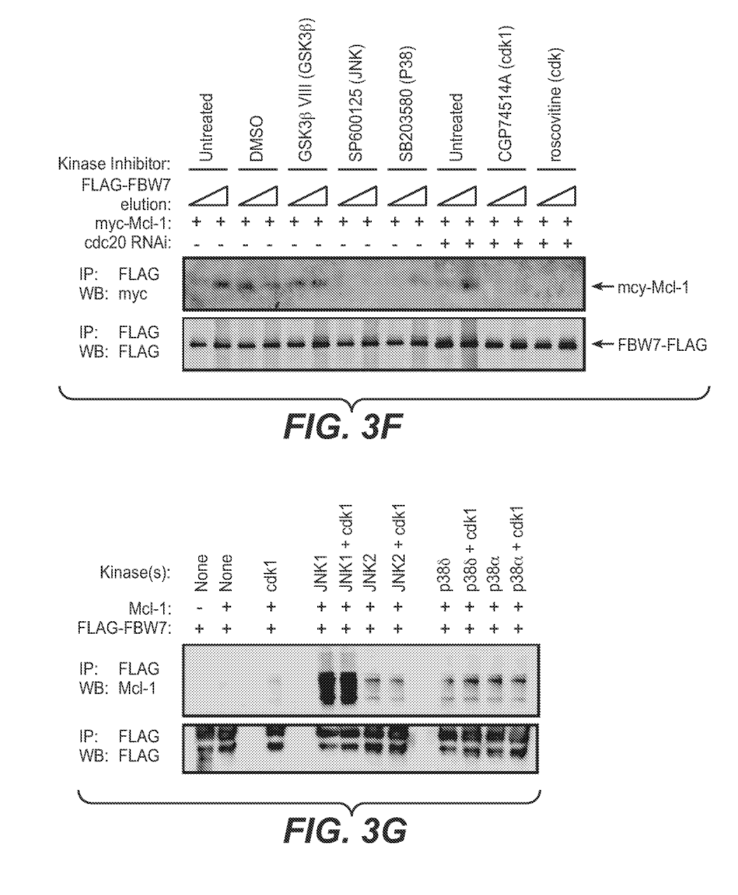

FIG. S25 shows Western blot analysis of lysates from FIG. 3F. Pharmacologic inhibition of JNK, p38, or cdk1 attenuates recruitment of myc-Mcl-1 to FLAG-FBW7 in mitotic arrest. The indicated constructs were expressed in HeLa cells with or without scramble or cdc20 RNAi, and then synchronized and released into paclitaxel. When cells entered mitotic arrest the indicated agents were added for 1 hour followed by a 3 hour incubation with 25 μM MG-132 prior to collection: 0.1% DMSO, GSK3 beta (GSK3 beta inhibitor-VIII, 25 μM), JNK (SP600125, 25 μM), p38 (SB203580, 2.65 μM), cdk1 (CGP74514A, 404), or cdk (roscovitine, 2.5 μM). Cells were subsequently collected and processed as indicated.

FIG. S26 shows RNAi of JNK attenuates recruitment of myc-Mcl-1 to FLAG-FBW7 in mitotic arrest. The indicated constructs were expressed in HeLa cells with or without scramble or JNK RNAi, synchronized, and released into paclitaxel. Cells were incubated with 25 μM MG-132 for 3 hours upon entry into mitotic arrest, collected, and processed as indicated.

FIGS. S27(A-C) show T92 regulates Mcl-1 turnover in mitotic arrest via PP2A binding.

S27A. The T92A Mcl-1 phosphomutant is protected from degradation in mitotic arrest. The Hela cells were transfected with the indicated constructs, synchronized, released into paclitaxel, and collected at the indicated time points.

S27B. Association of endogenous PP2A with FLAG-Mcl-1 phosphomutant T92A is stabilized in mitotic arrest. The indicated constructs were expressed in HeLa cells that were synchronized, released into paclitaxel, and processed as indicated. Normalized amounts of FLAG-Mcl-1 elutions were used to best compare levels of associated endogenous PP2A

S27C. Decreased associated endogenous PP2A protein and PP2A activity with Mcl-1 in mitotic arrest. HeLa cells were synchronized, released into paclitaxel, and processed as indicated. Mcl-1 immunoprecipitates from mitotic and post-mitotic cells were evaluated as these samples had the most comparable levels of endogenous Mcl-1, thus permitting the most accurate assessment of associated PP2A protein and activity.

FIGS. S28(A-B) show washing out anti-tubulin chemotherapeutics from cells in mitotic arrest decreases JINX, p38, and cdk1 kinase activity and stabilizes Mcl-1. HeLa or HCT116 cells were synchronized and released into nocodazole or paclitaxel in duplicate. When cells entered mitotic arrest nocodazole or paclitaxel was washed out of half of the samples as noted. Cells were collected and processed as indicated.

FIG. S29 shows Bak and Bax are activated in mitotic arrest. HeLa or HCT116 cells were synchronized and released into paclitaxel in duplicate. Cells were collected at the indicated time points and collected in buffers with the indicated detergent: CHAPS maintains Bak and Bax in the native state while Triton-X100 induces the active Bak and Bax conformations and is thus a positive control. Lysates were immunoprecipitated with conformation-specific Bak or Bax antibodies and immunoprecipitates or whole cell lysates were probed with antibodies recognizing total Bak or Bax or the indicated proteins.

FIG. S30 shows recruitment of myc-Mcl-1 to FLAG-FBW7 in mitotic arrest is compromised by FBW7 mutations. The indicated constructs were expressed in HeLa cells, which were synchronized and released into paclitaxel and processed as indicated. The FBW7 mutations from the corresponding patient-derived cell lines are listed below.

FIGS. S31(A-B) show FBW7−/− colon cancer cell lines are more resistant to paclitaxel-induced cell death and show attenuated Mcl-1 degradation in mitotic arrest relative to FBW7− WT parental cell lines. Unsynchronized cell lines (with FBW7 status specified in parentheses) were treated with various concentrations of paclitaxel or vincristine for 48 hours prior to cell viability assessment. Synchronized cells were released into paclitaxel or vincristine and were collected at the indicated time points for western blot analysis.

FIG. S32 shows analysis of Mcl-1 message in mitotic arrest. DLD1, HCT116 or HeLa cells were synchronized, released into 200 nM vincristine, and collected at the indicated time points.

FIGS. S33(A-B) show FBW7−/− or FBW7 mutant colon cancer cell lines are more resistant to paclitaxel-induced cell death and show attenuated Mcl-1 degradation in mitotic arrest relative to FBW7-WT cell lines. The unsynchronized, indicated cell lines (with FBW7 status specified in parentheses) were treated with various concentrations of paclitaxel for 48 hours prior to cell viability assessment. Synchronized cells were released into paclitaxel and collected at the indicated time points for western blot analysis.

FIG. S34 shows asynchronous ovarian cancer cell lines are arrested in mitosis by exposure to paclitaxel. The unsynchronized cell lines (with FBW7 status specified in parentheses) were treated with 200 nM paclitaxel and were subsequently collected at the indicated time points for western blot and phospho-histone H3 ELISA analysis. The TOV21G cell line is only transiently arrested in mitosis as indicated by phospho-cdc27 immunoblotting and phospho-histone H3 ELISA analysis, and has attenuated Mcl-1 degradation comparable to the FBW7 mutant cell line SKOV3.

FIGS. S35(A-D) show FBW7 inactivation promotes anti-tubulin agent resistance in ovarian tumor xenografts in vivo.

S35A. FBW7-mutant ovarian tumors are more resistant to paclitaxel-induced cell death in vivo relative to FBW7-WT ovarian tumors. Growth curves for TOV112D-X1 ovarian tumors with wild-type FBW7 expressing an empty vector (vector; n=8; blue line) or mutant FBW7 (FBW7-R505L; n=12; red line) grown as xenografts under the kidney capsule of athymic nu/nu mice. paclitaxel was administered on days 21 and 23 post implant (green arrows). Data are presented as the mean±SEM of the tumor volumes. *P=0.0004. **P=0.02.

S35B. Western blot analysis of tumor lysates from the indicated xenograft tumors harvested on day 26 post-implant.

S35C. A graphical summary of Mcl-1 expression in xenograft lysates normalized to GAPdH levels in the corresponding tumors.

S35D. A graphical summary of Bcl-XL expression in xenograft lysates normalized to GAPdH levels in the corresponding tumors.

FIG. S36 shows sensitivity to paclitaxel-induced cell death is restored in FBW7−/− cells upon Mcl-1 ablation. Wild-type (WT) or FBW7−/− HCT116 cells were transduced with the indicated doxycycline-inducible shRNA constructs, cultured in the presence of 0.2 μg/mL doxycycline, and treated with various concentrations of paclitaxel for 48 hours prior to cell viability assessment. Data are presented as the mean±SEM, n=3. Immunoblots of cell extracts are also shown.

FIG. S37 shows Mcl-1 expression modulates mitotic slippage in FBW7-deficient cells following exposure to vincristine. Wild-type or FBW7−/− HCT116 cells were transduced with the indicated doxycycline-inducible shRNA constructs, cultured in the presence of doxycycline, treated with 200 nM Vincristine, and harvested at designated time points for western blot analysis with the indicated antibodies. A=asynchronous cells.

FIG. S38 shows examples of time-lapse sequences depicting the indicated fates of HCT116 cells treated with paclitaxel or vincristine. Division is illustrated by formation of a metaphase plate and subsequent chromosomal segregation. Apoptosis is indicated by the characteristic condensation of chromatin and formation of apoptotic bodies. Mitotic slippage is indicated by mitotic exit in the absence of anaphase initiation. Scale bar=10 μM.

FIG. S39 shows genetic interaction between FBW7 and MCL1 in human ovarian cancers. Red dotted lines represent cutoffs for copy number gains (log 2ratio≧0.3), and blue dotted lines indicate cutoffs for copy number losses (log 2ratio≦−0.3). Among the 318 primary tumor samples pooled from six datasets, 94 harbor FBW7 deletion and 86 have MCL-1 amplification. MCL-1 copy number gain, FBW7 copy number loss, or both alterations were detected in 44% of the tumors and both genetic events occur coincidentally in 40 samples (green points), significantly more frequently than random (odds ratio=2.86, p-value=6.8e-5, one-tailed Fisher's exact test), suggesting association.

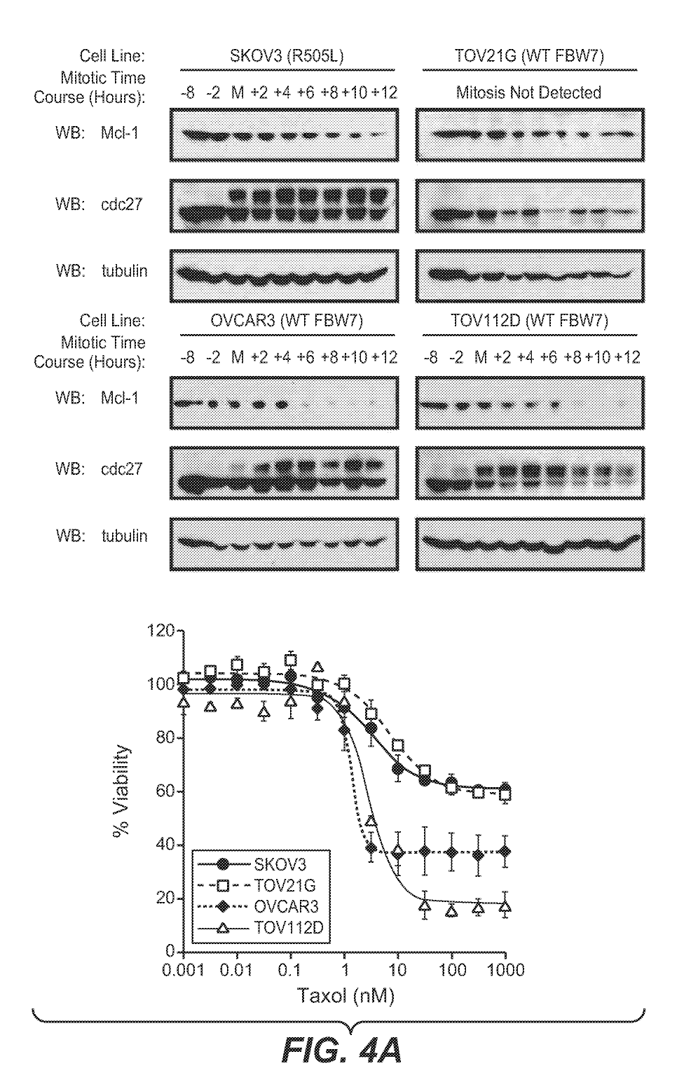

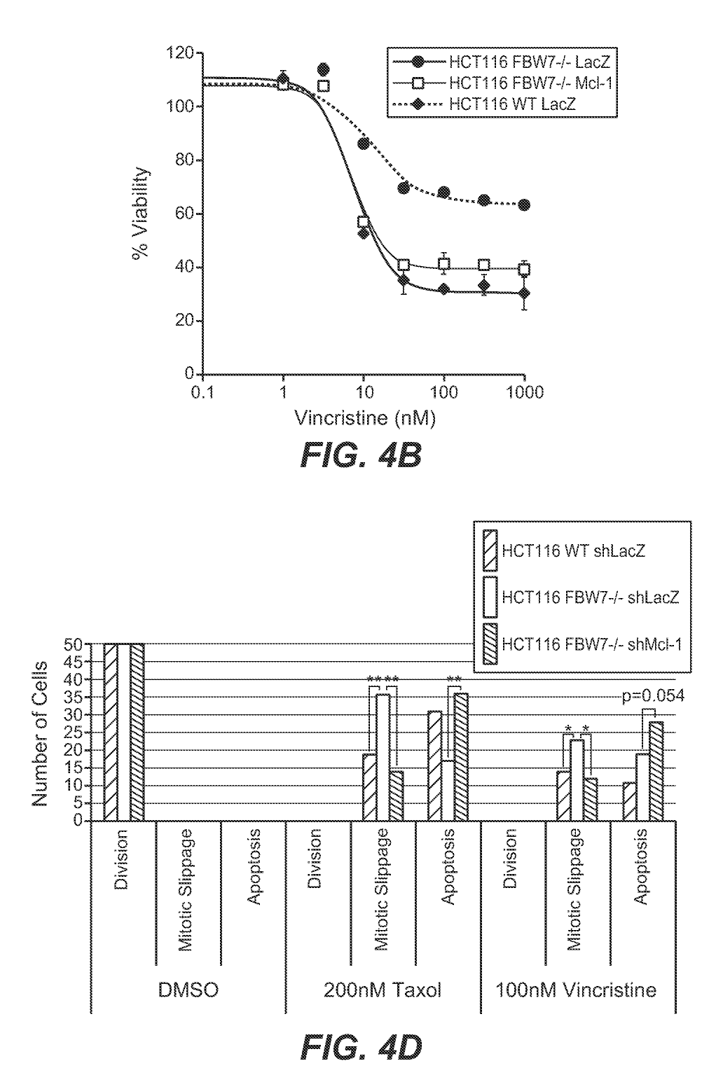

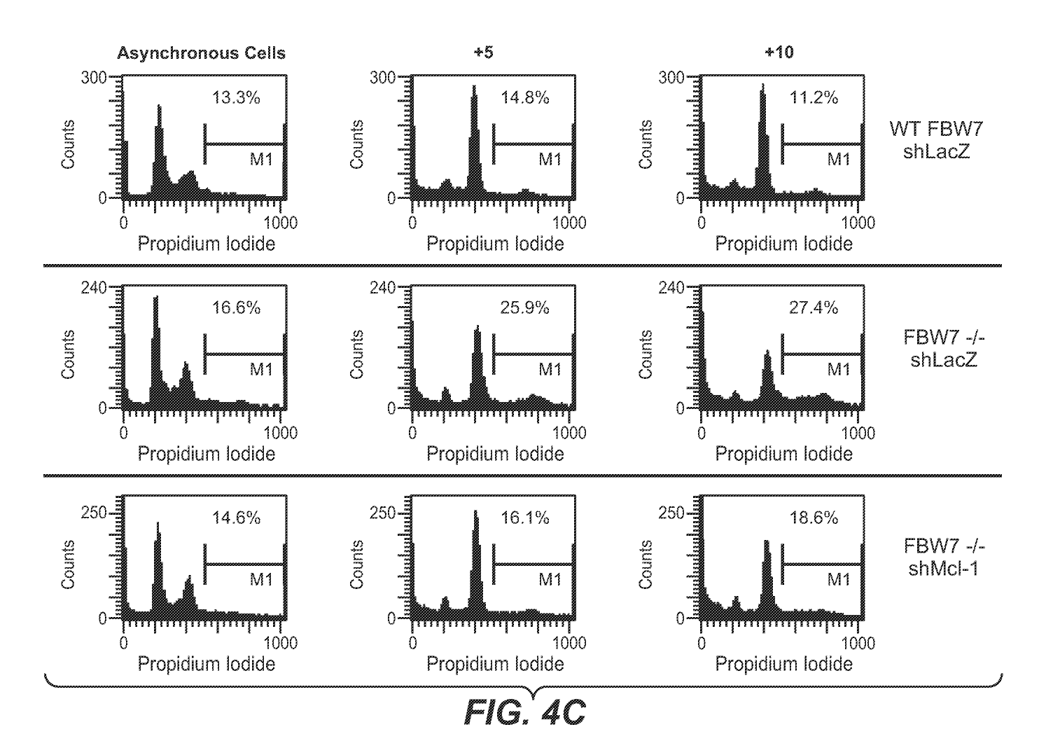

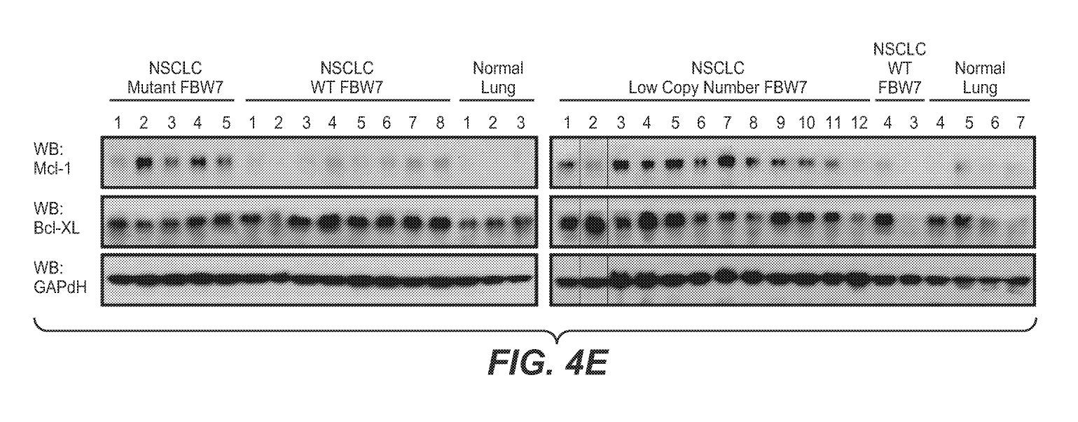

FIG. Supplemental Tables 2A,B show patient sample mutation and copy number alteration status. SRCID=patient designator ID. Gel sample #: corresponds to the gels in FIG. 4E. Tissue, Mutation (Nucleic acid), Mutation (Amino acid) refer to FBW7 mutations. Mutation status: MUT=mutant FBW7, WT=wild-type FBW7. * Mutations are reported with reference to FBW7-beta isoform, Genbank sequence NM_018315.3. † Limits were set at <1.6 copies for loss and >2.5 copies for gain. NSCLC=Non-Small Cell Lung Cancer. References: 1—Peters, B. A. et al. (2007) “Highly efficient somatic-mutation identification using Escherichia coli mismatch-repair detection.” Nat. Methods 4, 713-715. 2-Kan, Z. et al. (2010) Diverse somatic mutation patterns and pathway alterations in human cancers.” Nature 466(7308):869-873. ND=not determined. N/A=not applicable

### DEFINITIONS

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., a lung cancer. “Diagnosis” may also refer to the classification of a particular type of cancer, e.g., by histology (e.g., a non small cell lung carcinoma), by molecular features (e.g., a lung cancer characterized by nucleotide and/or amino acid variation(s) in a particular gene or protein), or both.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity 5 of treatment.

“Patient response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

“Change” or “modulation” of the status of a biomarker, including Mcl-1 and FBW7, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry, immunocytochemistry, ELISA, or mass spectrometry whereby degradation, stabilization, or post-translational modifications of the proteins such as phosphorylation or ubiquitination is detected.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and mesothelioma. Gastric cancer, as used herein, includes stomach cancer, which can develop in any part of the stomach and may spread throughout the stomach and to other organs; particularly the esophagus, lungs, lymph nodes, and the liver.

The term “hematopoietic malignancy” refers to a cancer or hyperproliferative disorder generated during hematopoiesis involving cells such as leukocytes, lymphocytes, natural killer cells, plasma cells, and myeloid cells such as neutrophils and monocytes. Hematopoietic malignancies include non-Hodgkin's lymphoma, diffuse large hematopoietic lymphoma, follicular lymphoma, mantle cell lymphoma, chronic lymphocytic leukemia, multiple myeloma, acute myelogenous leukemia, and myeloid cell leukemia. Lymphocytic leukemia (or “lymphoblastic”) includes Acute lymphoblastic leukemia (ALL) and Chronic lymphocytic leukemia (CLL). Myelogenous leukemia (also “myeloid” or “nonlymphocytic”) includes Acute myelogenous (or Myeloblastic) leukemia (AML) and Chronic myelogenous leukemia (CML).

Hematopoietic malignancies also include the diseases listed in Table 1, the WHO classification of Human Hematopoietic Malignancies; Tumors of Hematopoietic and Lymphoid Tissues (Jaffe E. S., Harris N. L., Stein H., Vardiman J. W. (Eds.) (2001): World Health Organization Classification of Tumours. Pathology and Genetics of Tumours of Hematopoietic and Lymphoid Tissues. IARC Press: Lyon) with the morphology code of the International Classification of Diseases (ICD-O). Behavior is coded/3 for malignant tumors and /1 for lesions of low or uncertain malignant potential.

TABLE 1

| | |
|---|---|
| I. | CHRONIC MYELOPROLIFERATIVE DISEASES |
| | Chronic myelogenous leukemia-ICD-O 9875/3 |
| | Chronic neutrophilic leukemia-ICD-O 9963/3 |
| | Chronic eosinophilic leukemia/hypereosinophilic syndrome-ICD-O 9964/3 |
| | Polycythemia vera-ICD-O 9950/3 |
| | Chronic idiopathic myelofibrosis-ICD-O 9961/3 |
| | Essential thrombocytemia-ICD-O 9962/3 |
| | Chronic Myeloproliferative disease, unclassifiable-ICD-O 9975/3 |
| II. | MYELODYSPLASTIC/MYELOPROLIFERATIVE DISEASES |
| | Chronic myelomonocytic leukemia-ICD-O 9980/3 |
| | Atypical chronic myelogenous leukemia-ICD-O 9876/3 |
| | Juvenile myelomonocytic leukemia-ICD-O 9946/3 |
| | Myelodysplastic/myeloproliferative diseases, unclassifiable-ICD-O 9975/3 |
| III. | MYELODYSPLASTIC SYNDROMES |
| | Refractory anemia-ICD-O 9980/3 |
| | Refractory anemia with ringed sideroblasts-ICD-O 9982/3 |
| | Refractory cytopenia with multilineage dysplasia-ICD-O 9985/3 |
| | Refractory anemia with excess blasts-ICD-O 9983/3 |
| | Myelodysplastic syndrome associated with isolated del(5q) chromosome |
| | abnormality-ICD-O 9986/3 |
| | Myelodysplastic syndrome, unclassifiable 9989/3 |
| IV. | ACUTE MYELOID LEUKEMIAS (AML) |
| | Acute myeloid leukemias with recurrent cytogenetic abnormalities |
| | AML with t(8;21)(q22;q22), AML1/ETO-ICD-O 9896/3 |
| | AML with inv(16)(p13q22) or t(16;16)(p13;q22), CBFb/MYH11-ICD-O 9871/3 |
| | Acute promyelocytic leukemia (AML with t(15;17)(q22;q12), |
| | PML-RARa and variants)-ICD-O 9866/3 |
| | AML with 11q23 (MLL) abnormalities-ICD-O 9897/3 |
| | Acute myeloid leukemia multilineage dysplasia-ICD-O 9895/3 |
| | Acute myeloid leukemia and myelodysplastic syndrome, |
| | therapy related-ICD-O 9920/3 |
| | Acute myeloid leukemia not otherwise categorized |
| | Acute myeloid leukemia, minimally differentiated-ICD-O 9872/3 |
| | Acute myeloid leukemia, without maturation-ICD-O 9873/3 |
| | Acute myeloid leukemia, with maturation-ICD-O 9874/3 |
| | Acute myelomonocytic leukemia-ICD-O 9867/3 |
| | Acute monoblastic and monocytic leukemia-ICD-O 9891/3 |
| | Acute erythroid leukemia-ICD-O 9840/3 |
| | Acute megakaryoblastic leukemia-ICD-O 9910/3 |
| | Acute basophilic leukemia-ICD-O 9870/3 |
| | Acute panmyelosis with myelofibrosis-ICD-O 9931/3 |
| | Myeloid sarcoma-ICD-O 9930/3 |
| | Acute leukemia of ambiguous lineage-ICD-O 9805/3 |
| V. | B-CELL NEOPLASMS |
| | Precursor hematopoietic neoplasm |
| | Precursor B lymphoblastic leukemia/-ICD-O 9835/3 |
| | lymphoma-ICD-O 9728/3 |
| | Mature hematopoietic neoplasm |
| | Chronic lymphocytic leukemia (CLL)-ICD-O 9823/3 |
| | small lymphocytic lymphoma-ICD-O 9670/3 |
| | hematopoietic prolymphocytic leukemia-ICD-O 9833/3 |
| | Lymphoplasmacytic lymphoma-ICD-O 9671/3 |
| | Splenic marginal zone lymphoma-ICD-O 9689/3 |
| | Hairy cell leukemia-ICD-O 9940/3 |
| | Plasma cell myeloma-ICD-O 9732/3 |
| | Solitary plasmacytoma of bone-ICD-O 9731/3 |
| | Extraosseous plasmacytoma-ICD-O 9734/3 |
| | Extranodal marginal zone hematopoietic lymphoma of mucosa-associated lymphoid |
| | tissue (MALT-lymphoma)-ICD-O 9699/3 |
| | Nodal marginal zone hematopoietic lymphoma-ICD-O 9699/3 |
| | Follicular lymphoma-ICD-O 9690/3 |
| | Mantle cell lymphoma)-ICD-O 9673/3 |
| | Diffuse large hematopoietic lymphoma-ICD-O 9680/3 |
| | Mediastinal (thymic) large cell lymphoma-ICD-O 9679/3 |
| | Intravascular large hematopoietic lymphoma-ICD-O 9680/3 |
| | Primary effusion lymphoma-ICD-O 9678/3 |
| | Burkitt lymphoma/-ICD-O 9687/3 |
| | leukemia-ICD-O 9826/3 |
| | hematopoietic proliferations of uncertain malignant potential |
| | Lymphomatoid granulomatosis-ICD-O 9766/1 |
| | Post-transplant lymphoproliferative disorder, pleomorphic-ICD-O 9970/1 |
| VI. | T-CELL AND NK-CELL NEOPLASMS |
| | Precursor T-cell neoplasms |
| | Precursor T lymphoblastic leukemia/-ICD-O 9837/3 |
| | lymphoma-ICD-O 9729/3 |
| | Blastic NK cell lymphoma-ICD-O 9727/3 |
| | Mature T-cell and NK-cell neoplasms |
| | T-cell prolymphocytic leukemia-ICD-O 9834/3 |
| | T-cell large granular lymphocytic leukemia-ICD-O 9831/3 |
| | Aggressive NK cell leukemia-ICD-O 9948/3 |
| | Adult T-cell leukemia/lymphoma-ICD-O 9827/3 |
| | Extranodal NK/T cell lymphoma, nasal type-ICD-O 9719/3 |
| | Enteropathy type T-cell lymphoma-ICD-O 9717/3 |
| | Hepatosplenic T-cell lymphoma-ICD-O 9716/3 |
| | Subcutaneous panniculitis-like T-cell lymphoma-ICD-O 9708/3 |
| | Mycosis fungoides-ICD-O 9700/3 |
| | Sezary Syndrome-ICD-O 9701/3 |
| | Primary cutaneous anaplastic large cell lymphoma-ICD-O 9718/3 |
| | Peripheral T-cell lymphoma, unspecified-ICD-O 9702/3 |
| | Angioimmunoblastic T-cell lymphoma-ICD-O 9705/3 |
| | Anaplastic large cell lymphoma-ICD-O 9714/3 |
| | T-cell proliferation of uncertain malignant potential |
| | Lymphomatoid papulosis-ICD-O 9718/1 |
| VII | HODGKIN LYMPHOMA |
| | Nodular lymphocyte predominant Hodgkin lymphoma-ICD-O 9659/3 |
| | Classical Hodgkin lymphoma-ICD-O 9650/3 |
| | Nodular sclerosis classical Hodgkin lymphoma-ICD-O 9663/3 |
| | Lymphocyte-rich classical Hodgkin lymphoma-ICD-O 9651/3 |
| | Mixed cellularity classical Hodgkin lymphoma-ICD-O 9652/3 |
| | Lymphocyte-depleted classical Hodgkin lymphoma-ICD-O 9653/3 |
| VIII. | HISTIOCYTIC AND DENDRITIC-CELL NEOPLASMS |
| | Macrophage/histiocytic neoplasm |
| | Histiocytic sarcoma-ICD-O 9755/3 |
| | Dendritic cell neoplasms |
| | Langerhans cell histiocytosis-ICD-O 9751/1 |
| | Langerhans cell sarcoma-ICD-O 9756/3 |
| | Interdigitating dendritic cell sarcoma/tumor-ICD-O 9757/3/1 |
| | Follicular dendritic cell sarcoma/tumor-ICD-O 9758/3/1 |
| | Dendritic cell sarcoma, not otherwise specified-ICD-O 9757/3 |
| IX. | MASTOCYTOSIS |
| | Cutaneous mastocytosis |
| | Indolent systemic mastocytosis-ICD-O 9741/1 |
| | Systemic mastocytosis with associated clonal, hematological non-mast cell |
| | lineage disease-ICD-O 9741/3 |
| | Aggressive systemic mastocytosis-ICD-O 9741/3 |
| | Mast cell leukemia-ICD-O 9742/3 |
| | Mast cell sarcoma-ICD-O 9740/3 |
| | Extracutaneous mastocytoma-ICD-O 9740/1 |

The term “hyperproliferative disorder” refers to a condition manifesting some degree of abnormal cell proliferation. In one embodiment, a hyperproliferative disorder is cancer.

“Tumor” refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “chemotherapeutic agent” is a biological (large molecule) or chemical (small molecule) compound useful in the treatment of cancer, regardless of mechanism of action.

An “anti-tubulin chemotherapeutic agent” is a chemotherapeutic compound that has properties related to disruption, modulation, stabilization, or inhibition of the normal function of the tubulin family of globular proteins that make up microtubules and are associated with mitosis. Examples of anti-tubulin chemotherapeutic agents include, but are not limited to, paclitaxel (TAXOL®), docetaxel (TAXOTERE®), vincristine, vinblastine, vinorelbine (NAVELBINE®), eribulin (HALAVEN®), combretastatin, maytansines, dolastatins, auristatins, and the antibody-drug conjugates thereof. Anti-tubulin chemotherapeutic agents include mitotic kinase inhibitor compounds that promote mitotic arrest, such as PLK, Aurora, and KSP inhibitors (Inuzuka et al (2011) Nature. 2011 Mar. 3; 471(7336):104-9.

The term “mammal” includes, but is not limited to, humans, mice, rats, guinea pigs, monkeys, dogs, cats, horses, cows, pigs, and sheep.

The term “antibody” herein is used in the broadest sense and specifically covers monoclonal antibodies, polyclonal antibodies, multispecific antibodies (e.g. bispecific antibodies) formed from at least two intact antibodies, and antibody fragments, so long as they exhibit the desired biological activity.

“ELISA” (Enzyme-linked immunosorbent assay) is a popular format of a “wet-lab” type analytic biochemistry assay that uses one sub-type of heterogeneous, solid-phase enzyme immunoassay (EIA) to detect the presence of a substance in a liquid sample or wet sample (Engvall E, Perlman P (1971). “Enzyme-linked immunosorbent assay (ELISA). Quantitative assay of immunoglobulin G”. Immunochemistry 8 (9): 871-4; Van Weemen B K, Schuurs A H (1971). “Immunoassay using antigen-enzyme conjugates”. FEBS Letters 15 (3): 232-236). ELISA can perform other forms of ligand binding assays instead of strictly “immuno” assays, though the name carried the original “immuno” because of the common use and history of development of this method. The technique essentially requires any ligating reagent that can be immobilized on the solid phase along with a detection reagent that will bind specifically and use an enzyme to generate a signal that can be properly quantified. In between the washes only the ligand and its specific binding counterparts remain specifically bound or “immunosorbed” by antigen-antibody interactions to the solid phase, while the nonspecific or unbound components are washed away. Unlike other spectrophotometric wet lab assay formats where the same reaction well (e.g. a cuvette) can be reused after washing, the ELISA plates have the reaction products immunosorbed on the solid phase which is part of the plate and thus are not easily reusable. Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are not specifically bound. After the final wash step, the plate is developed by adding an enzymatic substrate to produce a visible signal, which indicates the quantity of antigen in the sample.

“Immunohistochemistry” (IHC) refers to the process of detecting antigens (e.g., proteins) in cells of a tissue section by exploiting the principle of antibodies binding specifically to antigens in biological tissues. Immunohistochemical staining is widely used in the diagnosis of abnormal cells such as those found in cancerous tumors. Specific molecular markers are characteristic of particular cellular events such as proliferation or cell death (apoptosis). IHC is also widely used to understand the distribution and localization of biomarkers and differentially expressed proteins in different parts of a biological tissue. Visualising an antibody-antigen interaction can be accomplished in a number of ways. In the most common instance, an antibody is conjugated to an enzyme, such as peroxidase, that can catalyse a colour-producing reaction (see immunoperoxidase staining) Alternatively, the antibody can also be tagged to a fluorophore, such as fluorescein or rhodamine (see immunofluorescence).

“Immunocytochemistry” (ICC) is a common laboratory technique that uses antibodies that target specific peptides or protein antigens in the cell via specific epitopes. These bound antibodies can then be detected using several different methods. ICC can evaluate whether or not cells in a particular sample express the antigen in question. In cases where an immunopositive signal is found, ICC also determines which sub-cellular compartments are expressing the antigen.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton Pa.; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the mammal being treated therewith.

Induced myeloid leukemia cell differentiation protein “Mcl-1” is also referred to as BCL2L3; EAT; MCL1-ES; MCL1L; MCL1S; MGC104264; MGC1839; Mcl-1; TM; bcl2-L-3; or mcl1/EAT, and is encoded by the MCL1 gene (Kozopas et al (1993) Proc Natl Acad Sci USA. 90(8):3516-3520; Craig et al (1995) Genomics 23(2):457-463; Harley et al (2010) EMBO J. July 21; 29(14):2407-20. Epub 2010 Jun. 4).

A “degron” is a specific sequence of amino acids in a protein that directs protein substrate degradation. A degron sequence can occur at either the N or C-terminal region, these are called N-Degrons or C-degrons respectively. A temperature sensitive degron takes advantage of the N-end rule pathway, in which a destabilizing N-terminal residue dramatically decreases the in vivo half-life of a protein (Dohmen et al (1994) Science 263(5151):1273-1276). In this example, the degron is a fusion protein of ubiquitin, arginine, and DHFR. DHFR is dihydrofolate reductase, a mouse-derived enzyme that functions in the synthesis of thymine. It is also heat-labile—at a higher temperature of 37° C., becomes slightly unfolded and exposes an internal lysine, the site of poly-ubiquitination. Internal residues can also comprise degrons. Degron residues may be post-translationally modified, for example by phosphorylation or hydroxylation, to direct binding to ubiquitin ligases. Ubiquitin ligase association promotes ubiquitination and subsequent proteasomal degradation. Proteolysis is highly processive, and the protein is degraded by the proteasome. The degron can be fused to a gene to produce the corresponding temperature-sensitive protein. It is portable, and can be transferred on a plasmid.

“FBW7”, also known as FBXW7, is a haplo-in-sufficient tumor suppressor that targets proto-oncoproteins for degradation including c-myc, c-jun, NOTCH, and cyclin E (FBW7 beta isoform: Genbank sequence NM_018315.3)(Welcker, M. and Clurman, B. E. (2008) Nature reviews 8:83-93). F-box/WD repeat-containing protein 7 is a protein that in humans is encoded by the FBXW7 gene (Winston J T, et al (1999). Curr Biol 9 (20): 1180-2; Gupta-Rossi N, et al (2001) J Biol Chem 276 (37): 34371-8; WO 2010/030865). The FBXW7 gene encodes a member of the F-box protein family which is characterized by an approximately 40 amino acid motif, the F-box. The F-box proteins constitute one of the four subunits of ubiquitin protein ligase complex called SCFs (SKP1-cullin-F-box), which function in phosphorylation-dependent ubiquitination. The F-box proteins are divided into 3 classes: Fbws containing WD-40 domains, Fbls containing leucine-rich repeats, and Fbxs containing either different protein-protein interaction modules or no recognizable motifs. The protein encoded by this gene was previously referred to as FBX30, and belongs to the Fbws class; in addition to an F-box, this protein contains 7 tandem WD40 repeats. This protein binds directly to cyclin E and probably targets cyclin E for ubiquitin-mediated degradation. Mutations in this gene are detected in ovarian and breast cancer cell lines, implicating the gene's potential role in the pathogenesis of human cancers. Three transcript variants encoding three different isoforms have been found for this gene. FBW7 is an F-box/WD repeat-containing protein that in humans is encoded by the FBXW7 gene. This gene encodes a member of the F-box protein family which is characterized by an approximately 40 amino acid motif, the F-box. The F-box proteins constitute one of the four subunits of ubiquitin protein ligase complex called SCFs (SKP1-cullin-F-box), which function in phosphorylation-dependent ubiquitination. The F-box proteins are divided into 3 classes: Fbws containing WD-40 domains, Fbls containing leucine-rich repeats, and Fbxs containing either different protein-protein interaction modules or no recognizable motifs. The protein encoded by this gene was previously referred to as FBX30, and belongs to the Fbws class; in addition to an F-box, this protein contains 7 tandem WD40 repeats. This protein binds directly to cyclin E and probably targets cyclin E for ubiquitin-mediated degradation. Mutations in this gene are detected in ovarian and breast cancer cell lines, implicating the gene's potential role in the pathogenesis of human cancers. Transcript variants encoding three different isoforms have been found for this gene.

### DETAILED DESCRIPTION OF EXEMPLARY EMBODIMENTS

Pro-survival protein Mcl-1 is a critical regulator of apoptosis triggered by anti-tubulin chemotherapeutics. During mitotic arrest, Mcl-1 declines dramatically via a post-translational mechanism to potentiate cell death. Phosphorylation of Mcl-1 directs its interaction with the FBW7 tumor suppressor, the substrate-binding component of a ubiquitin ligase complex. Polyubiquitination of Mcl-1 then targets it for proteasomal degradation. FBW7 deletion or loss of function mutations identified in patient-derived tumor samples blocked Mcl-1 degradation, conferred resistance to antimitotic agents, and promoted chemotherapeutic-induced polyploidy. Primary tumor samples were enriched for FBW7 both inactivation and Mcl-1 elevation, underscoring their prominent roles in oncogenesis. Profiling the FBW7 and Mcl-1 status of tumors could identify patients that will, or will not, obtain the full pro-apoptotic benefit of anti-tubulin chemotherapeutics.

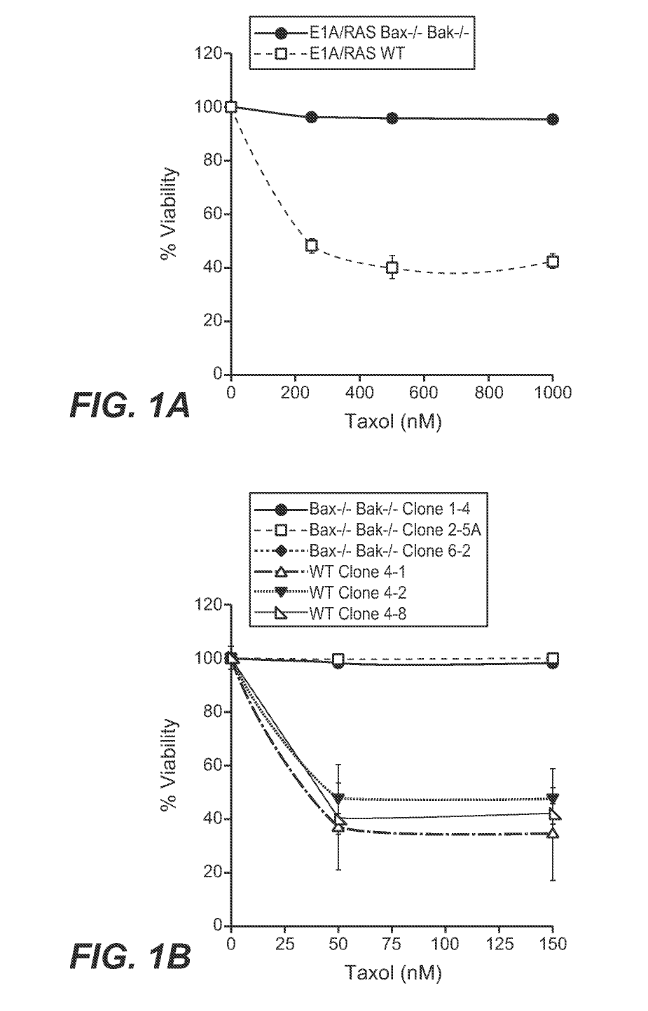

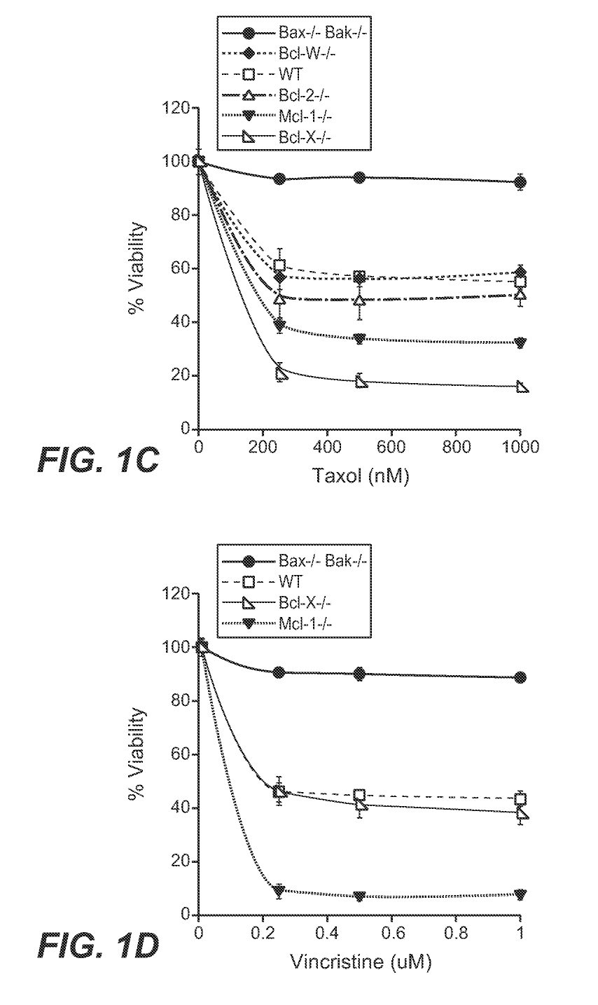

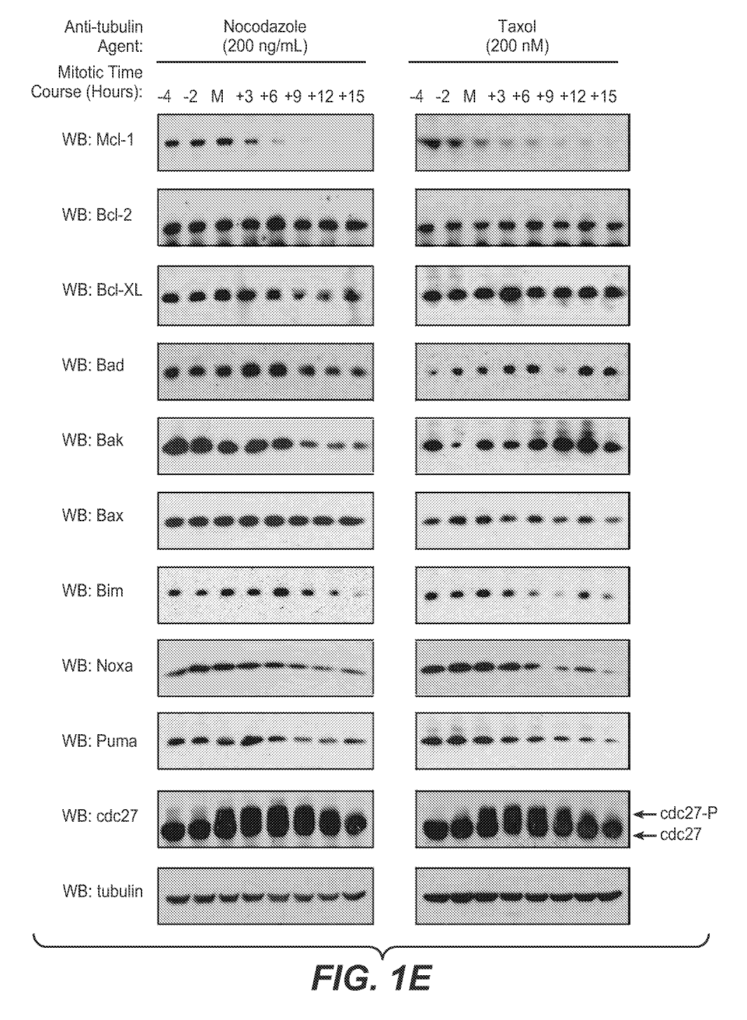

Aberrant expression of pro-survival Bcl-2 proteins promotes tumorigenesis and resistance to chemotherapeutics (Youle, R. J. and Strasser, A. (2008) Nat Rev Mol Cell Biol 9:47-59). Multiple lineages of BAX^{-/-}/BAK^{-/-} murine embryonic fibroblasts (MEFs) were resistant to killing by paclitaxel (TAXOL®) or nocodazole, whereas wild-type MEFs were significantly more sensitive (FIG. 1A, S2(A-E)). Nocodazole is an anti-neoplastic agent which exerts its effect in cells by interfering with the polymerization of microtubules. Cell death induced by antimitotic agents was confirmed in myeloid cells (FIG. 1B). As the Inhibitor of Apoptosis (IAP) proteins (Varfolomeev, E. and Vucic, D. (2008) Cell cycle (Georgetown, Tex. 7:1511-1521) do not play any role (FIG. S3), these results show Bcl-2 family proteins are key regulators of antimitotic-induced cell death in diverse cell types.

Expression levels of Mcl-1 and FBW7 are measured by immunohistochemistry (IHC) copy number analysis, or ELISA assays (Wertz et al (2011) Nature 471:110-114 which is incorporated by reference in its entirety). Mutations of Mcl-1 and FBW7 are detected by PCR methods. Measuring copy number for Mcl-1 and FBW7 is described in the methods of the Examples. Sequencing Mcl-1 and FBW7 is described in Kan et al (2010) Nature Aug. 12; 466(7308):869-73 and Peters et al (2007) Nat Methods Sep. 4; (9):713-5.

### Anti-Tubulin Chemotherapeutic Agents

Examples of anti-tubulin chemotherapeutic agents include, but are not limited to, paclitaxel (TAXOL®), docetaxel (TAXOTERE®), vincristine, vinblastine, vinorelbine (NAVELBINE®), eribulin (HALAVEN®), combretastatin, maytansines, dolastatins, auristatins, and the antibody-drug conjugates thereof.

Paclitaxel (TAXOL®, Bristol-Myers Squibb Oncology, Princeton N.J., CAS Reg. No. 33069-62-4) is isolated from the bark of the Pacific yew tree, Taxus brevifolia, and used to treat lung, ovarian, breast cancer, and advanced forms of Kaposi's sarcoma (Wani et al (1971) J. Am. Chem. Soc. 93:2325; Mekhail et al (2002) Expert. Opin. Pharmacother. 3:755-766). Paclitaxel is named as β-(benzoylamino)-α-hydroxy-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b) oxet-9-ylester,(2aR-(2a-α,4-β,4a-β,6-β,9-α(α-R*,β-S*),11-α,12-α,12α-α,2b-α))-benzenepropanoic acid, and has the structure:

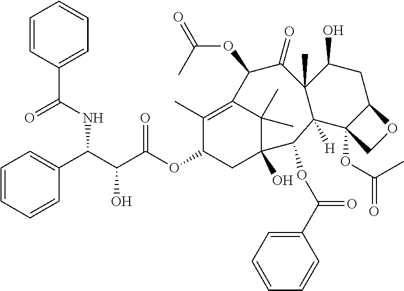

Vincristine (22-Oxovincaleukoblastine; leurocristine, VCR, LCR sulfate form: Vincristine sulfate, Kyocristine, ONCOVIN® (Lilly), Vincosid, Vincrex, CAS Reg. No. 57-22-7), is a vinca alkaloid from the Madagascar periwinkle Catharanthus roseus, formerly Vinca rosea (Johnson et al (1963) Cancer Res. 23:1390-1427; Neuss et al (1964) J. Am. Chem. Soc. 86:1440). Along with semisynthetic derivatives, vindesine and vinorelbine (NAVELBINE®), vincristine inhibits mitosis in metaphase by binding to tubulin and preventing the cell from making spindles necessary to move chromosomes as the cell divides. Vincristine is a chemotherapy drug that is given as a treatment for some types of cancer including leukemia, lymphoma, breast and lung cancer. Vincristine (leurocristine, VCR) is most effective in treating childhood leukemias and non-Hodgkin's lymphomas, where vinblastine (vincaleukoblastine, VLB) is used to treat Hodgkin's disease. Vincristine (CAS number 57-22-7) has the structure:

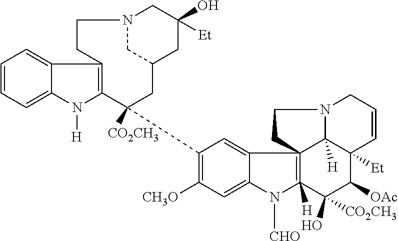

Docetaxel (TAXOTERE®, Sanofi-Aventis) is used to treat breast, ovarian, and NSCLC cancers (U.S. Pat. No. 4,814,470; U.S. Pat. No. 5,438,072; U.S. Pat. No. 5,698,582; U.S. Pat. No. 5,714,512; U.S. Pat. No. 5,750,561; Mangatal et al (1989) Tetrahedron 45:4177; Ringel et al (1991) J. Natl. Cancer Inst. 83:288; Bissery et al (1991) Cancer Res. 51:4845; Herbst et al (2003) Cancer Treat. Rev. 29:407-415; Davies et al (2003) Expert. Opin. Pharmacother. 4:553-565). Docetaxel is named as (2R,3S)-N-carboxy-3-phenylisoserine, N-tert-butyl ester, 13-ester with 5,20-epoxy-1,2,4,7,10,13-hexahydroxytax-11-en-9-one 4-acetate 2-benzoate, trihydrate (U.S. Pat. No. 4,814,470; EP 253738; CAS Reg. No. 114977-28-5) and has the structure:

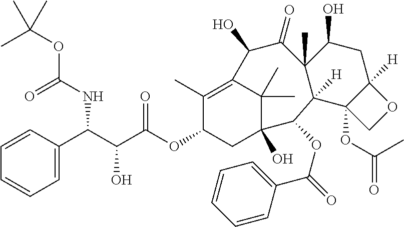

### Antibody-Drug Conjugates

Examples of anti-tubulin chemotherapeutic agents include antibody-drug conjugate (ADC) compounds where an anti-tubulin chemotherapeutic drug moiety is covalently attached to an antibody which targets a tumor cell.

An exemplary embodiment of an antibody-drug conjugate (ADC) compound comprises an antibody (Ab), and an anti-tubulin drug moiety (D), and a linker moiety (L) that attaches Ab to D. The antibody is attached through the one or more amino acid residues, such as lysine and cysteine, by the linker moiety (L) to D; the composition having Formula I:

Ab-(L-D)ₚ I

where p is 1 to about 20. The number of drug moieties which may be conjugated via a reactive linker moiety to an antibody molecule may be limited by the number of free cysteine residues, which are introduced by the methods described herein. Exemplary ADC of Formula I therefore comprise antibodies which have 1, 2, 3, or 4 engineered cysteine amino acids (Lyon, R. et al (2012) Methods in Enzym. 502:123-138).

The ADC compounds of the invention include those with anticancer activity. In an exemplary embodiment, the ADC compounds include a cysteine-engineered antibody conjugated, i.e. covalently attached by a linker, to the anti-tubulin drug moiety. The biological activity of the drug moiety is modulated by conjugation to an antibody. The antibody-drug conjugates (ADC) of the invention selectively deliver an effective dose of a the anti-tubulin drug to tumor tissue whereby greater selectivity, i.e. a lower efficacious dose, may be achieved.

### Antibodies

Antibodies which may be useful in anti-tubulin ADC in the methods of the invention include, but are not limited to, antibodies against cell surface receptors and tumor-associated antigens (TAA). Such antibodies may be used as naked antibodies (unconjugated to a drug or label moiety) or as Formula I antibody-drug conjugates (ADC). Tumor-associated antigens are known in the art, and can prepared for use in generating antibodies using methods and information which are well known in the art. In attempts to discover effective cellular targets for cancer diagnosis and therapy, researchers have sought to identify transmembrane or otherwise tumor-associated polypeptides that are specifically expressed on the surface of one or more particular type(s) of cancer cell as compared to on one or more normal non-cancerous cell(s). Often, such tumor-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies.

Examples of TAA include, but are not limited to, TAA (1)-(36) listed below. For convenience, information relating to these antigens, all of which are known in the art, is listed below and includes names, alternative names, Genbank accession numbers and primary reference(s), following nucleic acid and protein sequence identification conventions of the National Center for Biotechnology Information (NCBI). Nucleic acid and protein sequences corresponding to TAA (1)-(36) are available in public databases such as GenBank. Tumor-associated antigens targeted by antibodies include all amino acid sequence variants and isoforms possessing at least about 70%, 80%, 85%, 90%, or 95% sequence identity relative to the sequences identified in the cited references, or which exhibit substantially the same biological properties or characteristics as a TAA having a sequence found in the cited references. For example, a TAA having a variant sequence generally is able to bind specifically to an antibody that binds specifically to the TAA with the corresponding sequence listed. The disclosures in the references specifically recited herein are expressly incorporated by reference.

### Tumor-Associated Antigens (1)-(36):

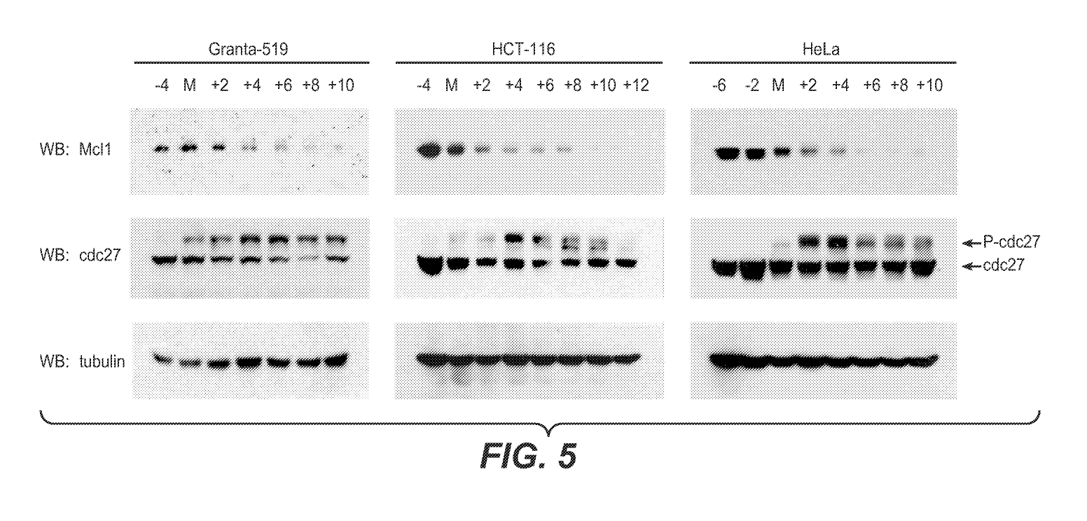

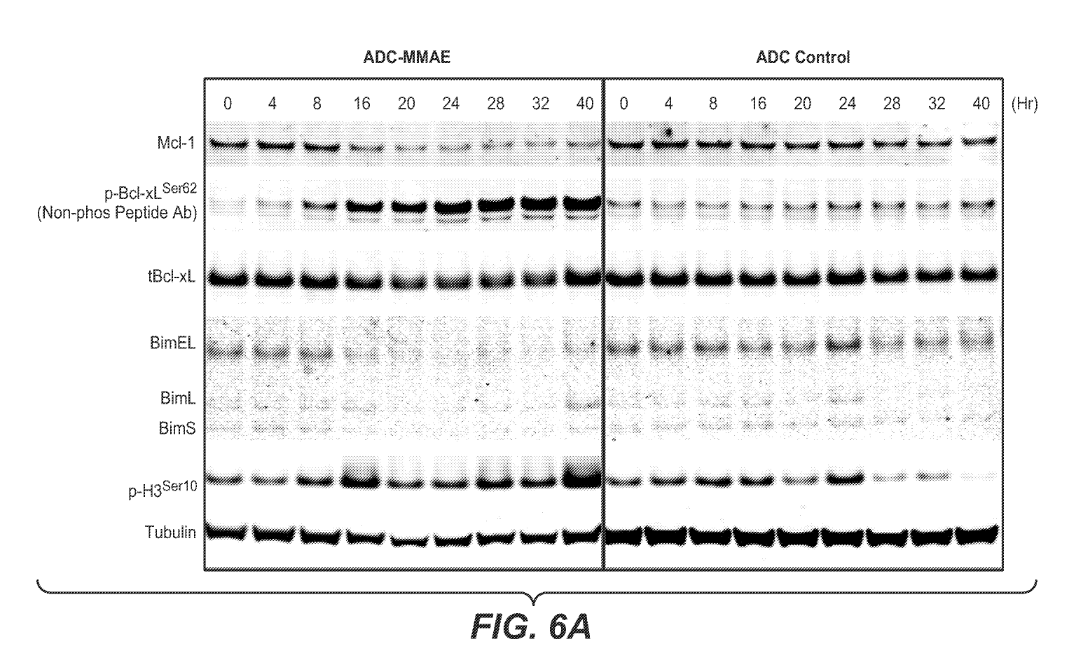

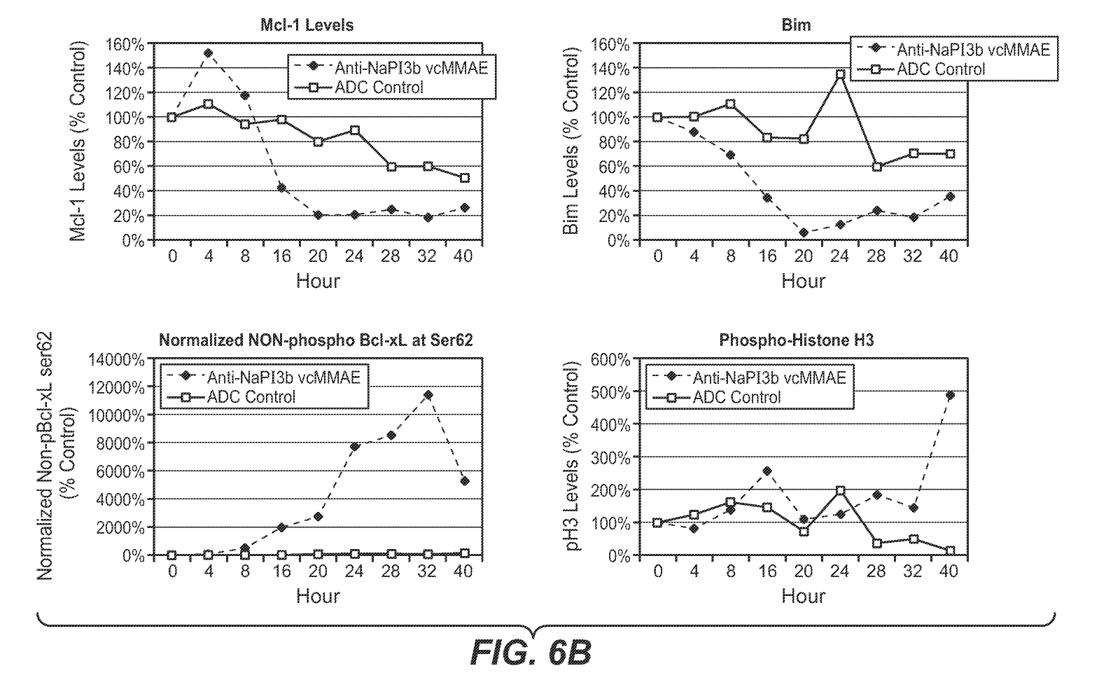

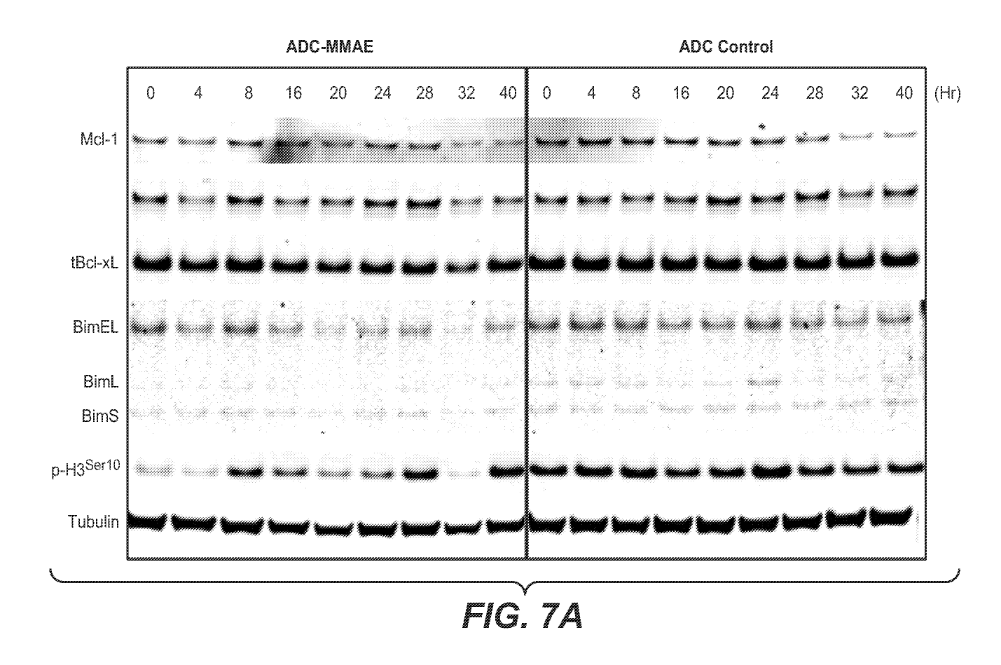

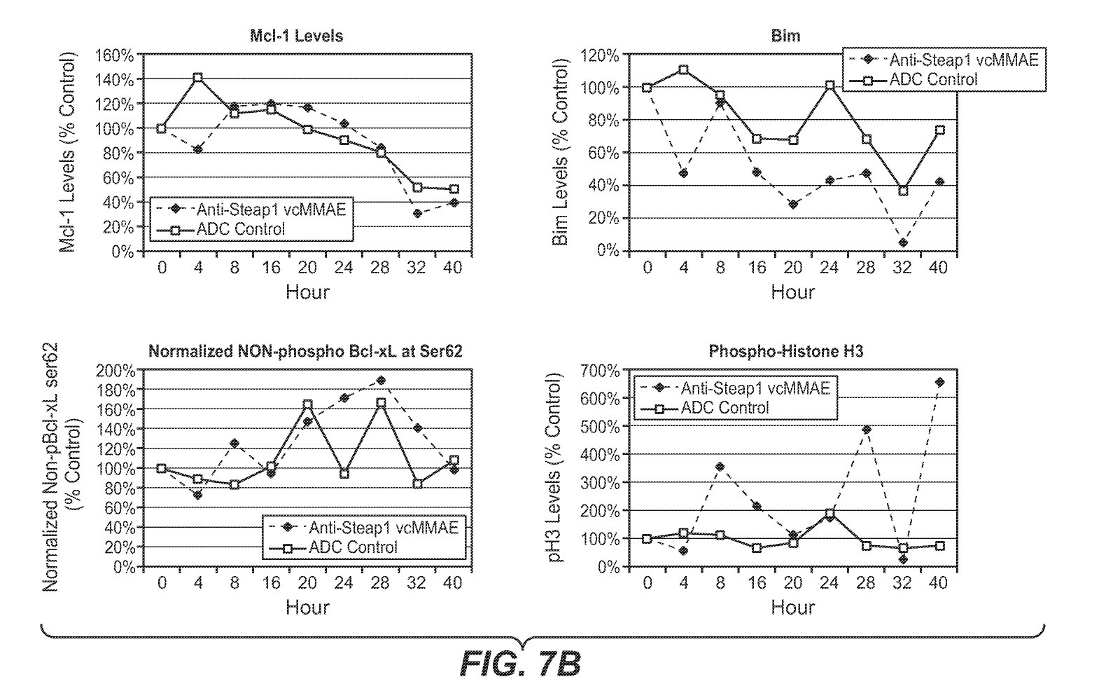

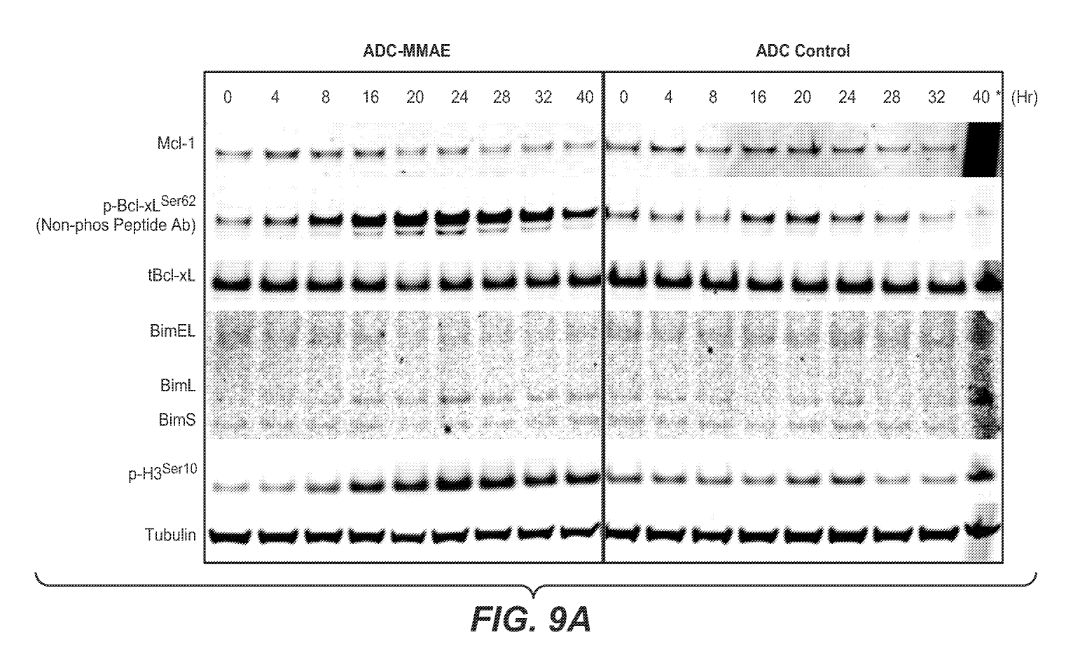

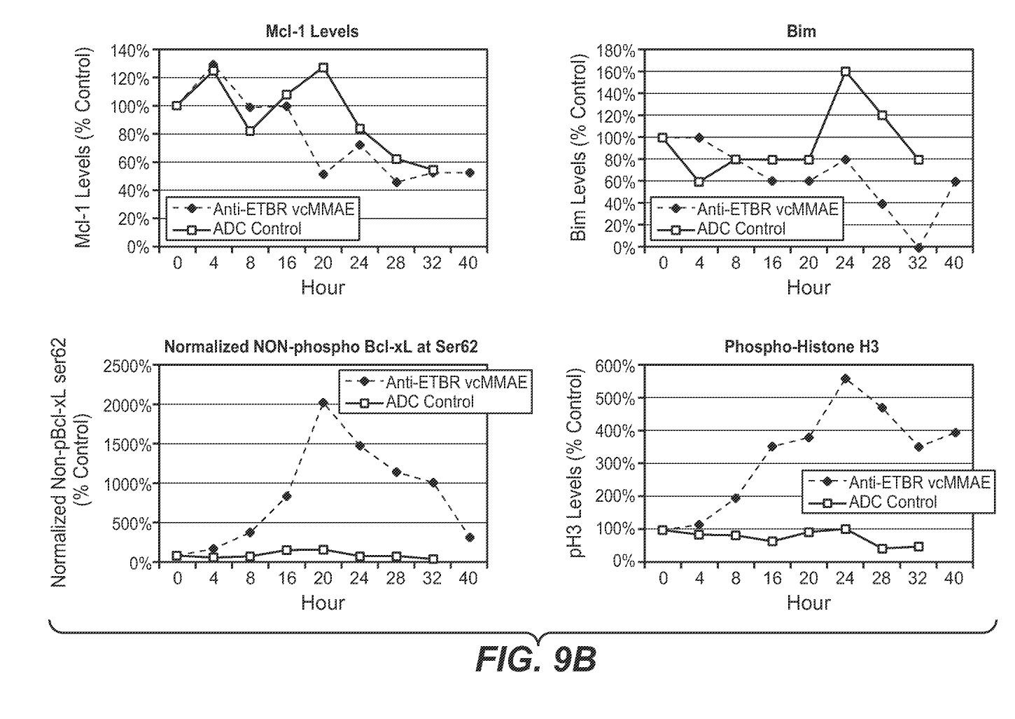

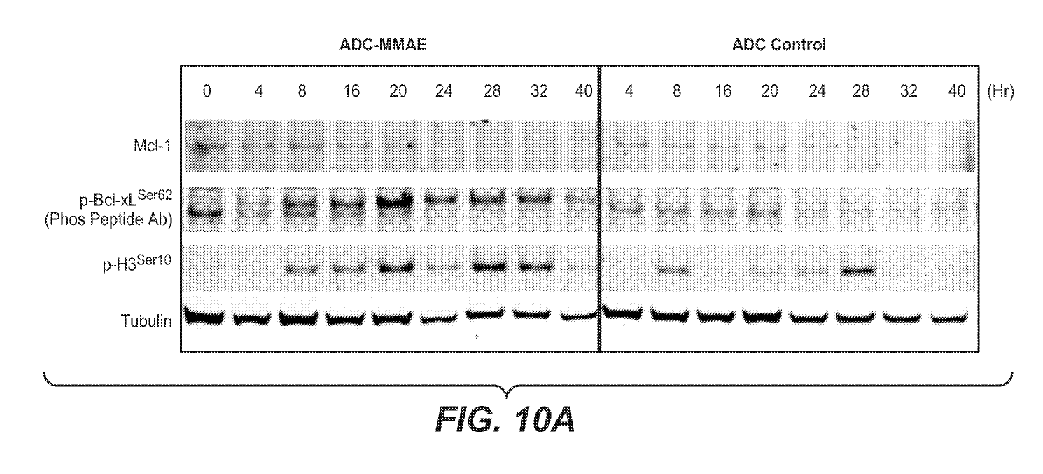

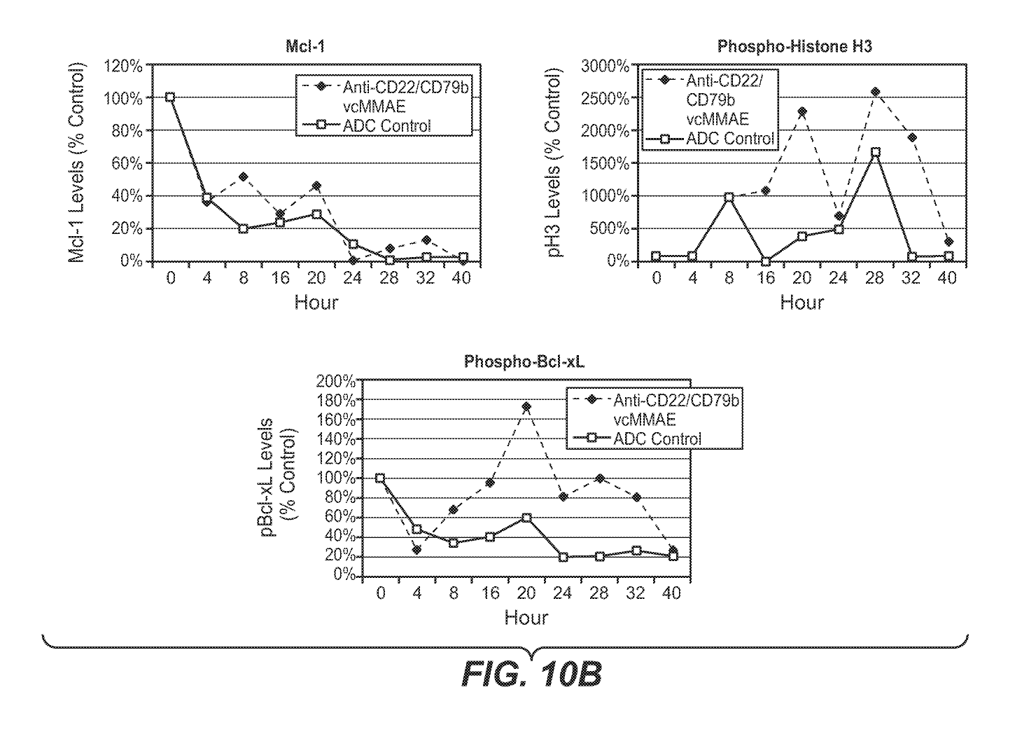

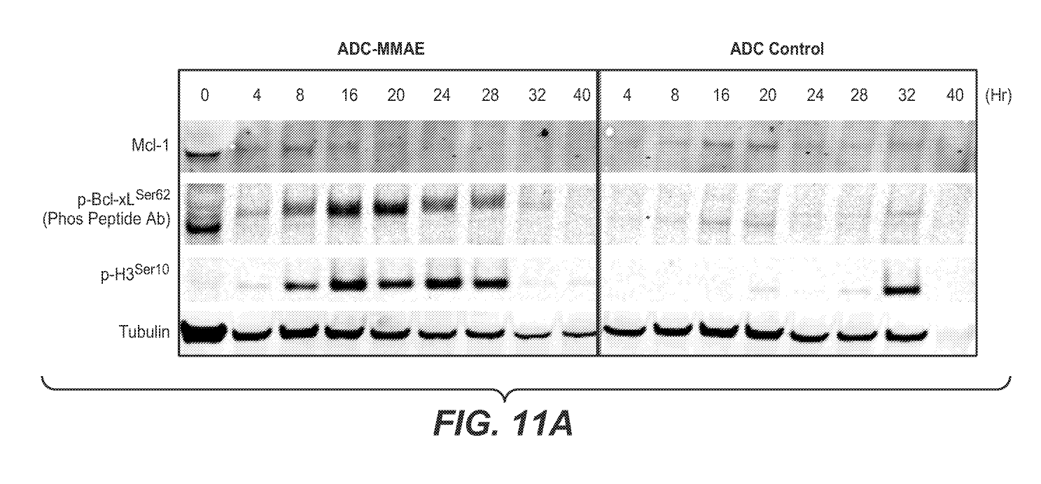

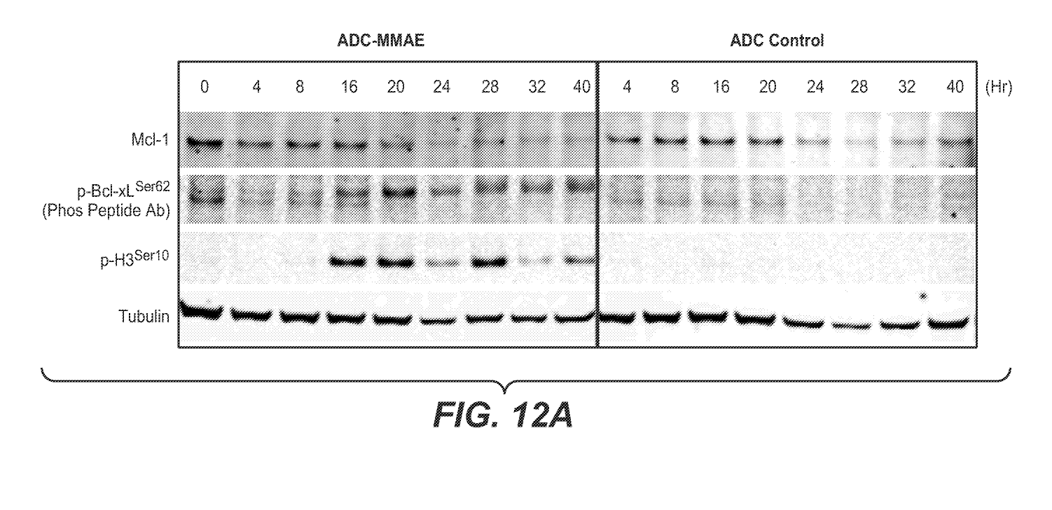

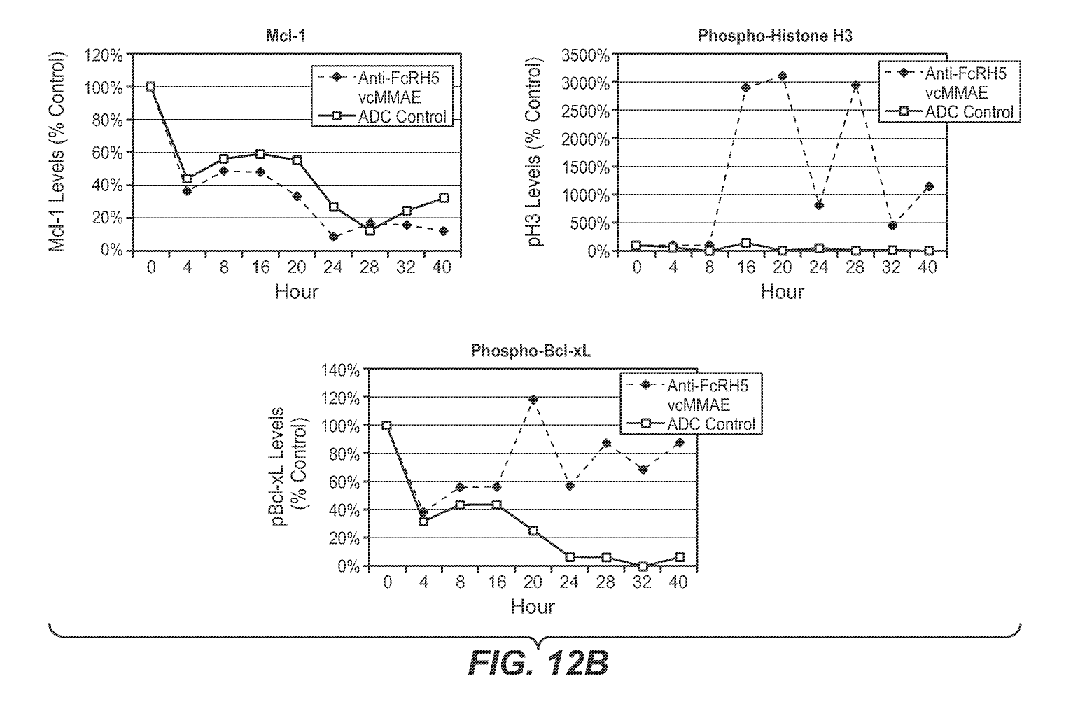

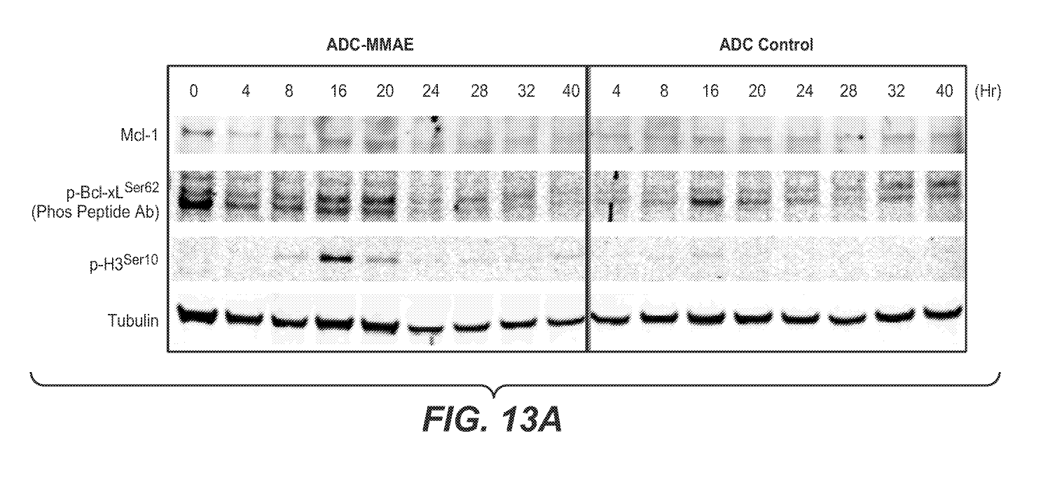

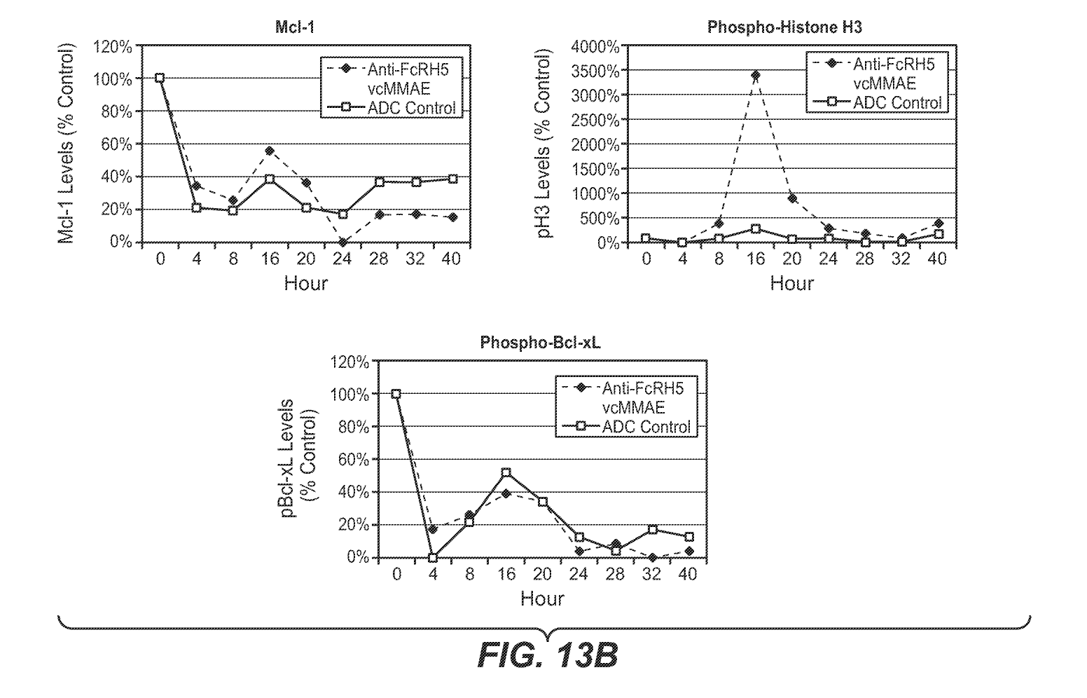

(1) BMPR1B (bone morphogenetic protein receptor-type IB, Genbank accession no. NM_001203) ten Dijke, P., et al Science 264 (5155):101-104 (1994), Oncogene 14 (11):1377-1382 (1997)); WO2004063362 (claim 2); WO2003042661 (claim 12); U52003134790-A1 (Page 38-39); WO2002102235 (claim 13; Page 296); WO2003055443 (Page 91-92); WO200299122 (Example 2; Page 528-530); WO2003029421 (claim 6); WO2003024392 (claim 2; FIG. 112); WO200298358 (claim 1; Page 183); WO200254940 (Page 100-101); WO200259377(Page 349-350); WO200230268 (claim 27; Page 376); WO200148204 (Example; FIG. 4); NP_001194 bone morphogenetic protein receptor, type IB/pid=NP_001194.1. Cross-references: MIM:603248; NP_001194.1; AY065994
(2) E16 (LAT1, SLC7A5, Genbank accession no. NM_003486) Biochem. Biophys. Res. Commun. 255 (2), 283-288 (1999), Nature 395 (6699):288-291 (1998), Gaugitsch, H. W., et al (1992) J. Biol. Chem. 267 (16):11267-11273); WO2004048938 (Example 2); WO2004032842 (Example IV); WO2003042661 (claim 12); WO2003016475 (claim 1); WO200278524 (Example 2); WO200299074 (claim 19; Page 127-129); WO200286443 (claim 27; Pages 222, 393); WO2003003906 (claim 10; Page 293); WO200264798 (claim 33; Page 93-95); WO200014228 (claim 5; Page 133-136); US2003224454 (FIG. 3); WO2003025138 (claim 12; Page 150); NP_003477 solute carrier family 7 (cationic amino acid transporter, y+system), member 5/pid=NP_003477.3—Homo sapiens; Cross-references: MIM:600182; NP_003477.3; NM_015923; NM_003486_1
(3) STEAP1 (six transmembrane epithelial antigen of prostate, Genbank accession no. NM_012449); Cancer Res. 61 (15), 5857-5860 (2001), Hubert, R. S., et al (1999) Proc. Natl. Acad. Sci. U.S.A. 96 (25):14523-14528); WO2004065577 (claim 6); WO2004027049 (FIG. 1L); EP1394274 (Example 11); WO2004016225 (claim 2); WO2003042661 (claim 12); US2003157089 (Example 5); US2003185830 (Example 5); US2003064397 (FIG. 2); WO200289747 (Example 5; Page 618-619); WO2003022995 (Example 9; FIG. 13A, Example 53; Page 173, Example 2; FIG. 2A); NP_036581 six transmembrane epithelial antigen of the prostate. Cross-references: MIM:604415; NP_036581.1; NM_012449_1
(4) 0772P (CA125, MUC16, Genbank accession no. AF361486); J. Biol. Chem. 276 (29):27371-27375 (2001)); WO2004045553 (claim 14); WO200292836 (claim 6; FIG. 12); WO200283866 (claim 15; Page 116-121); US2003124140 (Example 16); Cross-references: GI:34501467; AAK74120.3; AF361486_1
(5) MPF (MPF, MSLN, SMR, megakaryocyte potentiating factor, mesothelin, Genbank accession no. NM_005823) Yamaguchi, N., et al Biol. Chem. 269 (2), 805-808 (1994), Proc. Natl. Acad. Sci. U.S.A. 96 (20):11531-11536 (1999), Proc. Natl. Acad. Sci. U.S.A. 93 (1):136-140 (1996), J. Biol. Chem. 270 (37):21984-21990 (1995)); WO2003101283 (claim 14); (WO2002102235 (claim 13; Page 287-288); WO2002101075 (claim 4; Page 308-309); WO200271928 (Page 320-321); WO9410312 (Page 52-57); Cross-references: MIM:601051; NP_005814.2; NM_005823_1
(6) Napi3b (NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Feild, J. A., et al (1999) Biochem. Biophys. Res. Commun. 258 (3):578-582); WO2004022778 (claim 2); EP1394274 (Example 11); WO2002102235 (claim 13; Page 326); EP875569 (claim 1; Page 17-19); WO200157188 (claim 20; Page 329); WO2004032842 (Example IV); WO200175177 (claim 24; Page 139-140); Cross-references: MIM:604217; NP_006415.1; NM_006424_1
(7) Sema 5b (FLJ10372, KIAA1445, Mm.42015, SEMA5B, SEMAG, Semaphorin 5b Hlog, sema domain, seven thrombospondin repeats (type 1 and type 1-like), transmembrane domain (TM) and short cytoplasmic domain, (semaphorin) 5B, Genbank accession no. AB040878); Nagase T., et al (2000) DNA Res. 7 (2):143-150); WO2004000997 (claim 1); WO2003003984 (claim 1); WO200206339 (claim 1; Page 50); WO200188133 (claim 1; Page 41-43, 48-58); WO2003054152 (claim 20); WO2003101400 (claim 11); Accession: Q9P283; EMBL; AB040878; BAA95969.1. Genew; HGNC:10737
(8) PSCA hlg (2700050C12Rik, C530008O16Rik, RIKEN cDNA 2700050C12, RIKEN cDNA 2700050C12 gene, Genbank accession no. AY358628); Ross et al (2002) Cancer Res. 62:2546-2553; US2003129192 (claim 2); US2004044180 (claim 12); US2004044179 (claim 11); US2003096961 (claim 11); US2003232056 (Example 5); WO2003105758 (claim 12); US2003206918 (Example 5); EP1347046 (claim 1); WO2003025148 (claim 20); Cross-references: GI:37182378; AAQ88991.1; AY358628_1
(9) ETBR (Endothelin type B receptor, Genbank accession no. AY275463); Nakamuta M., et al Biochem. Biophys. Res. Commun. 177, 34-39, 1991; Ogawa Y., et al Biochem. Biophys. Res. Commun. 178, 248-255, 1991; Arai H., et al Jpn. Circ. J. 56, 1303-1307, 1992; Arai H., et al J. Biol. Chem. 268, 3463-3470, 1993; Sakamoto A., Yanagisawa M., et al Biochem. Biophys. Res. Commun. 178, 656-663, 1991; Elshourbagy N. A., et al J. Biol. Chem. 268, 3873-3879, 1993; Haendler B., et al J. Cardiovasc. Pharmacol. 20, s1-S4, 1992; Tsutsumi M., et al Gene 228, 43-49, 1999; Strausberg R. L., et al Proc. Natl. Acad. Sci. U.S.A. 99, 16899-16903, 2002; Bourgeois C., et al J. Clin. Endocrinol. Metab. 82, 3116-3123, 1997; Okamoto Y., et al Biol. Chem. 272, 21589-21596, 1997; Verheij J. B., et al Am. J. Med. Genet. 108, 223-225, 2002; Hofstra R. M. W., et al Eur. J. Hum. Genet. 5, 180-185, 1997; Puffenberger E. G., et al Cell 79, 1257-1266, 1994; Attie T., et al, Hum. Mol. Genet. 4, 2407-2409, 1995; Auricchio A., et al Hum. Mol. Genet. 5:351-354, 1996; Amiel J., et al Hum. Mol. Genet. 5, 355-357, 1996; Hofstra R. M. W., et al Nat. Genet. 12, 445-447, 1996; Svensson P. J., et al Hum. Genet. 103, 145-148, 1998; Fuchs S., et al Mol. Med. 7, 115-124, 2001; Pingault V., et al (2002) Hum. Genet. 111, 198-206; WO2004045516 (claim 1); WO2004048938 (Example 2); WO2004040000 (claim 151); WO2003087768 (claim 1); WO2003016475 (claim 1); WO2003016475 (claim 1); WO200261087 (FIG. 1); WO2003016494 (FIG. 6); WO2003025138 (claim 12; Page 144); WO200198351 (claim 1; Page 124-125); EP522868 (claim 8; FIG. 2); WO200177172 (claim 1; Page 297-299); US2003109676; U.S. Pat. No. 6,518,404 (FIG. 3); U.S. Pat. No. 5,773,223 (Claim 1a; Col 31-34); WO2004001004
(10) MSG783 (RNF124, hypothetical protein FLJ20315, Genbank accession no. NM_017763); WO2003104275 (claim 1); WO2004046342 (Example 2); WO2003042661 (claim 12); WO2003083074 (claim 14; Page 61); WO2003018621 (claim 1); WO2003024392 (claim 2; FIG. 93); WO200166689 (Example 6); Cross-references: LocusID:54894; NP_060233.2; NM_017763_1
(11) STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138); Lab. Invest. 82 (11):1573-1582 (2002)); WO2003087306; US2003064397 (claim 1; FIG. 1); WO200272596 (claim 13; Page 54-55); WO200172962 (claim 1; FIG. 4B); WO2003104270 (claim 11); WO2003104270 (claim 16); US2004005598 (claim 22); WO2003042661 (claim 12); US2003060612 (claim 12; FIG. 10); WO200226822 (claim 23; FIG. 2); WO200216429 (claim 12; FIG. 10); Cross-references: GI:22655488; AAN04080.1; AF455138_1
(12) TrpM4 (BR22450, FLJ20041, TRPM4, TRPM4B, transient receptor potential cation channel, subfamily M, member 4, Genbank accession no. NM_017636); Xu, X. Z., et al Proc. Natl. Acad. Sci. U.S.A. 98 (19):10692-10697 (2001), Cell 109 (3):397-407 (2002), J. Biol. Chem. 278 (33):30813-30820 (2003)); US2003143557 (claim 4); WO200040614 (claim 14; Page 100-103); WO200210382 (claim 1; FIG. 9A); WO2003042661 (claim 12); WO200230268 (claim 27; Page 391); US2003219806 (claim 4); WO200162794 (claim 14; FIG. 1A-D); Cross-references: MIM:606936; NP_060106.2; NM_017636_1
(13) CRIPTO (CR, CR1, CRGF, CRIPTO, TDGF1, teratocarcinoma-derived growth factor, Genbank accession no. NP_003203 or NM_003212); Ciccodicola, A., et al EMBO J. 8 (7):1987-1991 (1989), Am. J. Hum. Genet. 49 (3):555-565 (1991)); US2003224411 (claim 1); WO2003083041 (Example 1); WO2003034984 (claim 12); WO200288170 (claim 2; Page 52-53); WO2003024392 (claim 2; FIG. 58); WO200216413 (claim 1; Page 94-95, 105); WO200222808 (claim 2; FIG. 1); U.S. Pat. No. 5,854,399 (Example 2; Col 17-18); U.S. Pat. No. 5,792,616 (FIG. 2); Cross-references: MIM:187395; NP_003203.1; NM_003212_1
(14) CD21 (CR2 (Complement receptor 2) or C3DR (C3d/Epstein Barr virus receptor) or Hs.73792 Genbank accession no. M26004); Fujisaku et al (1989) J. Biol. Chem. 264 (4):2118-2125); Weis J. J., et al J. Exp. Med. 167, 1047-1066, 1988; Moore M., et al Proc. Natl. Acad. Sci. U.S.A. 84, 9194-9198, 1987; Barel M., et al Mol. Immunol. 35, 1025-1031, 1998; Weis J. J., et al Proc. Natl. Acad. Sci. U.S.A. 83, 5639-5643, 1986; Sinha S. K., et al (1993) J. Immunol. 150, 5311-5320; WO2004045520 (Example 4); US2004005538 (Example 1); WO2003062401 (claim 9); WO2004045520 (Example 4); WO9102536 (FIGS. 9.1-9.9); WO2004020595 (claim 1); Accession: P20023; Q13866; Q14212; EMBL; M26004; AAA35786.1.
(15) CD79b (CD79B, CD79β, IGb (immunoglobulin-associated beta), B29, Genbank accession no. NM_000626 or 11038674); Proc. Natl. Acad. Sci. U.S.A. (2003) 100 (7):4126-4131, Blood (2002) 100 (9):3068-3076, Muller et al (1992) Eur. J. Immunol. 22 (6):1621-1625); WO2004016225 (claim 2, FIG. 140); WO2003087768, US2004101874 (claim 1, page 102); WO2003062401 (claim 9); WO200278524 (Example 2); US2002150573 (claim 5, page 15); U.S. Pat. No. 5,644,033; WO2003048202 (claim 1, pages 306 and 309); WO 99/558658, U.S. Pat. No. 6,534,482 (claim 13, FIG. 17A/B); WO200055351 (claim 11, pages 1145-1146); Cross-references: MIM:147245; NP_000617.1; NM_000626_1
(16) FcRH2 (IFGP4, IRTA4, SPAP1A (SH2 domain containing phosphatase anchor protein 1a), SPAP1B, SPAP1C, Genbank accession no. NM_030764, AY358130); Genome Res. 13 (10):2265-2270 (2003), Immunogenetics 54 (2):87-95 (2002), Blood 99 (8):2662-2669 (2002), Proc. Natl. Acad. Sci. U.S.A. 98 (17):9772-9777 (2001), Xu, M. J., et al (2001) Biochem. Biophys. Res. Commun. 280 (3):768-775; WO2004016225 (claim 2); WO2003077836; WO200138490 (claim 5; FIG. 18D-1-18D-2); WO2003097803 (claim 12); WO2003089624 (claim 25); Cross-references: MIM:606509; NP_110391.2; NM_030764_1
(17) HER2 (ErbB2, Genbank accession no. M11730); Coussens L., et al Science (1985) 230(4730):1132-1139); Yamamoto T., et al Nature 319, 230-234, 1986; Semba K., et al Proc. Natl. Acad. Sci. U.S.A. 82, 6497-6501, 1985; Swiercz J. M., et al J. Cell Biol. 165, 869-880, 2004; Kuhns J. J., et al J. Biol. Chem. 274, 36422-36427, 1999; Cho H.-S., et al Nature 421, 756-760, 2003; Ehsani A., et al (1993) Genomics 15, 426-429; WO2004048938 (Example 2); WO2004027049 (FIG. 1I); WO2004009622; WO2003081210; WO2003089904 (claim 9); WO2003016475 (claim 1); US2003118592; WO2003008537 (claim 1); WO2003055439 (claim 29; FIG. 1A-B); WO2003025228 (claim 37; FIG. 5C); WO200222636 (Example 13; Page 95-107); WO200212341 (claim 68; FIG. 7); WO200213847 (Page 71-74); WO200214503 (Page 114-117); WO200153463 (claim 2; Page 41-46); WO200141787 (Page 15); WO200044899 (claim 52; FIG. 7); WO200020579 (claim 3; FIG. 2); U.S. Pat. No. 5,869,445 (claim 3; Col 31-38); WO9630514 (claim 2; Page 56-61); EP1439393 (claim 7); WO2004043361 (claim 7); WO2004022709; WO200100244 (Example 3; FIG. 4); Accession: P04626; EMBL; M11767; AAA35808.1. EMBL; M11761; AAA35808.1
(18) NCA (CEACAM6, Genbank accession no. M18728); Barnett T., et al Genomics 3, 59-66, 1988; Tawaragi Y., et al Biochem. Biophys. Res. Commun. 150, 89-96, 1988; Strausberg R. L., et al Proc. Natl. Acad. Sci. U.S.A. 99:16899-16903, 2002; WO2004063709; EP1439393 (claim 7); WO2004044178 (Example 4); WO2004031238; WO2003042661 (claim 12); WO200278524 (Example 2); WO200286443 (claim 27; Page 427); WO200260317 (claim 2); Accession: P40199; Q14920; EMBL; M29541; AAA59915.1. EMBL; M18728
(19) MDP (DPEP1, Genbank accession no. BC017023); Proc. Natl. Acad. Sci. U.S.A. 99 (26):16899-16903 (2002)); WO2003016475 (claim 1); WO200264798 (claim 33; Page 85-87); JP05003790 (FIG. 6-8); WO9946284 (FIG. 9); Cross-references: MIM:179780; AAH17023.1; BC017023_1
(20) IL20Rα (IL20Rα, ZCYTOR7, Genbank accession no. AF184971); Clark H. F., et al Genome Res. 13, 2265-2270, 2003; Mungall A. J., et al Nature 425, 805-811, 2003; Blumberg H., et al Cell 104, 9-19, 2001; Dumoutier L., et al J. Immunol. 167, 3545-3549, 2001; Parrish-Novak J., et al J. Biol. Chem. 277, 47517-47523, 2002; Pletnev S., et al (2003) Biochemistry 42:12617-12624; Sheikh F., et al (2004) J. Immunol. 172, 2006-2010; EP1394274 (Example 11); US2004005320 (Example 5); WO2003029262 (Page 74-75); WO2003002717 (claim 2; Page 63); WO200222153 (Page 45-47); US2002042366 (Page 20-21); WO200146261 (Page 57-59); WO200146232 (Page 63-65); WO9837193 (claim 1; Page 55-59); Accession: Q9UHF4; Q6UWA9; Q96SH8; EMBL; AF184971; AAF01320.1.
(21) Brevican (BCAN, BEHAB, Genbank accession no. AF229053); Gary S. C., et al Gene 256, 139-147, 2000; Clark H. F., et al Genome Res. 13, 2265-2270, 2003; Strausberg R. L., et al Proc. Natl. Acad. Sci. U.S.A. 99, 16899-16903, 2002; US2003186372 (claim 11); US2003186373 (claim 11); US2003119131 (claim 1; FIG. 52); US2003119122 (claim 1; FIG. 52); US2003119126 (claim 1); US2003119121 (claim 1; FIG. 52); US2003119129 (claim 1); US2003119130 (claim 1); US2003119128 (claim 1; FIG. 52); US2003119125 (claim 1); WO2003016475 (claim 1); WO200202634 (claim 1)
(22) EphB2R (DRT, ERK, Hek5, EPHT3, Tyro5, Genbank accession no. NM_004442); Chan, J. and Watt, V. M., Oncogene 6 (6), 1057-1061 (1991) Oncogene 10 (5):897-905 (1995), Annu Rev. Neurosci. 21:309-345 (1998), Int. Rev. Cytol. 196:177-244 (2000)); WO2003042661 (claim 12); WO200053216 (claim 1; Page 41); WO2004065576 (claim 1); WO2004020583 (claim 9); WO2003004529 (Page 128-132); WO200053216 (claim 1; Page 42); Cross-references: MIM:600997; NP_004433.2; NM_004442_1
(23) ASLG659 (B7h, Genbank accession no. AX092328); US20040101899 (claim 2); WO2003104399 (claim 11); WO2004000221 (FIG. 3); US2003165504 (claim 1); US2003124140 (Example 2); US2003065143 (FIG. 60); WO2002102235 (claim 13; Page 299); US2003091580 (Example 2); WO200210187 (claim 6; FIG. 10); WO200194641 (claim 12; FIG. 7b); WO200202624 (claim 13; FIG. 1A-1B); US2002034749 (claim 54; Page 45-46); WO200206317 (Example 2; Page 320-321, claim 34; Page 321-322); WO200271928 (Page 468-469); WO200202587 (Example 1; FIG. 1); WO200140269 (Example 3; Pages 190-192); WO200036107 (Example 2; Page 205-207); WO2004053079 (claim 12); WO2003004989 (claim 1); WO200271928 (Page 233-234, 452-453); WO 0116318
(24) PSCA (Prostate stem cell antigen precursor, Genbank accession no. AJ297436); Reiter R. E., et al Proc. Natl. Acad. Sci. U.S.A. 95, 1735-1740, 1998; Gu Z., et al Oncogene 19, 1288-1296, 2000; Biochem. Biophys. Res. Commun. (2000) 275(3):783-788; WO2004022709; EP1394274 (Example 11); US2004018553 (claim 17); WO2003008537 (claim 1); WO200281646 (claim 1; Page 164); WO2003003906 (claim 10; Page 288); WO200140309 (Example 1; FIG. 17); US2001055751 (Example 1; FIG. 1b); WO200032752 (claim 18; FIG. 1); WO9851805 (claim 17; Page 97); WO9851824 (claim 10; Page 94); WO9840403 (claim 2; FIG. 1B); Accession: 043653; EMBL; AF043498; AAC39607.1
(25) GEDA (Genbank accession No. AY260763); AAP14954 lipoma HMGIC fusion-partner-like protein/pid=AAP14954.1 —Homo sapiens (human); WO2003054152 (claim 20); WO2003000842 (claim 1); WO2003023013 (Example 3, claim 20); US2003194704 (claim 45); Cross-references: GI:30102449; AAP14954.1; AY260763_1
(26) BAFF-R (B cell-activating factor receptor, BLyS receptor 3, BR3, Genbank accession No. AF116456); BAFF receptor/pid=NP_443177.1 —Homo sapiens: Thompson, J. S., et al Science 293 (5537), 2108-2111 (2001); WO2004058309; WO2004011611; WO2003045422 (Example; Page 32-33); WO2003014294 (claim 35; FIG. 6B); WO2003035846 (claim 70; Page 615-616); WO200294852 (Col 136-137); WO200238766 (claim 3; Page 133); WO200224909 (Example 3; FIG. 3); Cross-references: MIM:606269; NP_443177.1; NM_052945_1; AF132600
(27) CD22 (B-cell receptor CD22-B isoform, BL-CAM, Lyb-8, Lyb8, SIGLEC-2, FLJ22814, Genbank accession No. AK026467); Wilson et al (1991) J. Exp. Med. 173:137-146; WO2003072036 (claim 1; FIG. 1); Cross-references: MIM:107266; NP_001762.1; NM_001771_1
(28) CD79a (CD79A, CD79α, immunoglobulin-associated alpha, a B cell-specific protein that covalently interacts with Ig beta (CD79B) and forms a complex on the surface with Ig M molecules, transduces a signal involved in B-cell differentiation), pI: 4.84, MW: 25028 TM: 2 [P] Gene Chromosome: 19q13.2, Genbank accession No. NP_001774.10); WO2003088808, US20030228319; WO2003062401 (claim 9); US2002150573 (claim 4, pages 13-14); WO9958658 (claim 13, FIG. 16); WO9207574 (FIG. 1); U.S. Pat. No. 5,644,033; Ha et al (1992) J. Immunol. 148(5):1526-1531; Mueller et al (1992) Eur. J. Biochem. 22:1621-1625; Hashimoto et al (1994) Immunogenetics 40(4):287-295; Preud'homme et al (1992) Clin. Exp. Immunol. 90(1):141-146; Yu et al (1992) J. Immunol. 148(2) 633-637; Sakaguchi et al (1988) EMBO J. 7(11):3457-3464
(29) CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, pI: 8.54 MW: 41959 TM: 7 [P] Gene Chromosome: 11q23.3, Genbank accession No. NP_001707.1); WO2004040000; WO2004015426; US2003105292 (Example 2); U.S. Pat. No. 6,555,339 (Example 2); WO200261087 (FIG. 1); WO200157188 (claim 20, page 269); WO200172830 (pages 12-13); WO200022129 (Example 1, pages 152-153, Example 2, pages 254-256); WO9928468 (claim 1, page 38); U.S. Pat. No. 5,440,021 (Example 2, col 49-52); WO9428931 (pages 56-58); WO9217497 (claim 7, FIG. 5); Dobner et al (1992) Eur. J. Immunol. 22:2795-2799; Barella et al (1995) Biochem. J. 309:773-779
(30) HLA-DOB (Beta subunit of MHC class II molecule (Ia antigen) that binds peptides and presents them to CD4+ T lymphocytes); 273 aa, pI: 6.56, MW: 30820.TM: 1 [P] Gene Chromosome: 6p21.3, Genbank accession No. NP_002111.1); Tonnelle et al (1985) EMBO J. 4(11):2839-2847; Jonsson et al (1989) Immunogenetics 29(6):411-413; Beck et al (1992) J. Mol. Biol. 228:433-441; Strausberg et al (2002) Proc. Natl. Acad. Sci USA 99:16899-16903; Servenius et al (1987) J. Biol. Chem. 262:8759-8766; Beck et al (1996) J. Mol. Biol. 255:1-13; Naruse et al (2002) Tissue Antigens 59:512-519; WO9958658 (claim 13, FIG. 15); U.S. Pat. No. 6,153,408 (Col 35-38); U.S. Pat. No. 5,976,551 (col 168-170); U.S. Pat. No. 6,011,146 (col 145-146); Kasahara et al (1989) Immunogenetics 30(1):66-68; Larhammar et al (1985) J. Biol. Chem. 260(26):14111-14119
(31) P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), pI: 7.63, MW: 47206 TM: 1 [P] Gene Chromosome: 17p13.3, Genbank accession No. NP_002552.2); Le et al (1997) FEBS Lett. 418(1-2):195-199; WO2004047749; WO2003072035 (claim 10); Touchman et al (2000) Genome Res. 10:165-173; WO200222660 (claim 20); WO2003093444 (claim 1); WO2003087768 (claim 1); WO2003029277 (page 82)
(32) CD72 (B-cell differentiation antigen CD72, Lyb-2); 359 aa, pI: 8.66, MW: 40225, TM: 1 [P] Gene Chromosome: 9p13.3, Genbank accession No. NP_001773.1); WO2004042346 (claim 65); WO2003026493 (pages 51-52, 57-58); WO200075655 (pages 105-106); Von Hoegen et al (1990) J. Immunol. 144(12):4870-4877; Strausberg et al (2002) Proc. Natl. Acad. Sci USA 99:16899-16903.
(33) LY64 (Lymphocyte antigen 64 (RP105), type I membrane protein of the leucine rich repeat (LRR) family, regulates B-cell activation and apoptosis, loss of function is associated with increased disease activity in patients with systemic lupus erythematosis); 661 aa, pI: 6.20, MW: 74147 TM: 1 [P] Gene Chromosome: 5q12, Genbank accession No. NP_005573.1); US2002193567; WO9707198 (claim 11, pages 39-42); Miura et al (1996) Genomics 38(3):299-304; Miura et al (1998) Blood 92:2815-2822; WO2003083047; WO9744452 (claim 8, pages 57-61); WO200012130 (pages 24-26)
(34) FcRH1 (Fc receptor-like protein 1, a putative receptor for the immunoglobulin Fc domain that contains C2 type Ig-like and ITAM domains, may have a role in B-lymphocyte differentiation); 429 aa, pI: 5.28, MW: 46925 TM: 1 [P] Gene Chromosome: 1q21-1q22, Genbank accession No. NP_443170.1); WO2003077836; WO200138490 (claim 6, FIG. 18E-1-18-E-2); Davis et al (2001) Proc. Natl. Acad. Sci USA 98(17):9772-9777; WO2003089624 (claim 8); EP1347046 (claim 1); WO2003089624 (claim 7)
(35) IRTA2 (FcRH5, Fc-receptor homolog 5, Immunoglobulin superfamily receptor translocation associated 2, a putative immunoreceptor with possible roles in B cell development and lymphomagenesis; deregulation of the gene by translocation occurs in some B cell malignancies); 977 aa, pI: 6.88, MW: 106468, TM: 1 [P] Gene Chromosome: 1q21, Genbank accession No. Human: AF343662, AF343663, AF343664, AF343665, AF369794, AF397453, AK090423, AK090475, AL834187, AY358085; Mouse:AK089756, AY158090, AY506558; NP_112571.1; WO2003024392 (claim 2, FIG. 97); Ise et al (2007) Leukemia 21:169-174; Nakayama et al (2000) Biochem. Biophys. Res. Commun. 277(1):124-127; WO2003077836; WO200138490 (claim 3, FIG. 18B-1-18B-2)
(36) TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP_057276; NCBI Gene: 23671; OMIM: 605734; SwissProt Q9UIK5; Genbank accession No. AF179274; AY358907, CAF85723, CQ782436; WO2004074320; JP2004113151; WO2003042661; WO2003009814; EP1295944 (pages 69-70); WO200230268 (page 329); WO200190304; US2004249130; US2004022727; WO2004063355; US2004197325; US2003232350; US2004005563; US2003124579; Horie et al (2000) Genomics 67:146-152; Uchida et al (1999) Biochem. Biophys. Res. Commun. 266:593-602; Liang et al (2000) Cancer Res. 60:4907-12; Glynne-Jones et al (2001) Int J Cancer. Oct. 15; 94(2):178-84.

The antibody may also be a fusion protein comprising an albumin-binding peptide (ABP) sequence (Dennis et al (2002) J Biol Chem. 277:35035-35043 at Tables III and IV, page 35038; (ii) US 20040001827 at [0076]; and (iii) WO 01/45746 at pages 12-13).

### Anti-Tubulin Drug Moieties

The anti-tubulin drug moiety (D) of the antibody-drug conjugates (ADC) includes any compound, moiety or group that has a cytotoxic or cytostatic anti-tubulin effect. Drug moieties include chemotherapeutic agents, which may function as microtubulin inhibitors.

Exemplary drug moieties include, but are not limited to, a maytansinoid, an auristatin, a dolastatin, a taxane, a vinca alkaloid, and stereoisomers, isosteres, analogs or derivatives thereof.

Maytansine compounds suitable for use as maytansinoid drug moieties are well known in the art, and can be isolated from natural sources according to known methods, produced using genetic engineering techniques (see Yu et al (2002) Proc. Nat. Acad. Sci. (USA) 99:7968-7973), or maytansinol and maytansinol analogues prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions

Exemplary maytansinoid drug moieties also include those having modifications such as: C-9-SH (U.S. Pat. No. 4,424,219) (prepared by the reaction of maytansinol with H₂S or P₂S₅); C-14-alkoxymethyl(demethoxy/CH₂ OR)(U.S. Pat. No. 4,331,598); C-14-hydroxymethyl or acyloxymethyl(CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol). Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable.

The anti-tubulin drug moiety (D) of the antibody-drug conjugates (ADC) of Formula I include maytansinoids having the structure:

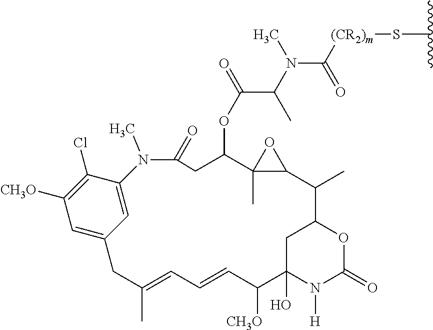

where the wavy line indicates the covalent attachment of the sulfur atom of D to a linker (L) of an antibody-drug conjugate (ADC). R may independently be H or a C₁-C₆ alkyl selected from methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. The alkylene chain attaching the amide group to the sulfur atom may be methanyl, ethanyl, or propyl, i.e. m is 1, 2, or 3.

Maytansine compounds inhibit cell proliferation by inhibiting the formation of microtubules during mitosis through inhibition of polymerization of the microtubulin protein, tubulin (Remillard et al (1975) Science 189:1002-1005). Maytansine and maytansinoids are highly cytotoxic but their clinical use in cancer therapy has been greatly limited by their severe systemic side-effects primarily attributed to their poor selectivity for tumors. Clinical trials with maytansine had been discontinued due to serious adverse effects on the central nervous system and gastrointestinal system (Issel et al (1978) Can. Treatment. Rev. 5:199-207).

Maytansinoid drug moieties are attractive anti-tubulin drug moieties in antibody-drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines (US 2005/0169933; WO 2005/037992; U.S. Pat. No. 5,208,020).

As with other drug moieties, all stereoisomers of the maytansinoid drug moiety are contemplated for the compounds of the invention, i.e. any combination of R and S configurations at the chiral carbons of D. In one embodiment, the maytansinoid drug moiety (D) will have the following stereochemistry:

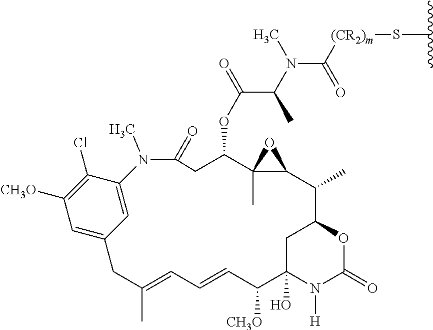

Exemplary embodiments of maytansinoid drug moieties include: DM1, (CR₂)ₘ=CH₂CH₂; DM3, (CR₂)ₘ=CH₂CH₂CH(CH₃); and DM4, (CR₂)ₘ=CH₂CH₂C(CH₃)₂ (Widdison et al (2006) 49:4292-4408), having the structures:

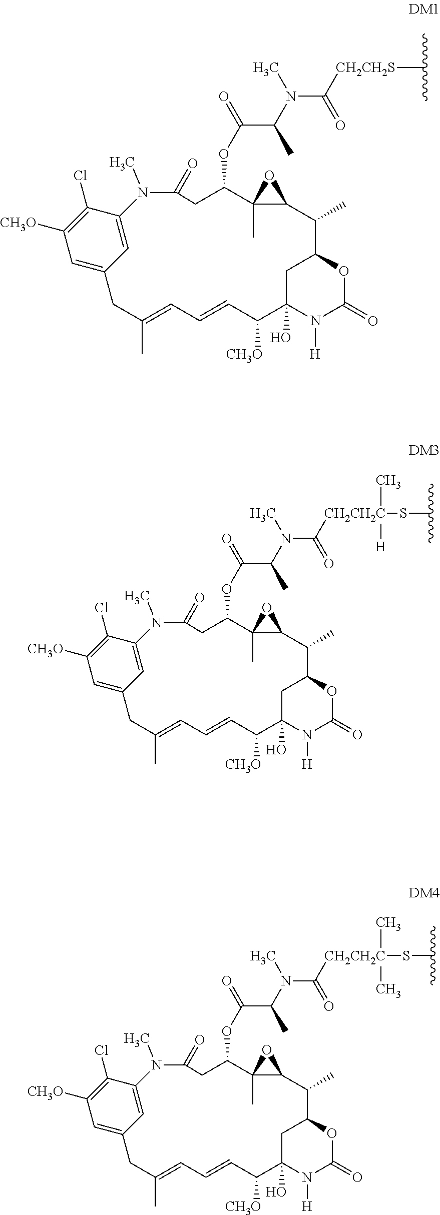

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

The anti-tubulin drug moiety (D) of the antibody-drug conjugates (ADC) of Formula I also include dolastatins and their peptidic analogs and derivatives, the auristatins (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). Various forms of a dolastatin or auristatin drug moiety may be covalently attached to an antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Drug moieties include dolastatins, auristatins (U.S. Pat. No. 5,635,483; U.S. Pat. No. 5,780,588; U.S. Pat. No. 5,767,237; U.S. Pat. No. 6,124,431), and analogs and derivatives thereof. Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties D_{E} and D_{F}, disclosed in U.S. Pat. No. 7,498,298 and U.S. Pat. No. 7,659,241, the disclosure of each which is expressly incorporated by reference in their entirety.

The drug moiety (D) of the antibody-drug conjugates (ADC) of Formula I include the monomethylauristatin drug moieties MMAE and MMAF linked through the N-terminus to the antibody, and having the structures:

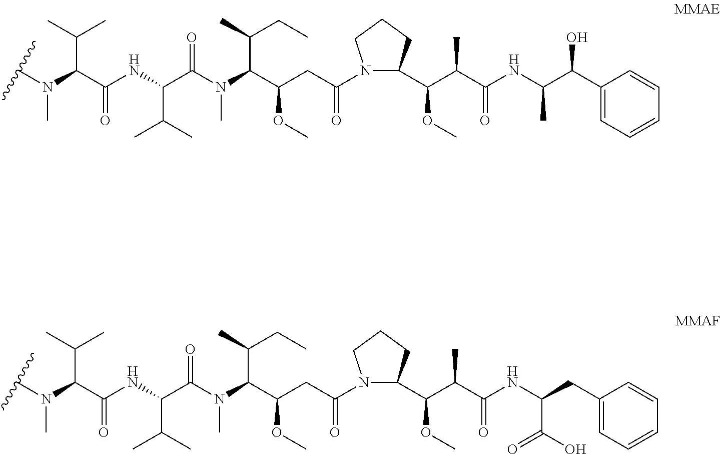 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

where the wavy line indicates the site of attachment to the linker (L).

MMAE (vedotin, (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide, CAS Reg. No. 474645-27-7) has the structure:

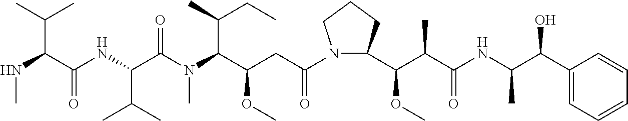 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Typically, peptide-based drug moieties can be prepared by forming a peptide bond between two or more amino acids and/or peptide fragments. Such peptide bonds can be prepared, for example, according to liquid phase or solid phase synthesis methods (see E. Schröder and K. Lübke, “The Peptides”, volume 1, pp 76-136, 1965, Academic Press) that are well known in the field of peptide chemistry.

Linkers

A “Linker” (L) is a bifunctional or multifunctional moiety which can be used to link one or more anti-tubulin Drug moieties (D) and an antibody unit (Ab) to form antibody-drug conjugates (ADC) of Formula I. Antibody-drug conjugates (ADC) can be conveniently prepared using a Linker having reactive functionality for binding to the Drug and to the Antibody. A cysteine thiol of a cysteine engineered antibody (Ab) can form a bond with a functional group of a linker reagent, a drug moiety or drug-linker intermediate.

In one aspect, a Linker has a reactive site which has an electrophilic group that is reactive to a nucleophilic cysteine present on an antibody. The cysteine thiol of the antibody is reactive with an electrophilic group on a Linker and forms a covalent bond to a Linker. Useful electrophilic groups include, but are not limited to, maleimide and haloacetamide groups.

Cysteine engineered antibodies react with linker reagents or drug-linker intermediates, with electrophilic functional groups such as maleimide or a-halo carbonyl, according to the conjugation method at page 766 of Klussman, et al (2004), Bioconjugate Chemistry 15(4):765-773, and according to the protocol of Example 4.

In yet another embodiment, the reactive group of a linker reagent or drug-linker intermediate contains a thiol-reactive functional group that can form a bond with a free cysteine thiol of an antibody. Examples of thiol-reaction functional groups include, but are not limited to, maleimide, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates.

In another embodiment, the linker may be a dendritic type linker for covalent attachment of more than one drug moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11:1761-1768; King (2002) Tetrahedron Letters 43:1987-1990). Dendritic linkers can increase the molar ratio of drug to antibody, i.e. loading, which is related to the potency of the ADC. Thus, where a cysteine engineered antibody bears only one reactive cysteine thiol group, a multitude of drug moieties may be attached through a dendritic linker.

The linker may comprise amino acid residues that link the antibody (Ab) to the drug moiety (D) of the cysteine engineered antibody-drug conjugate (ADC) of the invention. The amino acid residues may form a dipeptide, tripeptide, tetrapeptide, pentapeptide, hexapeptide, heptapeptide, octapeptide, nonapeptide, decapeptide, undecapeptide or dodecapeptide unit. Amino acid residues include those occurring naturally, as well as minor amino acids and non-naturally occurring amino acid analogs, such as citrulline.

Useful amino acid residue units can be designed and optimized in their selectivity for enzymatic cleavage by a particular enzymes, for example, a tumor-associated protease to liberate an active drug moiety. In one embodiment, an amino acid residue unit, such as valine-citrulline (vc or val-cit), is that whose cleavage is catalyzed by cathepsin B, C and D, or a plasmin protease.

A linker unit may be of the self-immolative type such as a para-aminobenzylcarbamoyl (PAB) unit where the ADC has the exemplary structure:

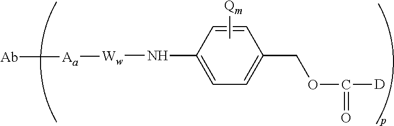

wherein Q is —C₁-C₈ alkyl, —O—(C₁-C₈ alkyl), -halogen, -nitro or -cyano; m is an integer ranging from 0-4; and p ranges from 1 to 4.

Other examples of self-immolative spacers include, but are not limited to, aromatic compounds that are electronically similar to the PAB group such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Elimination of amine-containing drugs that are substituted at glycine (Kingsbury et al (1984) J. Med. Chem. 27:1447) are also examples of self-immolative spacer useful in ADCs.

In another embodiment, linker L may be a dendritic type linker for covalent attachment of more than one drug moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where a cysteine engineered antibody bears only one reactive cysteine thiol group, a multitude of drug moieties may be attached through a dendritic linker (WO 2004/01993; Szalai et al (2003) J. Amer. Chem. Soc. 125:15688-15689; Shamis et al (2004) J. Amer. Chem. Soc. 126:1726-1731; Amir et al (2003) Angew. Chem. Int. Ed. 42:4494-4499).

Embodiments of the Formula Ia antibody-drug conjugate compounds include (val-cit), (MC-val-cit), and (MC-val-cit-PAB=MC-vc-PAB):

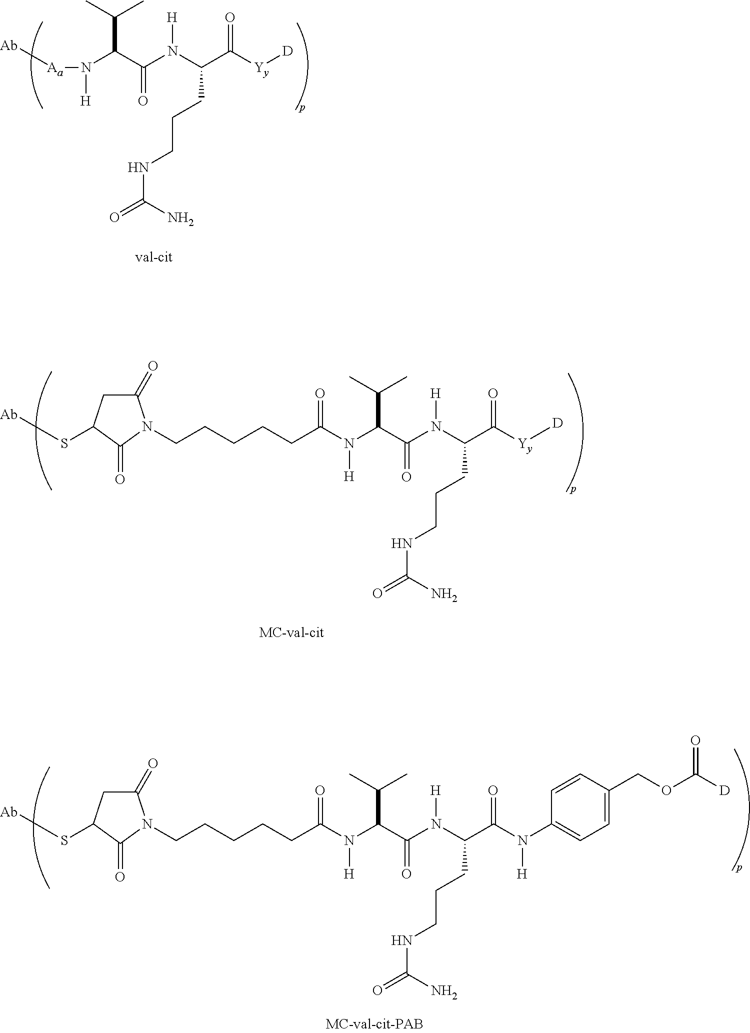

Other exemplary embodiments of the Formula Ia antibody-drug conjugate compounds include the structures:

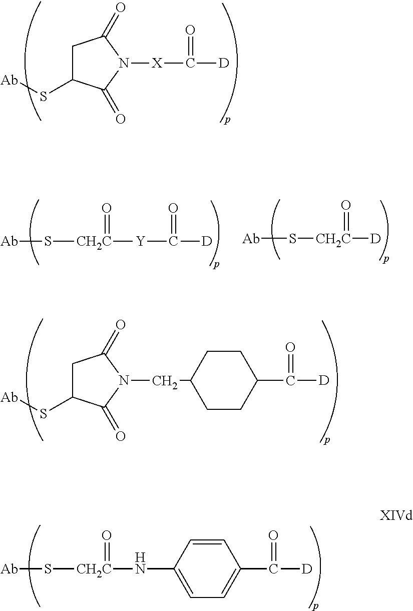

where X is:

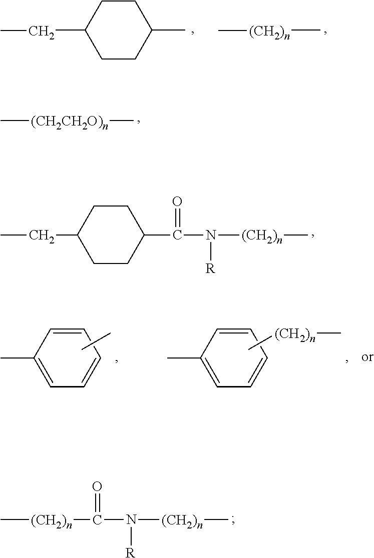

Y is:

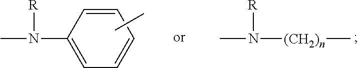

and R is independently H or C₁-C₆ alkyl; and n is 1 to 12.

In another embodiment, a Linker has a reactive functional group which has a nucleophilic group that is reactive to an electrophilic group present on an antibody. Useful electrophilic groups on an antibody include, but are not limited to, aldehyde and ketone carbonyl groups. The heteroatom of a nucleophilic group of a Linker can react with an electrophilic group on an antibody and form a covalent bond to an antibody unit. Useful nucleophilic groups on a Linker include, but are not limited to, hydrazide, oxime, amino, hydrazine, thiosemicarbazone, hydrazine carboxylate, and arylhydrazide. The electrophilic group on an antibody provides a convenient site for attachment to a Linker.

Typically, peptide-type Linkers can be prepared by forming a peptide bond between two or more amino acids and/or peptide fragments. Such peptide bonds can be prepared, for example, according to the liquid phase synthesis method (E. Schröder and K. Lübke (1965) “The Peptides”, volume 1, pp 76-136, Academic Press) which is well known in the field of peptide chemistry.

In another embodiment, the Linker may be substituted with groups that modulate solubility or reactivity. For example, a charged substituent such as sulfonate (—SO₃⁻) or ammonium, may increase water solubility of the reagent and facilitate the coupling reaction of the linker reagent with the antibody or the drug moiety, or facilitate the coupling reaction of Ab-L (antibody-linker intermediate) with D, or D-L (drug-linker intermediate) with Ab, depending on the synthetic route employed to prepare the ADC.

The compounds of the invention expressly contemplate, but are not limited to, ADC prepared with linker reagents: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and including bis-maleimide reagents: DTME, BMB, BMDB, BMH, BMOE, BM(PEG)₂, and BM(PEG)₃, Bis-maleimide reagents allow the attachment of the thiol group of a cysteine engineered antibody to a thiol-containing drug moiety, label, or linker intermediate, in a sequential or concurrent fashion. Other functional groups besides maleimide, which are reactive with a thiol group of a cysteine engineered antibody, drug moiety, label, or linker intermediate include iodoacetamide, bromoacetamide, vinyl pyridine, disulfide, pyridyl disulfide, isocyanate, and isothiocyanate.

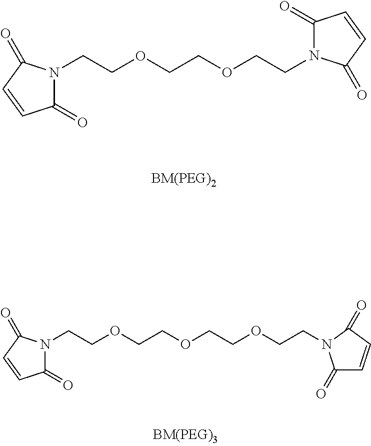 [SMILES: O=C1C=CC(=O)N1CCOCCOCCN1C(=O)C=CC1=O ; O=C1C=CC(=O)N1CCOCCOCCOCCN1C(=O)C=CC1=O]

Useful linker reagents can also be obtained via other commercial sources, such as Molecular Biosciences Inc. (Boulder, Colo.), or synthesized in accordance with procedures described in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Exemplary antibody-drug conjugate compounds of the invention include:

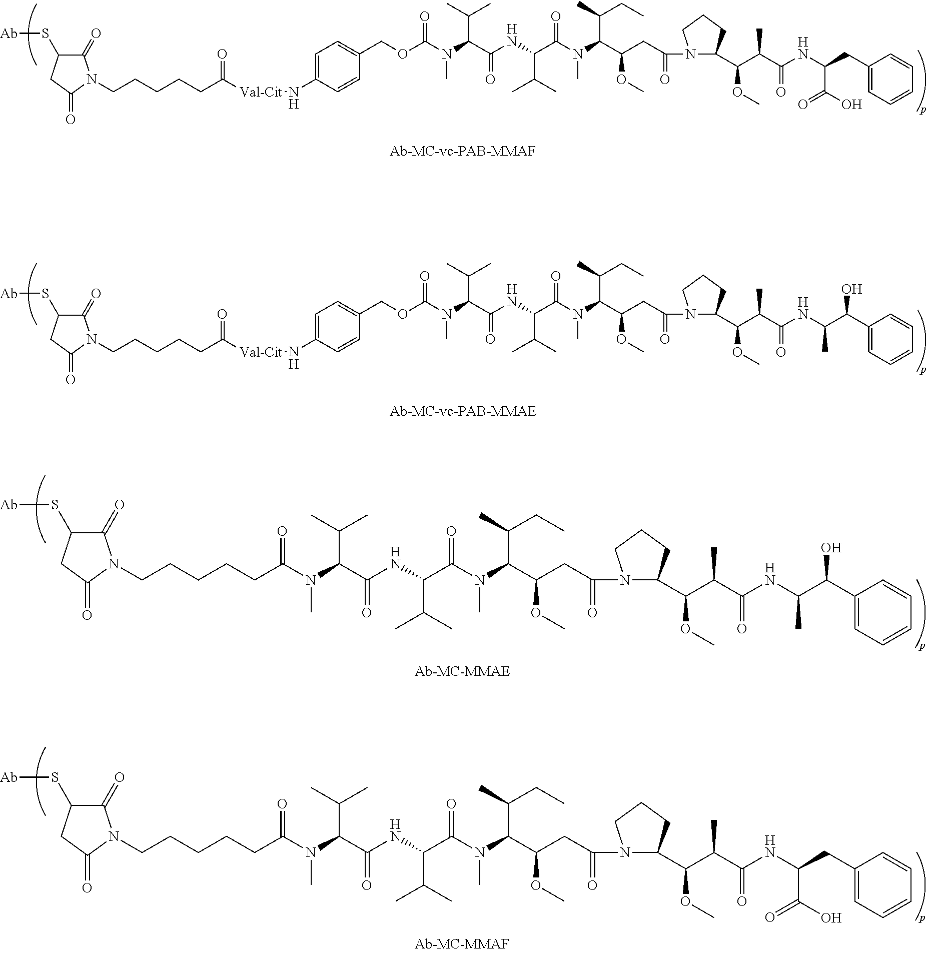

where Val is valine; Cit is citrulline; p is 1, 2, 3, or 4; and Ab is a cysteine engineered antibody.

Exemplary anti-tubulin antibody drug conjugates where maytansinoid drug moiety DM1 is linked through a BMPEO linker to a thiol group of an antibody (Ab) have the structure:

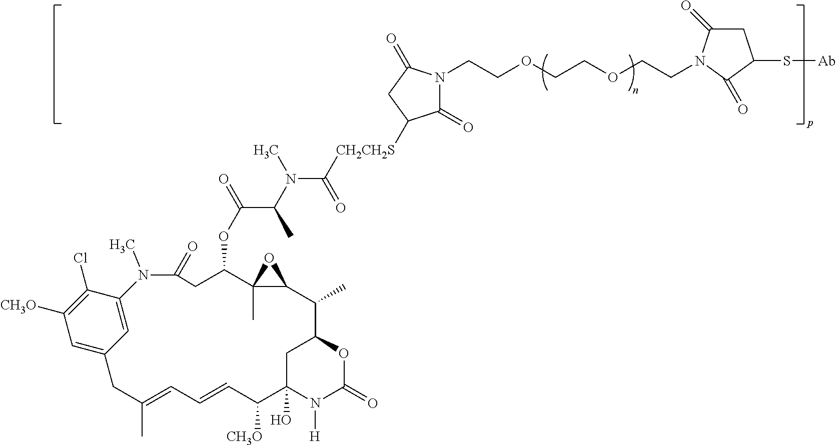

where n is 0, 1, or 2; and p is 1, 2, 3, or 4.

Other exemplary anti-tubulin antibody drug conjugates where maytansinoid drug moiety DM1 is linked through an MCC linker to a thiol group of an antibody (Ab) have the structure:

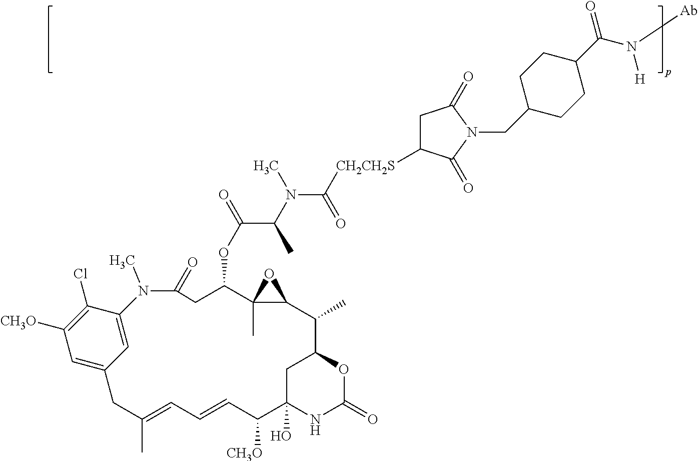

where p is 1, 2, 3, or 4.

### Anti-Tubulin Chemotherapeutic Efficacy is Regulated by Mcl-1 and FBW7

FIG. 1 shows Bcl-2 family proteins regulate cell death induced by anti-tubulin chemotherapeutic agents. (A-D) Viability of cell lines treated 48 hours with indicated agents (data are presented as the mean±SEM, n=3). BAX^{-/-}/BAK^{-/-} MEFs (a) and FDM cells (b) are resistant to antimitotic-induced cell death. (c) Genetic deletion of MCL-1 and BCL-X enhances sensitivity to paclitaxel (TAXOL®). (d) Genetic deletion of MCL-1 but not BCL-X enhances sensitivity to vincristine. FIG. 1E shows assessment of Bcl-2 family protein levels in mitotic arrest. The mitotic time course indicates when synchronized cells were collected relative to the onset of mitotic arrest: i.e. −2 is 2 hours prior to mitosis (M) and +3 is 3 hours after cells entered mitosis. CDC27 and tubulin are indicators of mitotic arrest and equal loading, respectively. cdc27-P=phosphorylated cdc27.

The sensitivity of MEFs lacking individual Bcl-2 family members to killing by paclitaxel or vincristine, two mechanistically distinct anti-tubulin chemotherapeutics, was determined. BCL-X^{-/-} cells were more sensitive than wild-type cells to paclitaxel, whereas MCL-1^{-/-} cells showed enhanced sensitivity to both paclitaxel and vincristine (FIG. 1C,D). Since ratios of pro-survival and pro-apoptotic Bcl-2 family proteins dictate cell fate their levels were monitored during mitotic arrest, as indicated by cdc27 phosphorylation (King, R. W. et al. (1995) Cell 81:279-288). Mcl-1 declined markedly in synchronized cells released into nocodazole or paclitaxel (FIGS. 1E, S4). The decrease in Noxa likely is an indirect consequence of Mcl-1-regulated stability. Mcl-1 also declined in unsynchronized cells arrested in mitosis (FIGS. S5, S34). MCL-1 transcription was not decreased during mitotic arrest (FIG. 2A). This implicated a role for the ubiquitin/proteasome system, the primary conduit for regulated protein degradation in eukaryotic cells (Finley, D. (2009) Annual review of biochemistry 78:477-513), in Mcl-1 reduction. Indeed, the proteasome inhibitor MG132 blocked Mcl-1 degradation (FIGS. 2B, S6) and endogenous Mcl-1 was ubiquitinated during mitotic arrest (FIG. S7).

FIGS. 2(A-F) show SCF^{FBW7} targets Mcl-1 for proteasomal degradation in mitotic arrest. Human carcinoma cell lines were synchronized and collected throughout the mitotic time course as in FIG. 1A (numbers indicate molecular mass in kDa). 2A: During mitotic arrest, MCL1 (Mcl-1) mRNA levels are not significantly decreased relative to MCL1 protein, as determined by WB. MC1.1 expression was monitored by real-time PCR, and the percentage mRNA is indicated relative to the 24-h time point. 2B: MG132 stabilizes MCL1 degradation during mitotic arrest in HeLa cells. 2C: RNAi oligonucleotides targeting FBW7, but not control scrambled RNAi or RNAi oligonucleotides targeting BTRC (which encodes beta-TRCP), attenuate MCL1 degradation during mitotic arrest in HCT 116 cells. 2D: MCL1 degradation is attenuated in FBW7−/− HCT 116 cells during mitotic arrest. Complementation with the alpha-isoform or beta-isoform of FBW7 restores MCL1 degradation. 2E: FBW7 recruits MCL1 to the SCF ubiquitin ligase complex core, the components of which are CUL1, SKP1 and ROC1, in HCT 116 cells in mitotic arrest. IP, immunoprecipitation, 2F: Left, reconstitution of the SCFBW7 ubiquitin ligase complex promotes Mcl-1 ubiquitylation in vitro. Ubiquitinylation reactions containing the indicated components were reacted in vitro with biotinylated ubiquitin. Reacted components were denatured, and Flag-MCL1 was immunoprecipitated (IP) and blotted (WB) for biotin to reveal in vitro ubiquitylated MCL1 (MCL1-Ub). Myc-tagged F-box proteins (including F-box-deleted FBW7 (FBW7-ΔFBox)) Flag-MCL1 and HA-tagged CUL1 variants were also immunoprecipitated and analysed as indicated by WB analysis to reveal the respective input levels. Wedges indicate an increasing amount of the indicated reaction component, Right, endogenous ROC1 does not associate with dominant-negative (DN) HA-tagged CUL1. E1, ubiquitin-activating enzyme; UBCH5A, E2 ubiquitin-conjugating enzyme,

Mcl-1 contains potential degron motifs for association with the F-box proteins beta TrCP (FBXW1, FWD1, Frescas, D. and Pagano, M. (2008) Nature reviews 8:438-449) and FBW7 (FBXW7, AGO, CDC4, SEL10, Welcker, M. & Clurman, B. E. (2008) Nature reviews 8:83-93) (FIG. S8). F-box proteins are substrate receptors for SKP1/CUL1/F-box (SCF)-type ubiquitin ligase complexes that mediate degradative polyubiquitination (Deshaies, R. J. & Joazeiro, C. A. (2009) Annual review of biochemistry 78:399-434). Consistent with a role for CUL1-based ligases in Mcl-1 turnover, ectopic expression of dominant negative CUL1 blocked Mcl-1 degradation during mitotic arrest (FIG. S9). These data suggest that the Mcl-1 ubiquitin ligase MULE (Zhong, Q., et al (2005) Cell 121:1085-1095) has a lesser role in regulating Mcl-1 turnover in mitotic arrest, a notion corroborated by MULE RNAi in paclitaxel-treated cells (FIGS. S10(A-C)). FBW7 but not beta TrCP RNAi attenuated Mcl-1 degradation in tumor cells (FIGS. 2C, S11,12) and untransformed cells (FIG. S13A,B). Mcl-1 degradation (FIG. 2D) and turnover (FIG. S14) was protracted in FBW7-null cells relative to parental cells and complementation with FBW7 isoforms restored Mcl-1 degradation (FIG. 2D, S15). Endogenous Mcl-1 was recruited to cellular SCF complex subunits in FBW7-wild-type but not FBW7-null cells in mitotic arrest (FIG. 2E). Recombinant Mcl-1 was ubiquitinated in vitro by reconstituted SCF^{FBW7} only when the complete ligase complex was assembled (FIG. 2F). Collectively, these results demonstrate that SCF^{FBW7} promotes Mcl-1 degradation in mitotic arrest.

Because substrate phosphorylation promotes recruitment to FBW7 (Welcker, M. and Clurman, B. E. (2008) Nature reviews 8:83-93), the phosphorylation status of candidate FBW7 degrons on Mcl-1 was evaluated in cells arrested in mitosis (FIG. 3A). Mass spectrometry revealed phosphorylation of residues S64, S121, S159, and T163 (FIGS. 3A, S16(A-D)). Myc-tagged Mcl-1 was efficiently recruited to FLAG-FBW7 in mitotic arrest (FIG. S17) and Mcl-1 residues 1-170 directed FBW7 binding (FIG. S18), thus mutant Mcl-1 constructs were tested to identify the degrons that confer FBW7 association (FIG. 3A). Mcl-1 mutants S121A/E125A and S159A/T163A bound FBW7 less efficiently (FIG. 3B) and their degradation was attenuated in mitotic arrest (FIG. 3c). Assessment of the relative affinities of the Mcl-1 degrons for FBW7 revealed that S121/E125 binds tighter (FIG. 3D,E). Thus, similar to other FBW7 substrates such as cyclin E (Welcker, M. and Clurman, B. E. (2008) Nature reviews 8:83-93), Mcl-1 contains high- and low-affinity FBW7 degrons, both of which are required for efficient recruitment to (FIG. 3b) and subsequent degradation by (FIG. 3C) SCF^{FBW7} in the context of full length Mcl-1.

FIGS. 3(A-G) show identification of MCL1 degron motifs and protein kinases that direct recruitment to FBW7 during mitotic arrest. 3A: The FBW7 degron consensus sequence (top, with potential phosphorylation sites or phosphomimic residues), corresponding MCL1 residues (centre) and confirmed phosphorylation sites (P) during mitosis are indicated for three MCL1-derived peptide sequences. Phosphorylation at 5159 rather than 5162 was confirmed by co-elution with a synthetic peptide (see Supplementary FIG. 16). h, hydrophobic amino acid; X, any amino acid. The MCL1 (Mcl-1) phospho-mutant nomenclature used is indicated. 3B: Association of Flag-FBW7 with Myc-MCL1 mutants S121A/E125A, S159A/T163A, and 4A is attenuated in mitotic arrest. The indicated constructs were expressed in HeLa cells that were synchronized, released into Taxol (paclitaxel)), and processed as indicated. 3C: MCL1 phospho-mutants S121A/E125A, S159A/T163A and 4A have attenuated degradation during mitotic arrest. HCT116 cells were synchronized and collected throughout the mitotic time course as in FIG. 1A. 3D: Schematic representation of MCL1- or cyclin-E-derived peptides and their calculated dissociation constants (Kd), averaged from duplicate experiments (mean6s.d.), for FBW7 binding as determined by ELISA. 3E: The MCL1-derived peptide containing the phosphorylated S121/E125 degron (MCL1 S121-P) preferentially binds to FBW7 in vitro. Graphical representation of the fraction of FBW7-bound cyclin E or MCL1 peptides as a function of peptide concentration is shown. DMSO, dimethyl sulphoxide 3F: Pharmacological inhibition of INK, p38 or CDK1 (with inhibitor (and targeted kinase) indicated, top) attenuates recruitment of Myc-MCL1 to Flag-FBW7 during mitotic arrest. The indicated constructs were expressed in HeLa cells with or without CDC20 RNAi oligonucleotides or control scrambled RNAi oligonucleotides, and cells were then synchronized and released into Taxol. When cells entered mitotic arrest, the indicated agents were added for 1 h followed by a 3-h incubation with 25 mM MG132 before collection and processing as indicated (see FIG. S25). 3G, in vitro phosphorylation of recombinant MCL1 drives FBW7 binding. Full-length MCL1 was subjected to in vitro phosphorylation with the indicated kinases and subsequently incubated with recombinant Flag-FBW7. Anti-Flag immunoprecipitates were resolved by SDS-PAGE and probed with antibodies specific for the indicated proteins.

Kinase(s) that direct Mcl-1 recruitment to FBW7, have Mcl-1 degron consensus sites and demonstrate activity in mitotic arrest include cdk1, CKII, ERK, GSK3-b, JNK, and p38 (FIGS. S19, S24c). Studies with kinase inhibitors (FIGS. S20A, S21, S22(A-B), S24(A-B)) or RNAi (FIGS. S20B, S23(A-C), S24(A-C)) indicated that JNK, p38, CKII, and cdk1 activities regulate Mcl-1 degradation in mitotic arrest. Since cdk1 inhibition drives cells out of mitosis (Potapova, T. A. et al. (2006) Nature 440:954-958) (FIGS. S21, S22(A-B)) non-degradable cyclin B1 or cdc20 RNAi was expressed to maintain cells in mitotic arrest (Huang, et al. (2009) Cancer cell 16:347-358) (FIGS. 24(A-B)) Inhibition of JNK, p38, or cdk1 also attenuated Mcl-1 recruitment to FBW7 (FIGS. 3F, S25, S26). JNK, p38, and CKII, but not cdk1, directly phosphorylated Mcl-1 degrons (Tables 1a-1c). JNK and p38 directly promote Mcl-1/FBW7 binding whereas the effect of cdk1 is negligible (FIG. 3g), suggesting that cdk1 indirectly enhances Mcl-1 phosphorylation to promote FBW7 binding in the cellular context. Indeed, cdk1 phosphorylates T92 (Table 1d), a residue that is phosphorylated (FIG. S16E) and regulates Mcl-1 turnover (FIG. S27A) in mitotic arrest. As the phosphatase inhibitor okadaic acid (OA) and paclitaxel similarly regulate Mcl-1 phosphorylation (Domina, et al (2004) Oncogene 23:5301-5315), cdk1-directed T92 phosphorylation was found to block association of the OA-sensitive phosphatase PP2A with Mcl-1 in mitotic arrest. PP2A more readily dissociated from wild-type Mcl-1 relative to the T92A mutant concomitant with increasing cdk1 activity (FIG. S27B). Mcl-1-associated PP2A protein and phosphatase activity are low in mitotic arrest when cdk1 activity is high but are restored after mitotic exit when cdk1 is inactivated (FIG. S27C). Thus phosphorylation of Mcl-1 degron residues by JNK, p38, and CKII in mitotic arrest is likely initially opposed by phosphatases such as PP2A. Maximal activation of cdk1 in prolonged mitotic arrest promotes T92 phosphorylation and PP2A dissociation, permitting sufficient phosphorylation of Mcl-1 degron residues to drive FBW7-mediated degradation (FIG. S1). These effects are revealed when microtubule-targeted agents are washed out of cells in mitotic arrest: JNK, p38 and cdk1 activities decline and Mcl-1 levels are restored (FIG. S28). Sufficient loss of Mcl-1 activates Bak and Bax (FIG. S29) to promote apoptosis.

FBW7 mutations identified in patient-derived cell lines disrupted association with Mcl-1 in mitotic arrest (FIG. S30); thus, failure of inactivated FBW7 to promote Mcl-1 degradation could confer resistance to anti-tubulin chemotherapeutics. Indeed, FBW7-null cell lines displayed attenuated Mcl-1 degradation and were more resistant to paclitaxel- or vincristine-induced cell death relative to wild-type cells (FIG. S31, S32). Bcl-x_{L} remained stable regardless of FBW7 status (FIG. S31). Similar trends were seen in patient-derived ovarian (FIG. 4A) and colon (FIG. S33) cancer cell lines harboring naturally-occurring FBW7 mutations. Although responses to antimitotic agents are heterogeneous within cell populations (Gascoigne, K. E. and Taylor, S. S. (2008) Cancer cell 14:111-122) mitotic arrest was robustly activated in asynchronous ovarian cancer cell lines (FIG. S34). Moreover, Mcl-1 degradation profiles were similar in synchronized and asynchronous cells: Mcl-1 was efficiently degraded in FBW7-wild-type cells, yet Mcl-1 persisted in FBW7-mutant SKOV3 cells and in TOV21G cells that undergo only transient mitotic arrest (FIGS. 4A, S34). Thus the survival of cells arrested in mitosis is dictated by Mcl-1, that is in turn regulated by FBW7.

FIGS. 4(A-E) show FBW7 inactivation and increased MCL1 levels promote anti-tubulin agent resistance and tumorigenesis in human cancers, 4A: FBW7-WT ovarian cancer cell lines that undergo mitotic arrest are sensitive to Taxol (left) and rapidly degrade MCL-1 relative to FBW7-mutant and Taxol-resistant cells (right). FBW7 status is specified in parentheses. 4B: Sensitivity to vincristine-induced cell death is restored in FBW7−/− cells on MCL1 ablation. WT or FBW7−/− HCT 116 cells were transduced with the indicated doxycycline-inducible shRNA constructs, cultured in the presence of doxycycline, and treated with various concentrations of vincristine for 48 h before cell viability assessment. shLacZ, control shRINA. Data are presented as mean±s.e.m.; n=53. 4C: MCL1 expression modulates polyploidy in FBW7-deficient HCT 116 cells. WT or FBW7−/− HCT 116 cells were transduced with the indicated doxycycline-inducible snRNA constructs, cultured in the presence of doxycycline, synchronized and released into vincristine, They were then collected at 5 h (15h) or 10 h (110 h) after mitotic arrest and fixed, stained with propidium iodide and analysed by FACS (x axis, fluorescence units; y axis, number of cells). M1, percentage of cells with >2N DNA content. 4D: MCL1 expression increases mitotic slippage and attenuates apoptosis in FBW7-deficient cells. WT or FBW7−/− HCT 116 cells were transduced with the indicated doxycycline-inducible shRNA constructs, cultured in the presence of doxycycline, transduced with an H2B-GFP-expressing baculovirus, synchronized, treated with the indicated anti-tubulin agents and imaged live. Three images were acquired every 10 min for 43 h, and 50 cells were analyzed for each condition. *, P,0.05; **, P,0.001 (one-tailed Fisher's exact test). 4E: MCL1 levels are elevated in non-small-cell lung cancer (NSCLC) samples with mutant FBW7 or low FBW7 copy number relative to FBW7-WT tumours and normal lung samples (see also Supplementary Table 2). NSCLC FBW7-mutant samples 3 and 5 also have low FBW7 copy number.

The FBW7 R505L mutant protein was expressed in FBW7-wild-type TOV112D-X1 cells to mimic cells harboring one mutated FBW7 allele (Welcker, M. and Clurman, B. E. (2008) Nature reviews 8:83-93) and to assess the in vivo effects. Tumors expressing mutant FBW7 were more resistant to paclitaxel (FIG. S35A) and had elevated Mcl-1 relative to FBW7-wild-type parental tumors (FIGS. S35(B-C)). Bcl-X_{L} was unaffected by FBW7 status (FIGS. S35(B,D)). Reducing Mcl-1 protein in FBW7-null cells restored their sensitivity to paclitaxel- and vincristine-induced death (FIGS. 4B, S36), demonstrating that Mcl-1 is a critical pro-survival factor responsible for resistance to antimitotic agents in FBW7-deficient cells.

Previous studies have shown that blocking apoptosis in mitotic arrest permits cells to exit mitosis and evade cell death (Gascoigne, K. E. and Taylor, S. S. (2008) Cancer cell 14:111-122), and that FBW7 null cells more frequently exit mitosis and undergo endoreduplication to render cells polyploidy (Finkin, S., et al (2008) Oncogene 27:4411-4421). The results here establish Mcl-1 as an FBW7 substrate and therefore suggests a molecular link to explain antimitotic resistance and chemotherapy-induced polyploidy. Indeed, FBW7-null cells exit paclitaxel- or vincristine-induced mitotic arrest more readily (FIGS. 4d, S37, S38) and display more pronounced polyploidy (FIG. 4C) than FBW7-wild-type cells. Decreasing Mcl-1 protein levels in the FBW7-null cells blocked premature mitotic slippage (FIGS. 4D, S37, S38), reduced chemotherapeutic-induced polyploidy (FIG. 4C) and enhanced paclitaxel- or vincristine-induced apoptosis compared with FBW7-null cells treated with control shRNA (FIG. 4D). Thus Mcl-1 promotes resistance to antimitotic chemotherapeutics and facilitates genomic instability when FBW7 is inactivated.

The hostile tumor microenvironment, like chemotherapeutic insults, exerts selective pressures on malignant cells; therefore tumor cells harboring alterations in FBW7 and Mcl-1 should be selected for and enriched in primary patient tumor samples. To this end, copy number analysis of FBW7 and MCL-1 was performed in ovarian tumor samples (FIG. S39). The co-occurrence of MCL-1 gain and FBW7 loss was more frequent than expected, consistent with selection for both genetic alterations (FIG. S39). Data from NSCLC samples showed similar trends but was not statistically significant due to insufficient sample size (not shown). Immunoblotting of patient samples revealed that most FBW7-inactivated tumors had elevated Mcl-1 protein levels relative to FBW7-wild-type tumors and normal lung samples (FIGS. 4E, Supplementary Table 2). In contrast, Bcl-X_{L} was not correlated with FBW7 status (FIG. 4E). Thus functional FBW7 is required to down-regulate Mcl-1 in primary patient samples, a particularly significant finding given that antimitotic agents are therapeutic mainstays for NSCLC and ovarian cancers.

The signaling pathways that activate cell death induced by anti-tubulin chemotherapeutics are of interest. The surprising and unexpected results here provide genetic evidence that both MCL-1 and BCL-X are regulators of this therapeutic response. Whereas Bcl-X_{L} is functionally inactivated by phosphorylation (Terrano, D. T. et al (2010) Molecular and cellular biology 30:640-656) and is unaffected by FBW7 status, Mcl-1 inactivation is orchestrated by the concerted activities of phosphatases, stress-activated and mitotic kinases, and the SCF^{FBW7} ubiquitin ligase. As such, a unique molecular mechanism for Mcl-1 regulation and initiation of apoptosis in mitotic arrest is defined (FIG. S1). By identifying SCF^{FBW7} as a critical ubiquitin ligase that directs Mcl-1 degradation in mitotic arrest, a mechanism for resistance to anti-tubulin chemotherapeutics is elucidated. Analysis of patient samples suggests that drug efflux pumps (Ozalp, S. S., et al (2002) European journal of gynaecological oncology 23:337-340) or tubulin alterations (Mesquita, B. et al. (2005) BMC cancer 5:101) do not always account for antimitotic resistance, thus evasion of apoptosis due to inappropriately elevated Mcl-1 is likely a critical strategy. Increased Mcl-1 in FBW7-deficient cells promotes mitotic slippage, endoreduplication, and subsequent polyploidy in response to paclitaxel and vincristine. The role of Mcl-1 in FBW7-deficient cells therefore extends beyond simple apoptosis inhibition; facilitating genomic aberrations and fueling the transformed state.

Synthetic dolastatin analogs, auristatins such as MMAE, are anti-tubulin chemotherapeutic agents with activity as single agents (FIG. 5) and as drug moieties conjugated to antibodies targeting cell-surface receptor antigens, forming antibody-drug conjugates (ADC), (FIGS. 6-13) in promoting mitotic arrest with Mcl-1 degradation and/or Bcl-xL S62 phorphorylation in solid tumor and hematopoietic tumor cell lines. Bim-EL is also degraded, but Bim-L and Bim-S are less affected. Thus, anti-tubulin antibody-drug conjugate compounds have the surprising and unexpected effects of regulating Bcl-2 family members Mcl-1, Bim, and total and phos-S62-Bcl-xL.

FIG. 5 shows MMAE, a synthetic, anti-tubulin agent, promotes mitotic arrest and subsequent Mcl-1 degradation in Granta-519, HCT-116 and HeLa cells. M=mitosis as indicated by phospho-cdc27; −4=4h prior to mitosis; +2=2h after onset of mitotic arrest.

FIG. 6A shows the anti-tubulin antibody-drug conjugate, anti-NaPi3b-MC-vc-PAB-MMAE (ADC-MMAE) promotes mitotic arrest in OVCAR3×2.1 ovarian cancer cells, relative to a negative control, (anti-gD (glycoproteins D) ADC), a non-specific binding antibody-drug conjugate.

FIG. 6B shows levels of Mcl-1, Bim, non-pBcl-xL ser62, and phospho-histone 3 in OVCAR3×2.1 ovarian cancer cells after treatment with anti-NaPi3b-MC-vc-PAB-MMAE (ADC-MMAE) relative to negative control, non-specific binding antibody-drug conjugate (anti-gD ADC)

FIG. 7A shows the anti-tubulin antibody-drug conjugate, anti-STEAP1-MC-vc-PAB-MMAE (ADC-MMAE) promotes mitotic arrest in LNCaP prostate cancer cells, relative to a negative control, (anti-gD ADC), a non-specific binding antibody-drug conjugate.

FIG. 7B shows levels of Mcl-1, Bim, non-pBcl-xL ser62, and phospho-histone 3 in LNCaP prostate cancer cells after treatment with anti-STEAP1-MC-vc-PAB-MMAE (ADC-MMAE) relative to negative control, non-specific binding antibody-drug conjugate (anti-gD ADC)

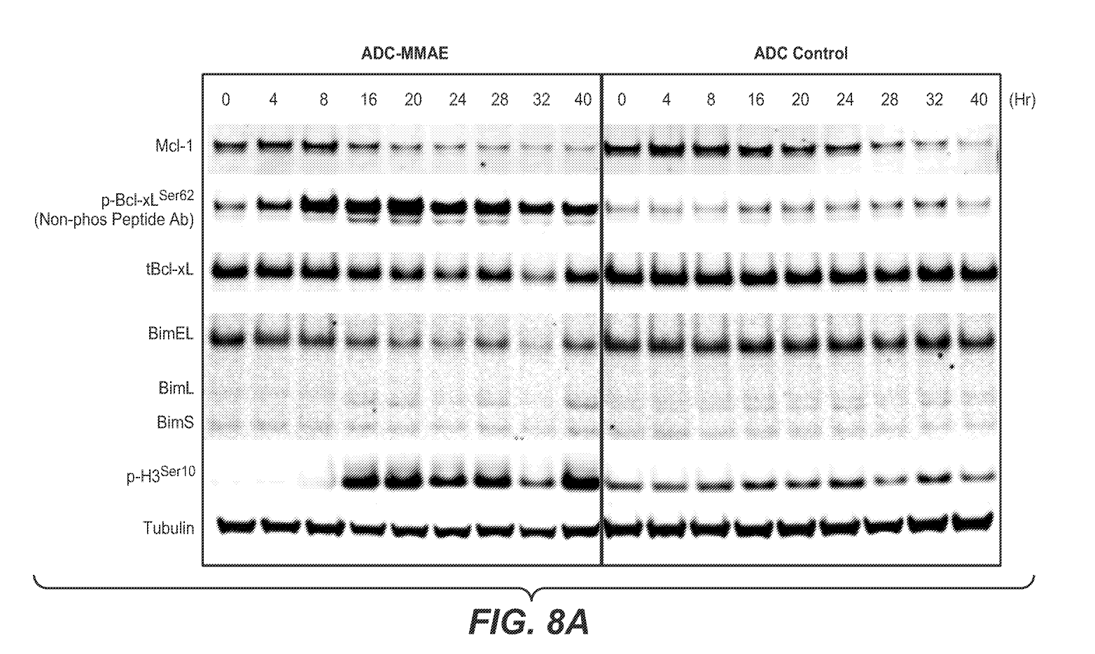

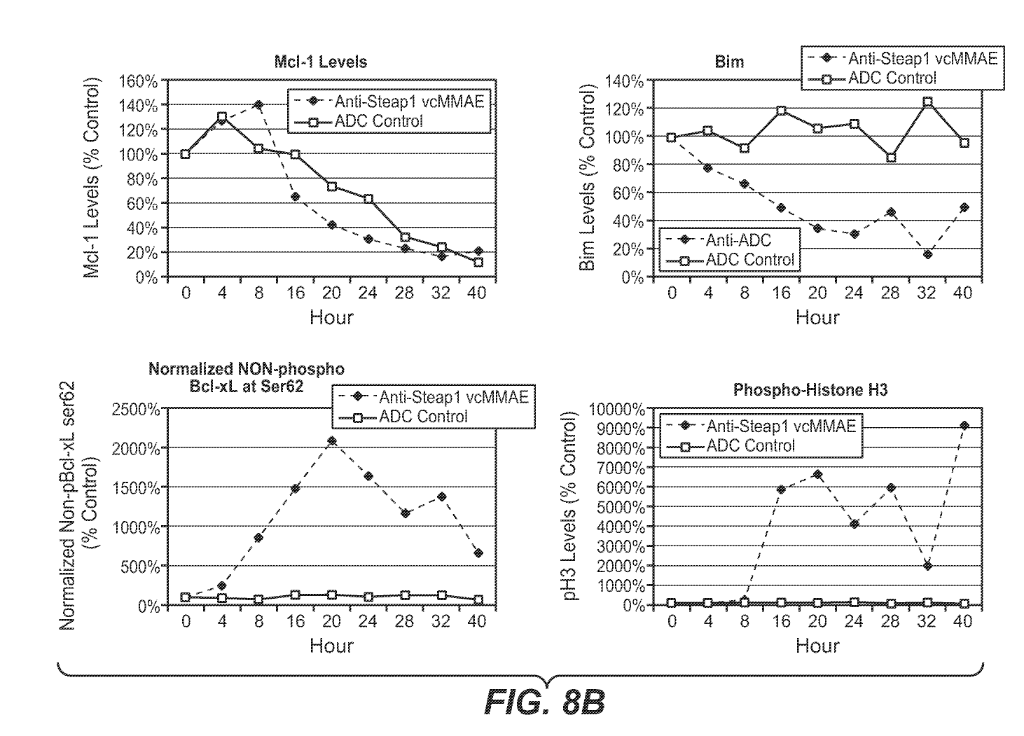

FIG. 8A shows the anti-tubulin antibody-drug conjugate, anti-STEAP1-MC-vc-PAB-MMAE (ADC-MMAE) promotes mitotic arrest in 293 cells expressing STEAP1, relative to a negative control, (anti-gD ADC), a non-specific binding antibody-drug conjugate.

FIG. 8B shows levels of Mcl-1, Bim, non-pBcl-xL ser62, and phospho-histone 3 in 293 cells expressing STEAP1 after treatment with anti-STEAP1-MC-vc-PAB-MMAE (ADC-MMAE) relative to negative control, non-specific binding antibody-drug conjugate (anti-gD ADC)

FIG. 9A shows the anti-tubulin antibody-drug conjugate, anti-ETBR-MC-vc-PAB-MMAE (ADC-MMAE) promotes mitotic arrest in UACC-257×2.2 melanoma cancer cells, relative to a negative control, (anti-gD ADC), a non-specific binding antibody-drug conjugate.

FIG. 9B shows levels of Mcl-1, Bim, non-pBcl-xL ser62, and phospho-histone 3 in UACC-257×2.2 melanoma cancer cells after treatment with anti-ETBR-MC-vc-PAB-MMAE (ADC-MMAE) relative to negative control, non-specific binding antibody-drug conjugate (anti-gD ADC)

FIG. 10A shows the anti-tubulin antibody-drug conjugate, anti-CD22-MC-vc-PAB-MMAE (ADC-MMAE) promotes mitotic arrest in Granta-519 B-cell lymphoma cancer cells, relative to a negative control, (anti-gD ADC), a non-specific binding antibody-drug conjugate.

FIG. 10B shows levels of Mcl-1, phospho-histone 3, and pBcl-xL in Granta-519 B-cell lymphoma cancer cells after treatment with anti-CD22-MC-vc-PAB-MMAE (ADC-MMAE) relative to negative control, non-specific binding antibody-drug conjugate (anti-gD ADC)

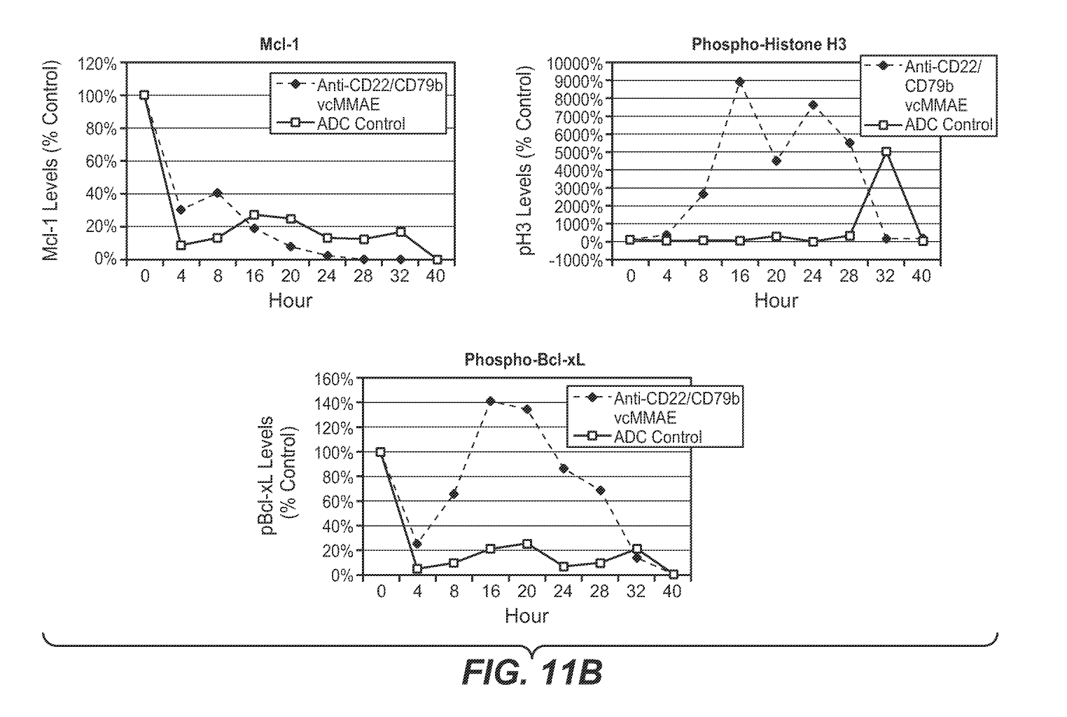

FIG. 11A shows the anti-tubulin antibody-drug conjugate, anti-CD22-MC-vc-PAB-MMAE (ADC-MMAE) promotes mitotic arrest in WSU-DLCL2 B-cell lymphoma cancer cells, relative to a negative control, (anti-gD ADC), a non-specific binding antibody-drug conjugate.

FIG. 11B shows levels of Mcl-1, phospho-histone 3, and pBcl-xL in WSU-DLCL2 B-cell lymphoma cancer cells after treatment with anti-CD22-MC-vc-PAB-MMAE (ADC-MMAE) relative to negative control, non-specific binding antibody-drug conjugate (anti-gD ADC)

FIG. 12A shows the anti-tubulin antibody-drug conjugate, anti-FcRH5-MC-vc-PAB-MMAE (ADC-MMAE) promotes mitotic arrest in EJM cells expressing FcRH5 multiple myeloma cancer cells, relative to a negative control, (anti-gD ADC), a non-specific binding antibody-drug conjugate.

FIG. 12B shows levels of Mcl-1, phospho-histone 3, and pBcl-xL in EJM cells expressing FcRH5 multiple myeloma cancer cells after treatment with anti-FcRH5-MC-vc-PAB-MMAE (ADC-MMAE) relative to negative control, non-specific binding antibody-drug conjugate (anti-gD ADC)

FIG. 13A shows the anti-tubulin antibody-drug conjugate, anti-FcRH5-MC-vc-PAB-MMAE (ADC-MMAE) promotes mitotic arrest in OPM2 cells expressing FcRH5 multiple myeloma cancer cells, relative to a negative control, (anti-gD ADC), a non-specific binding antibody-drug conjugate.

FIG. 13B shows levels of Mcl-1, phospho-histone 3, and pBcl-xL in OPM2 cells expressing FcRH5 multiple myeloma cancer cells after treatment with anti-FcRH5-MC-vc-PAB-MMAE (ADC-MMAE) relative to negative control, non-specific binding antibody-drug conjugate (anti-gD ADC)

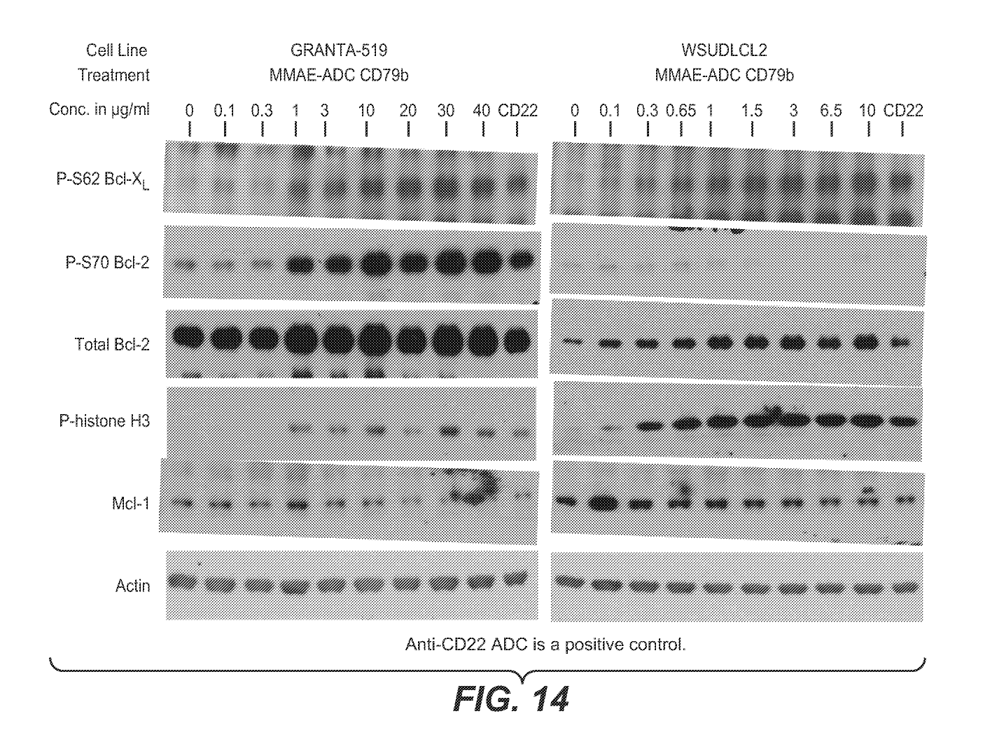

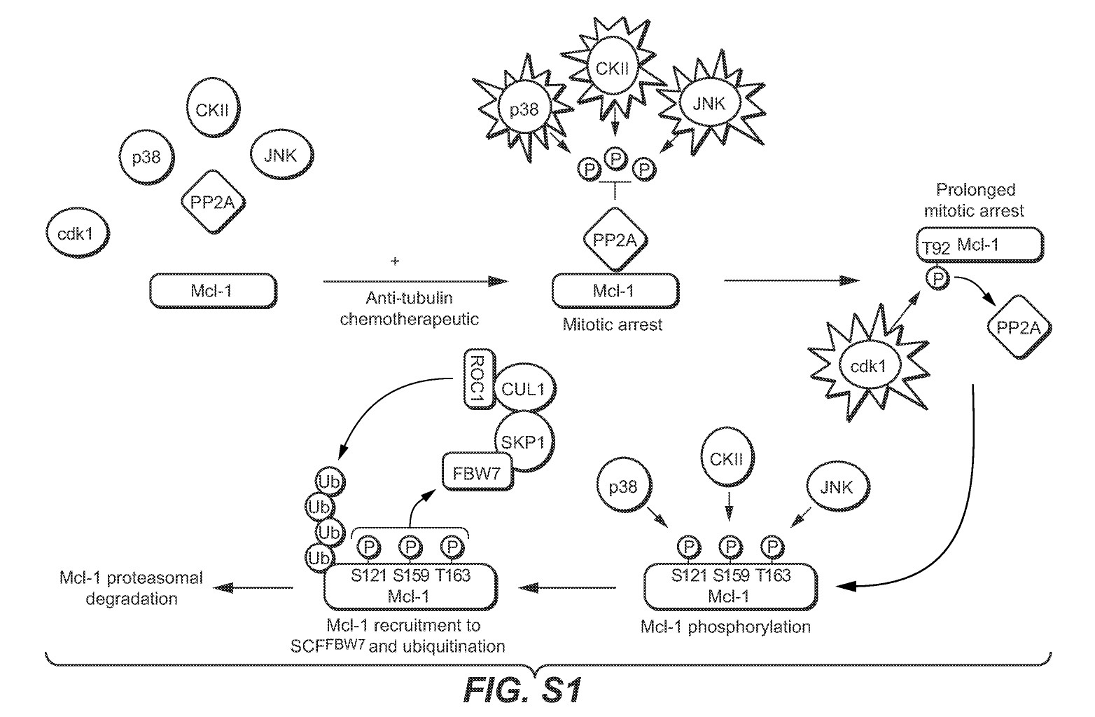

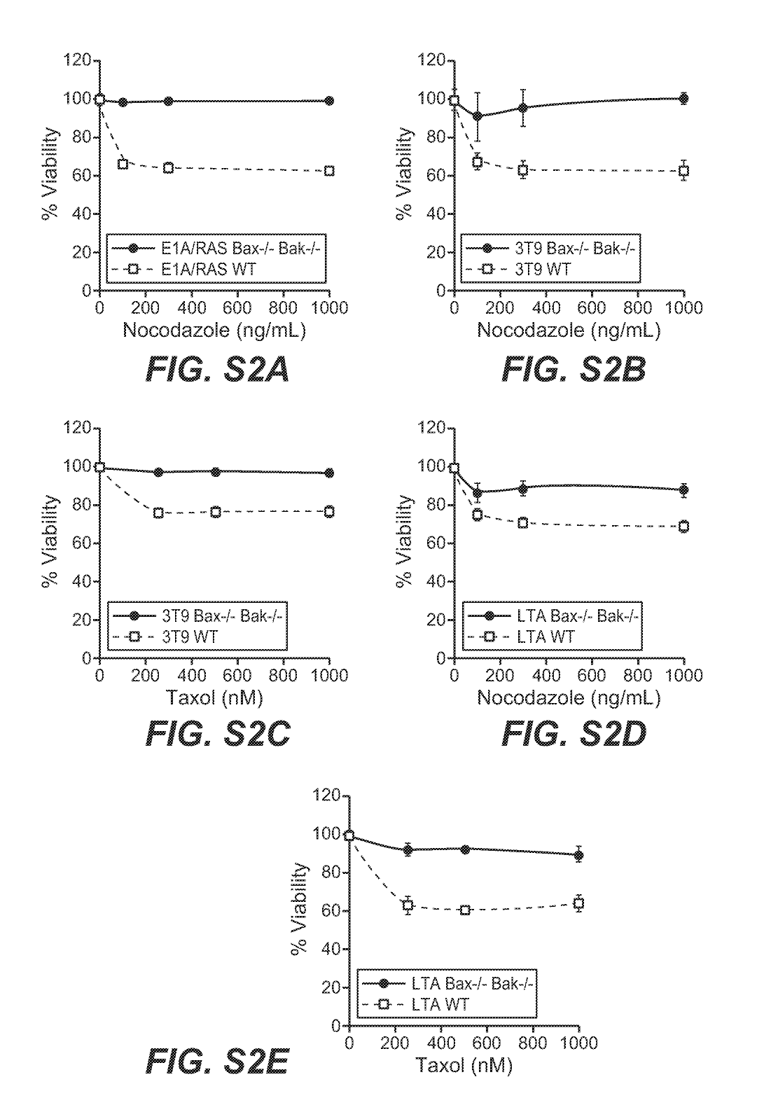

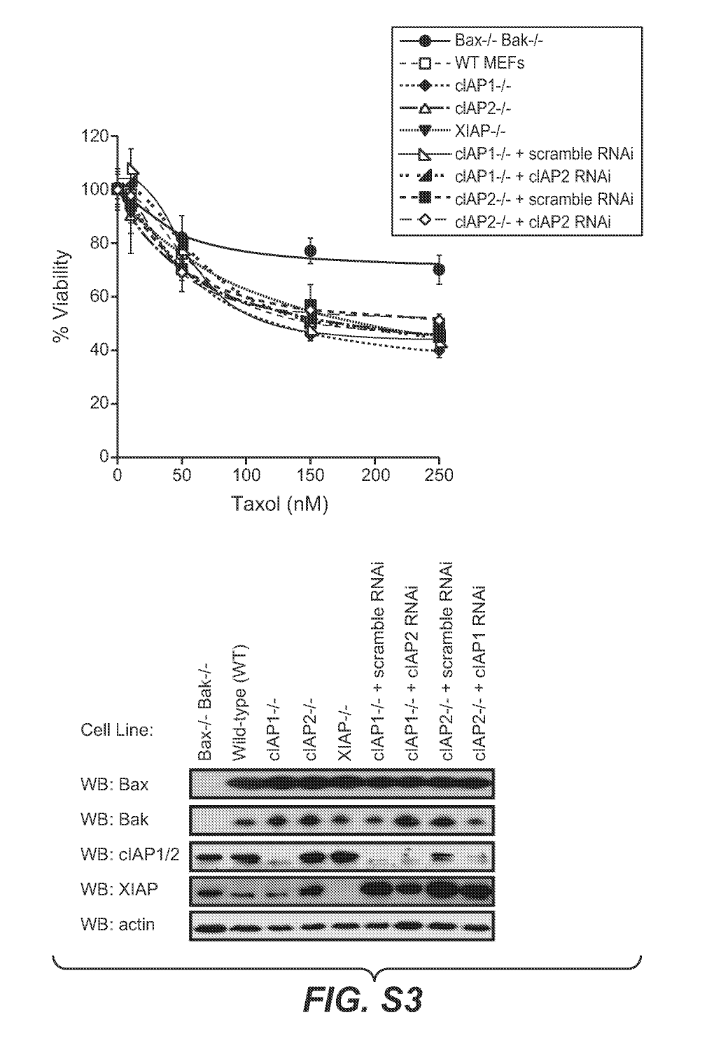

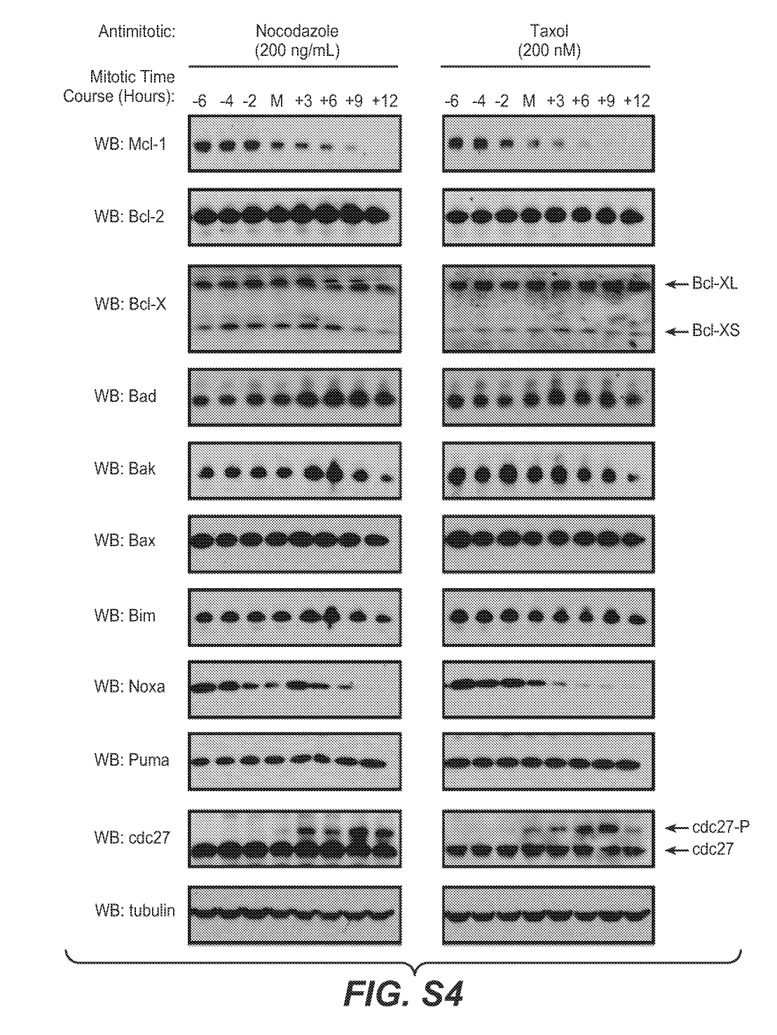

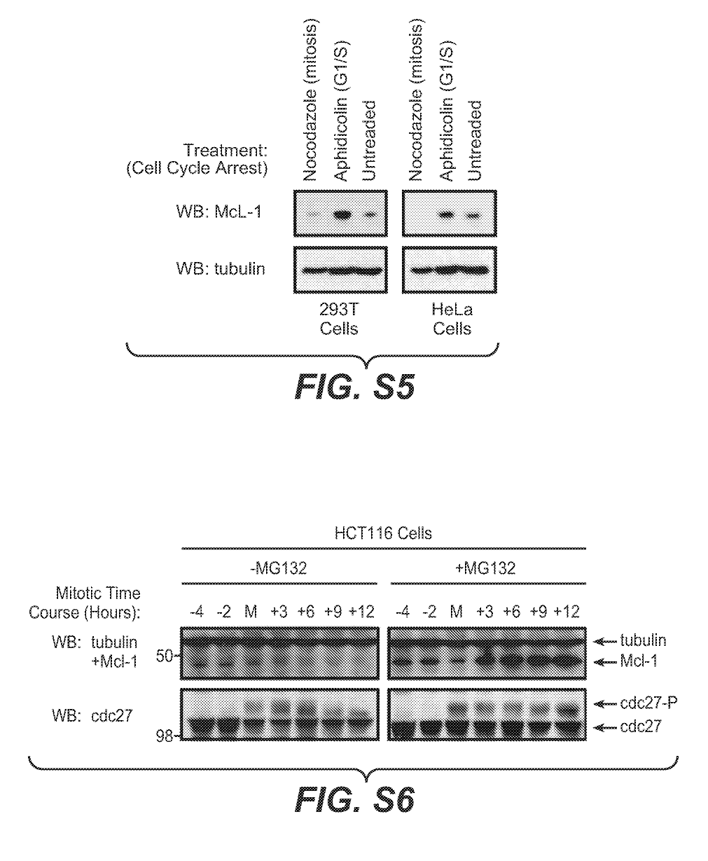

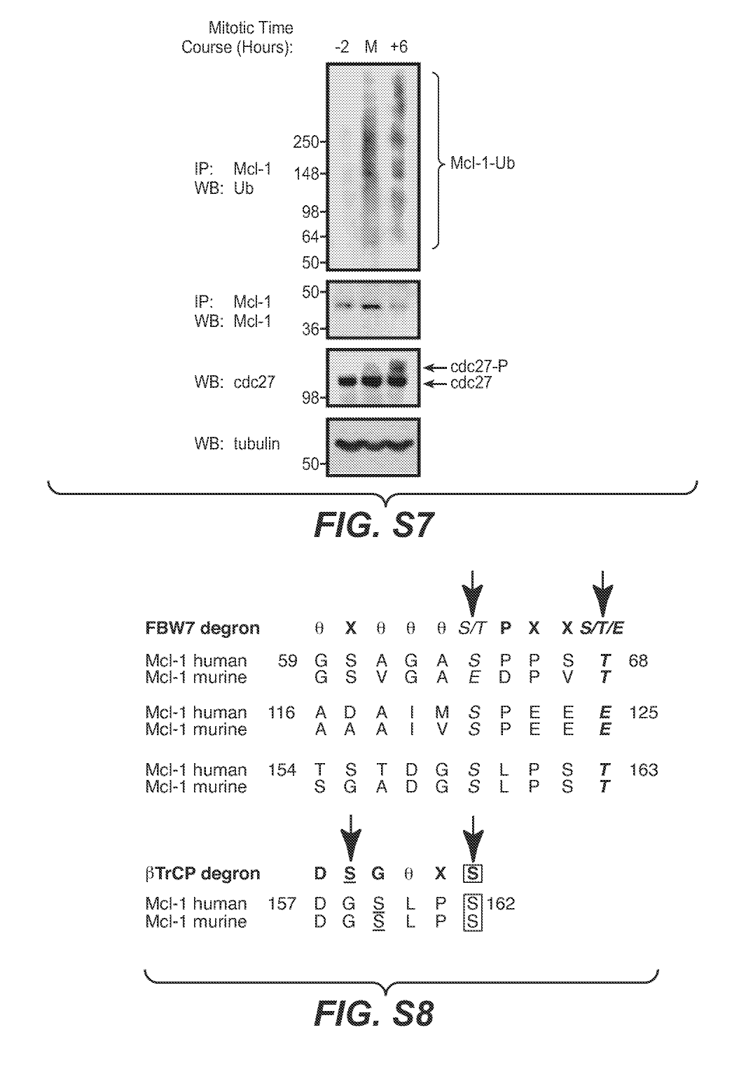

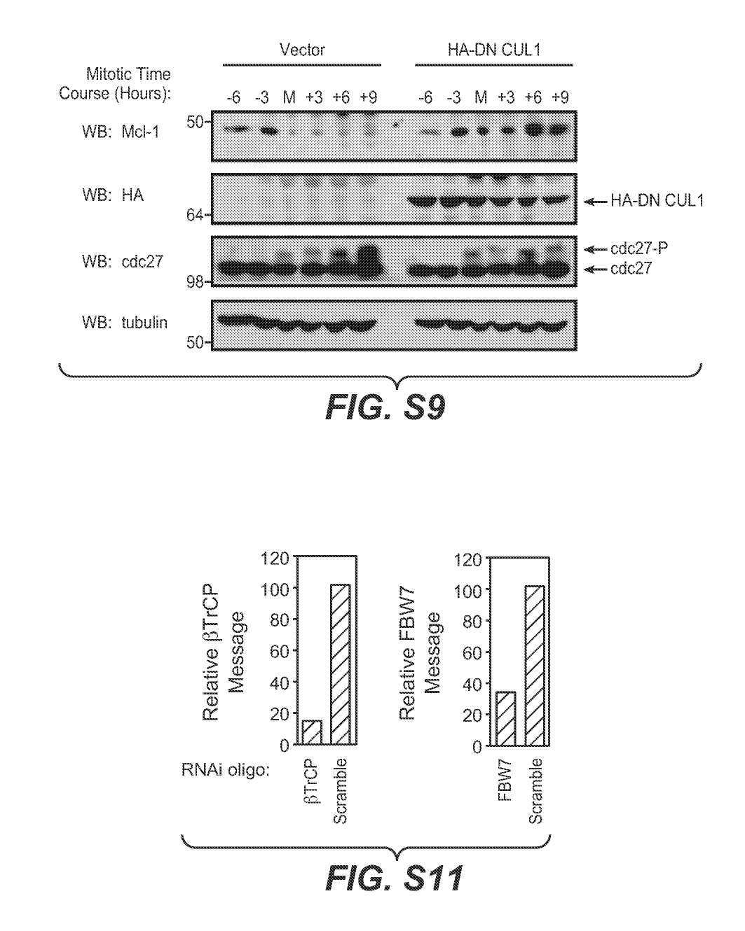

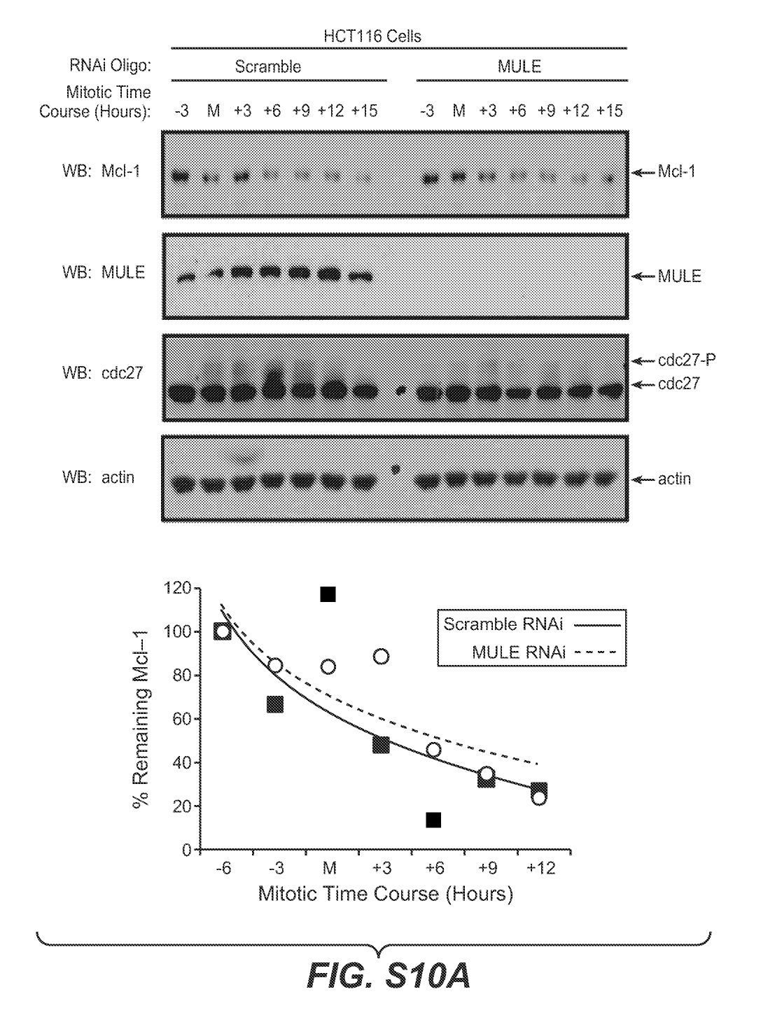

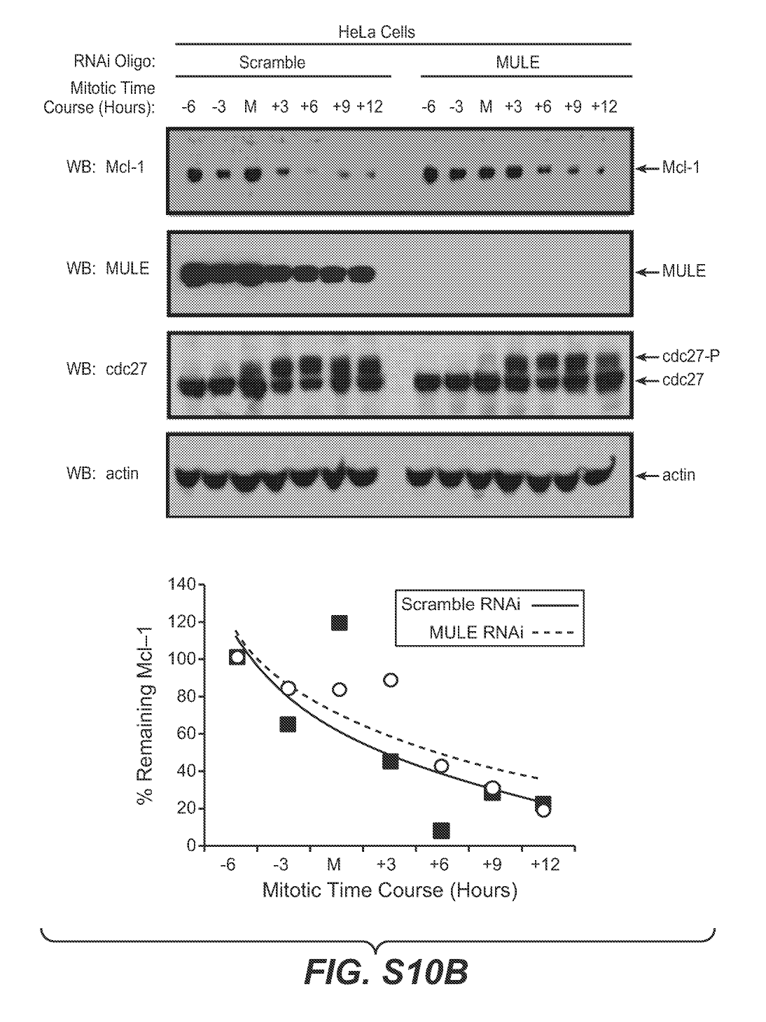

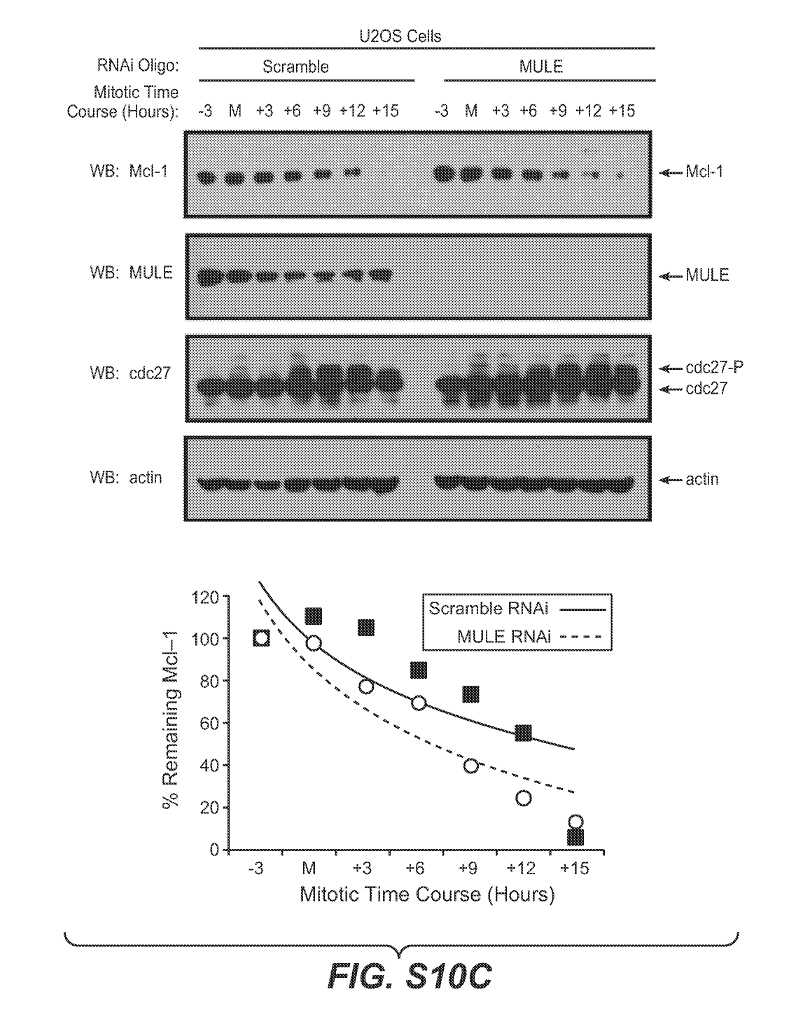

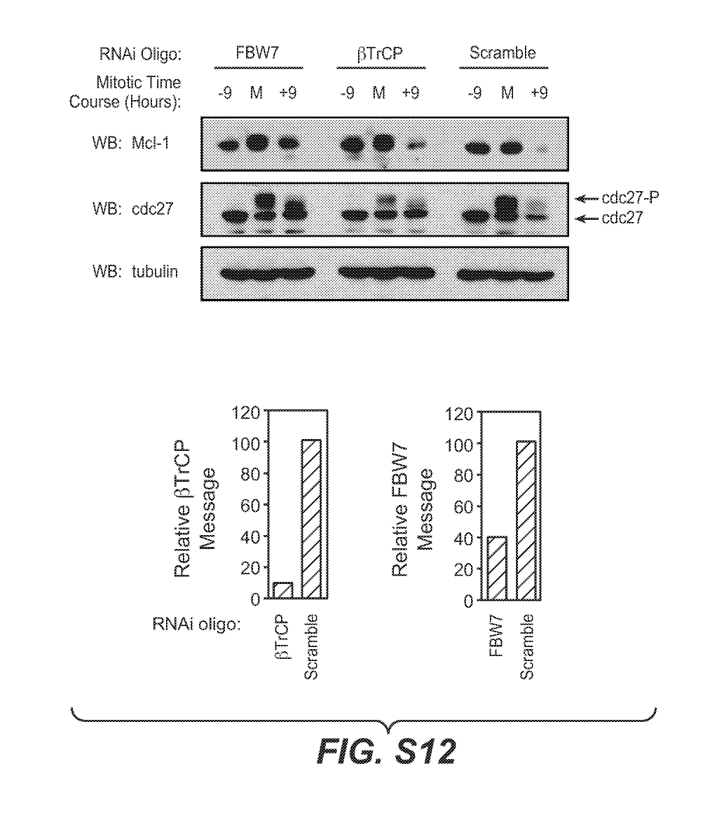

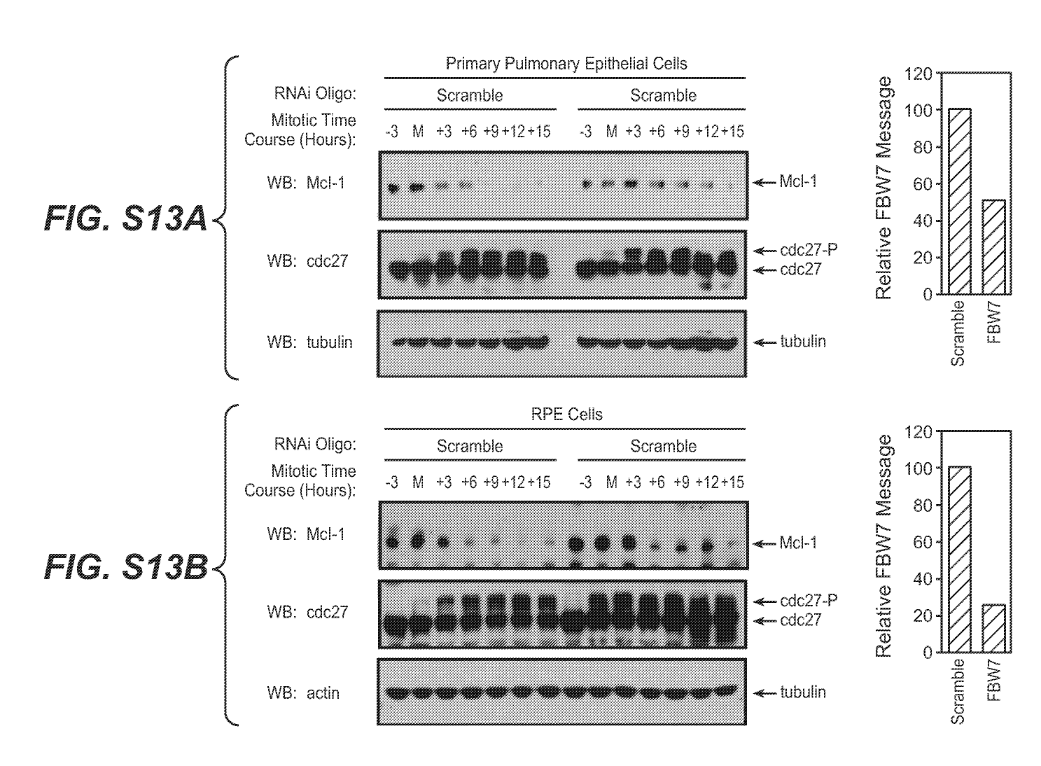

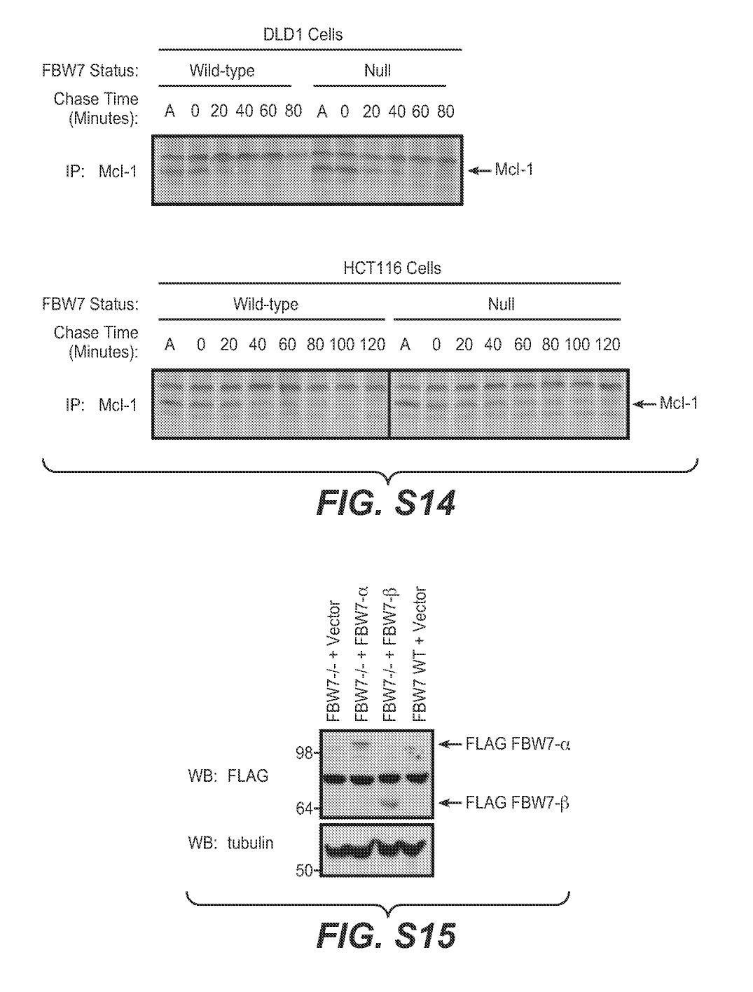

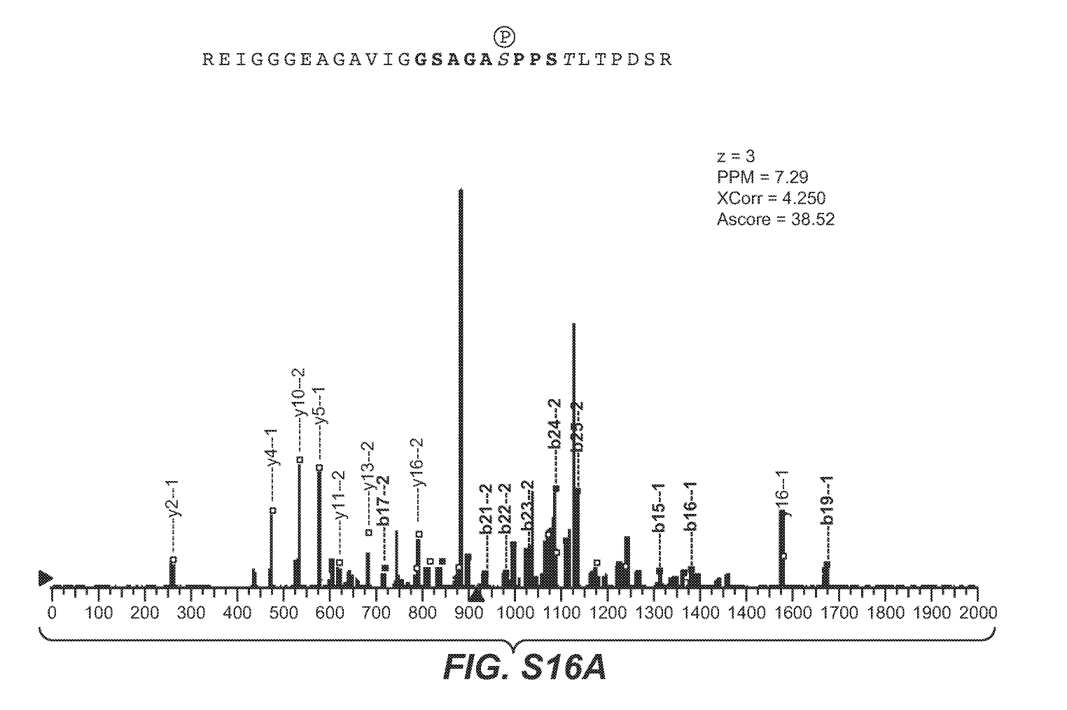

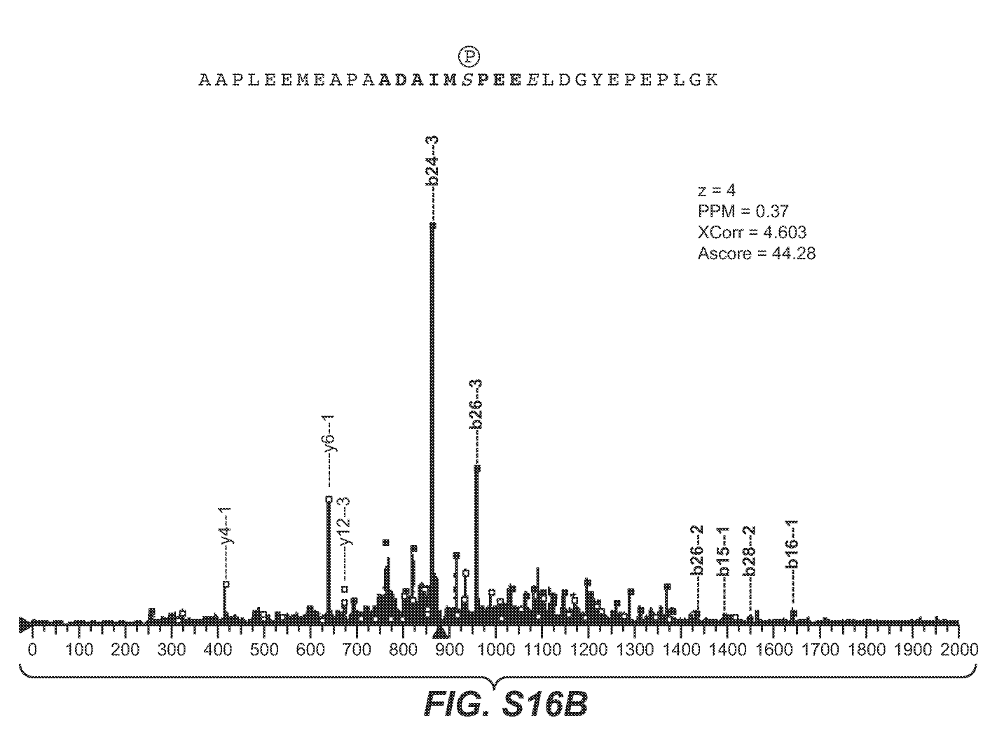

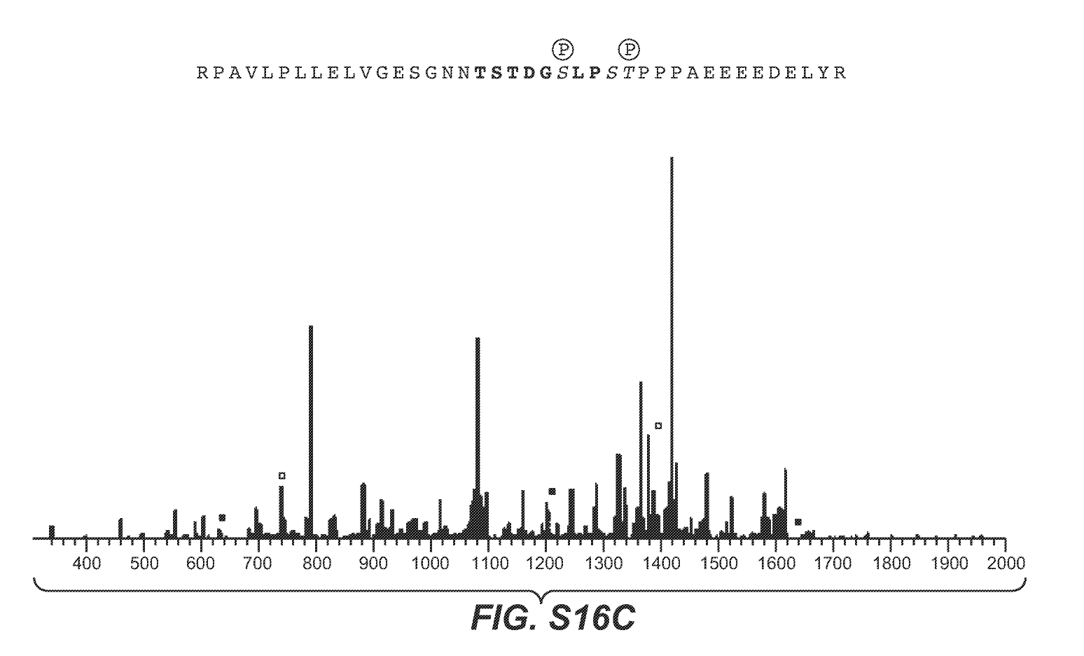

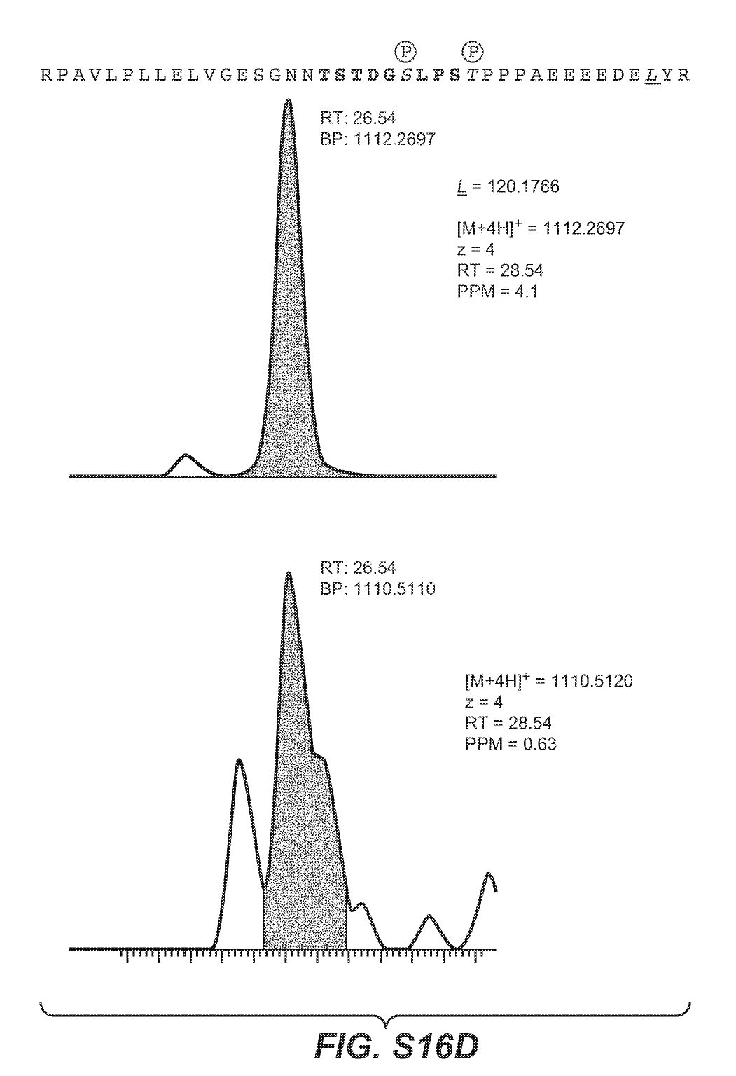

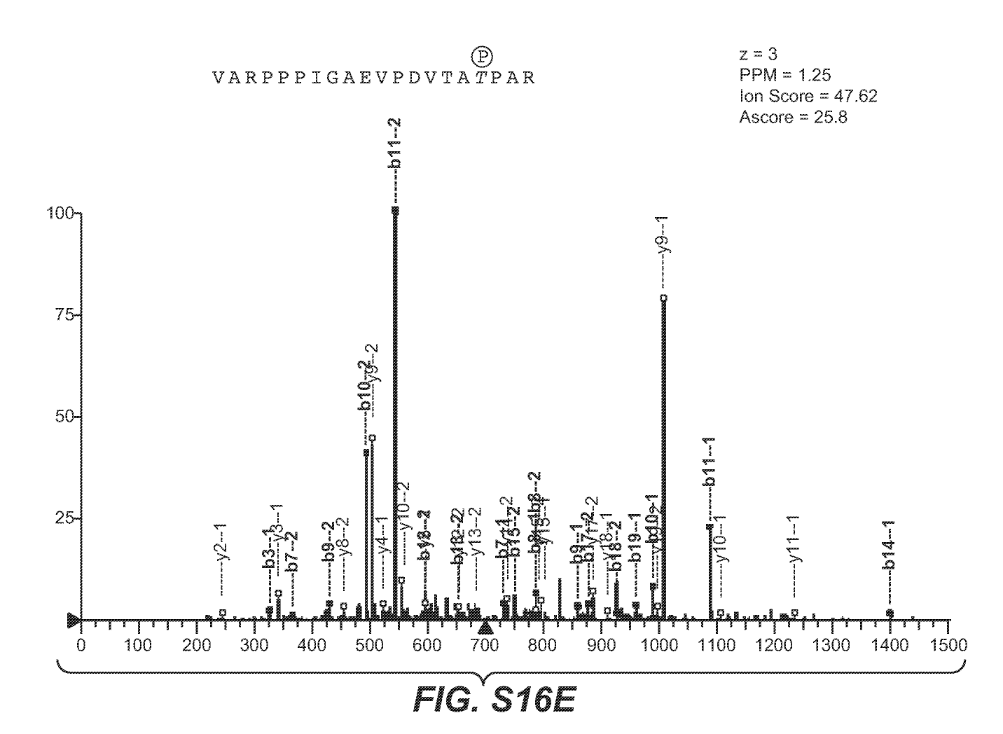

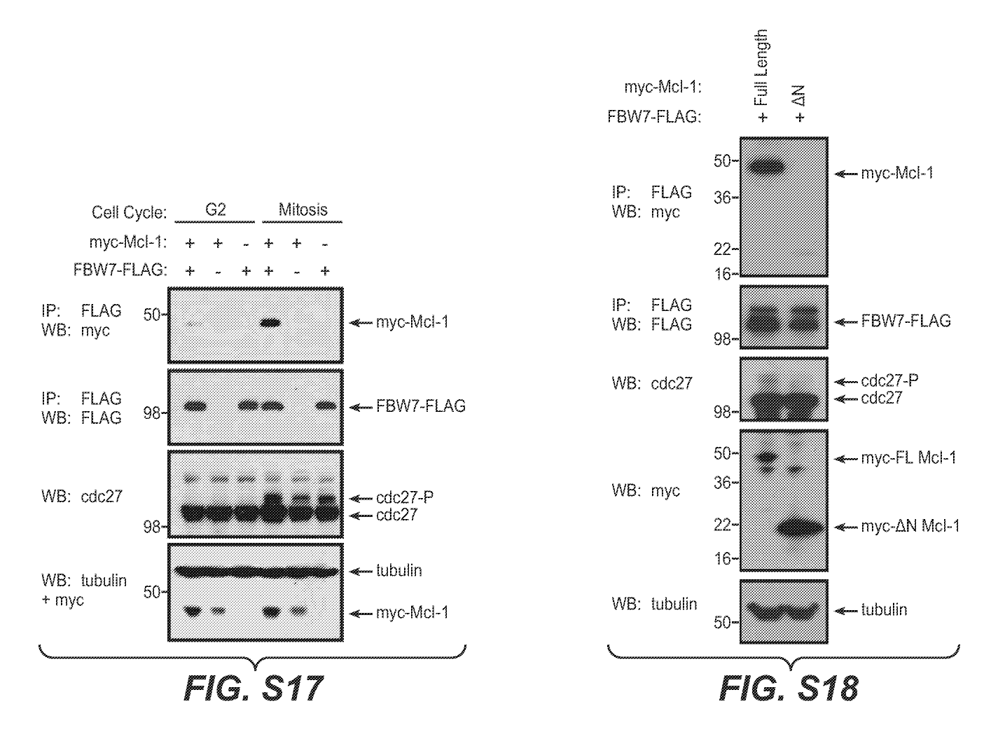

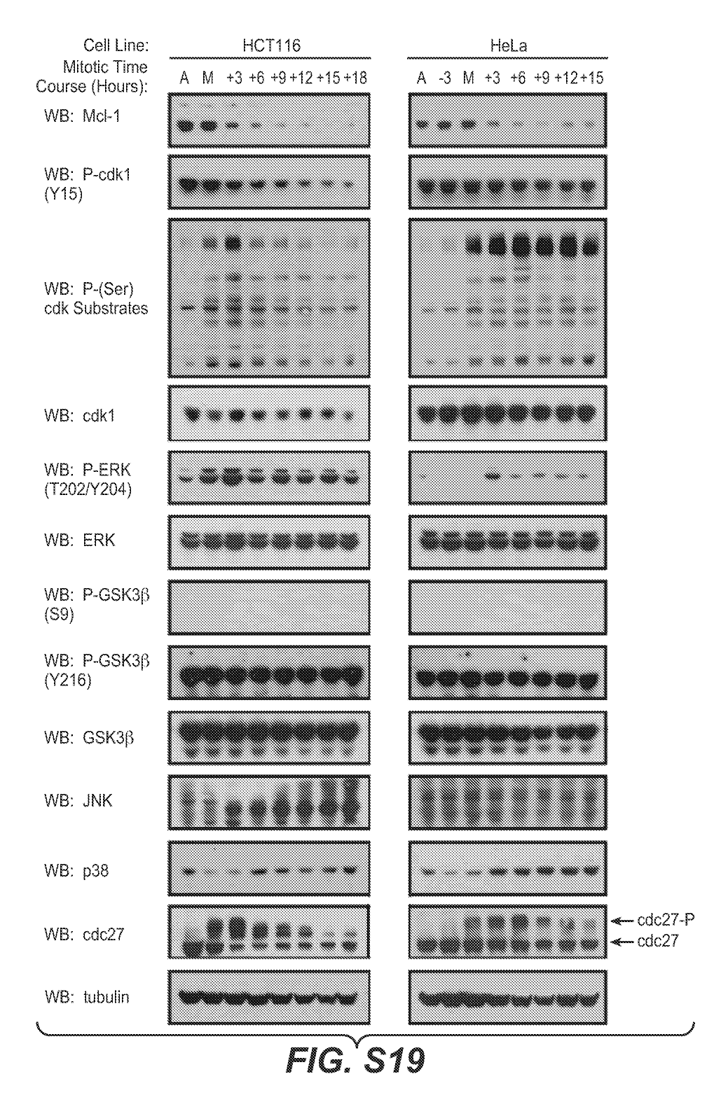

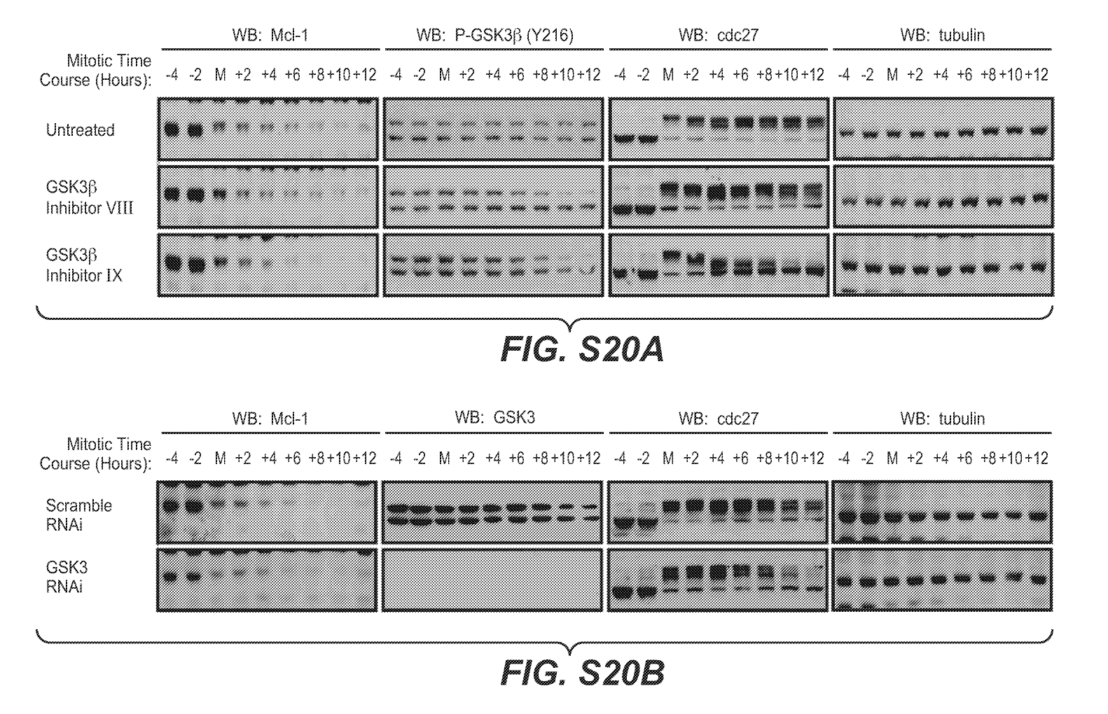

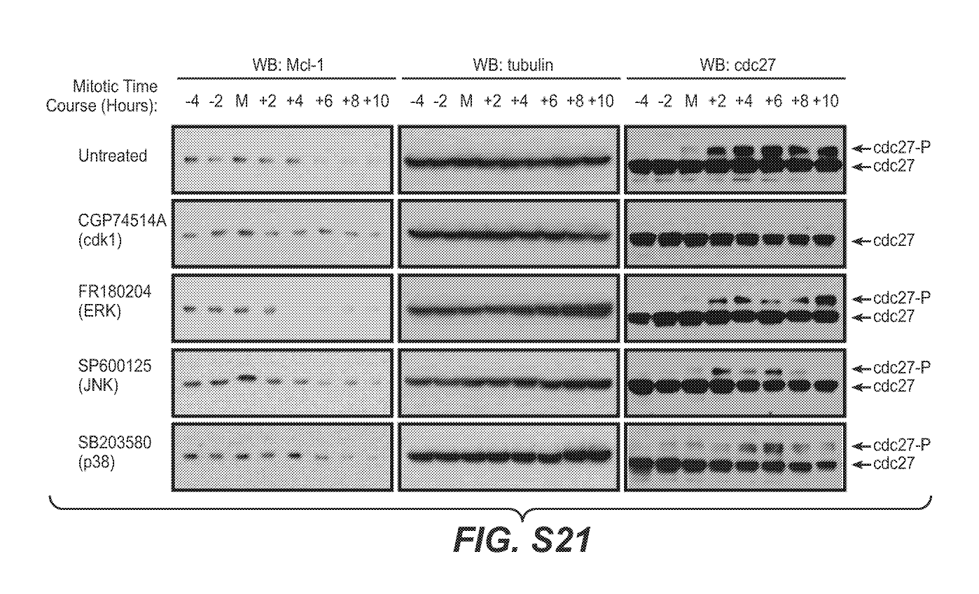

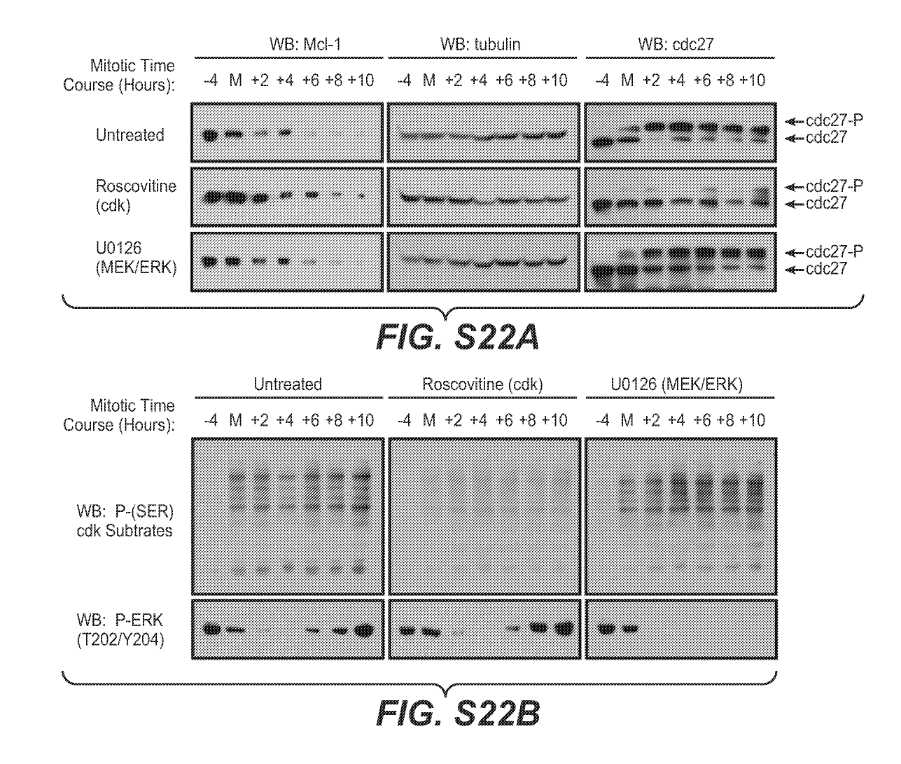

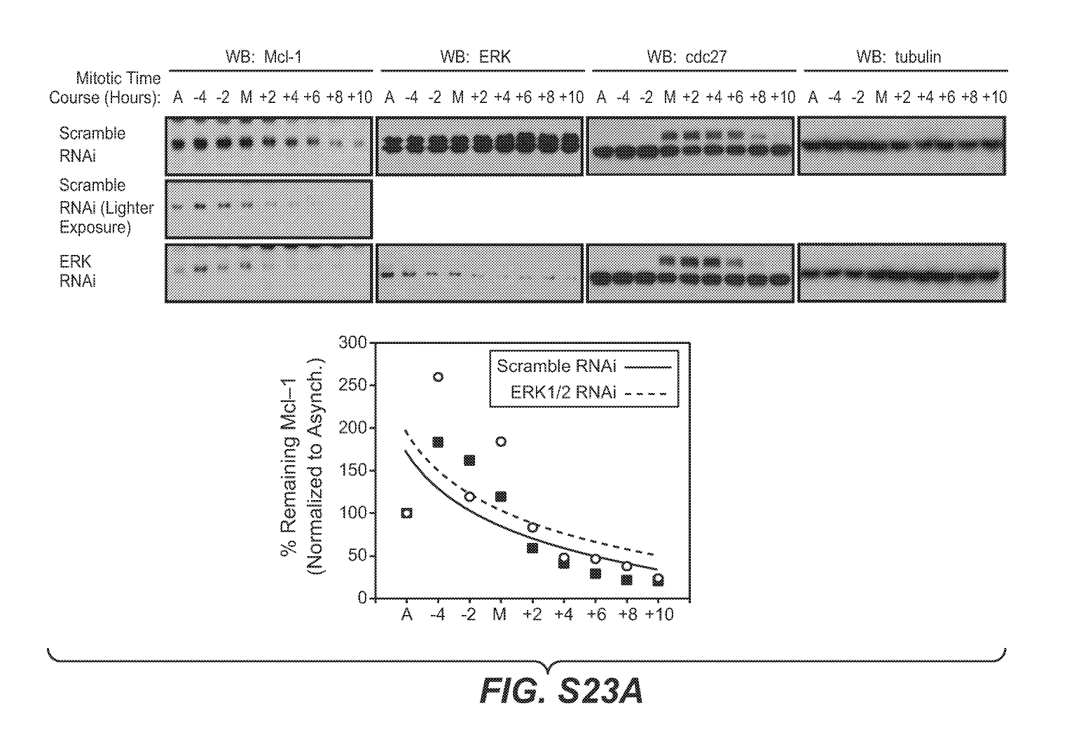

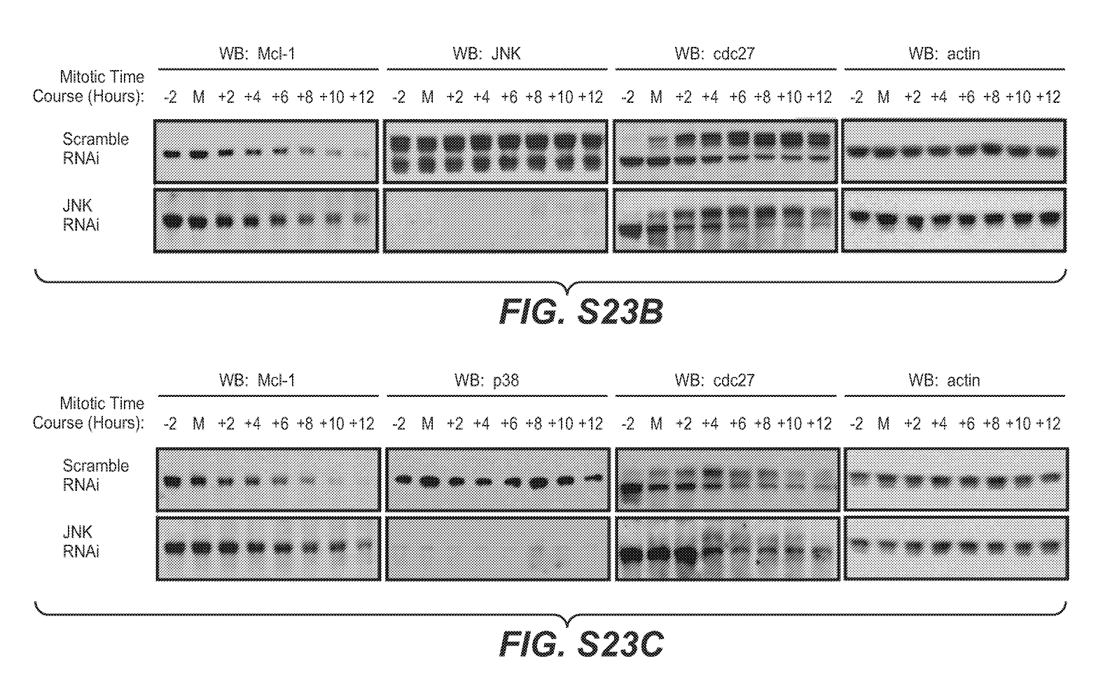

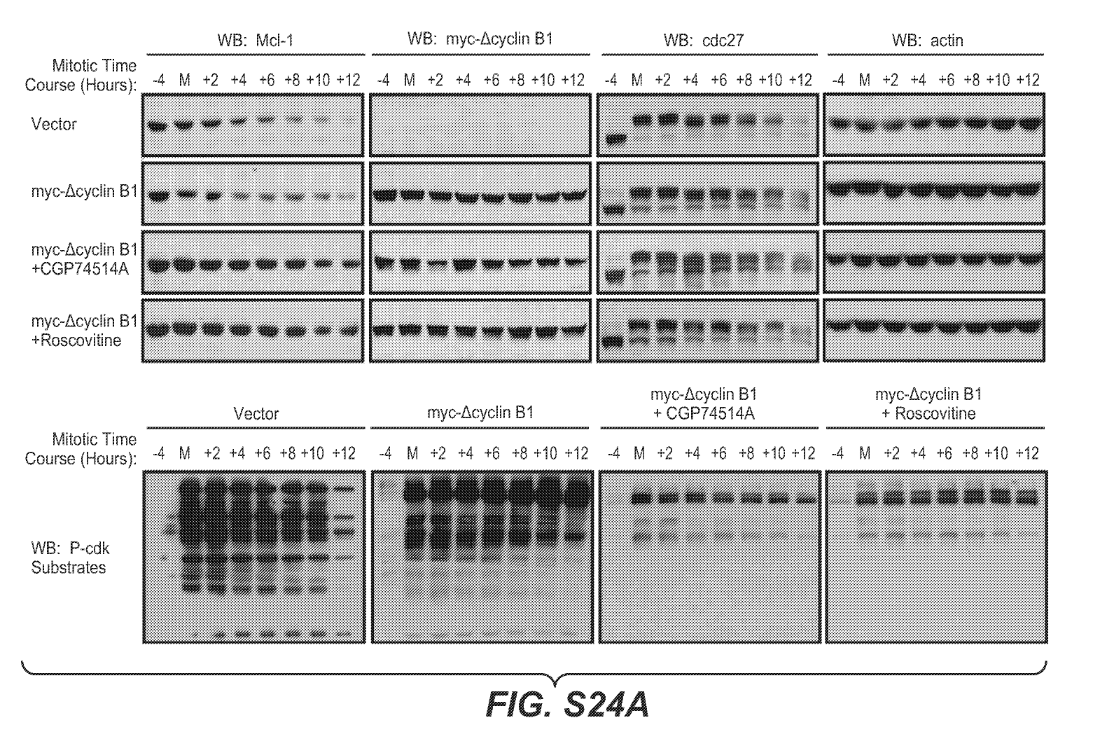

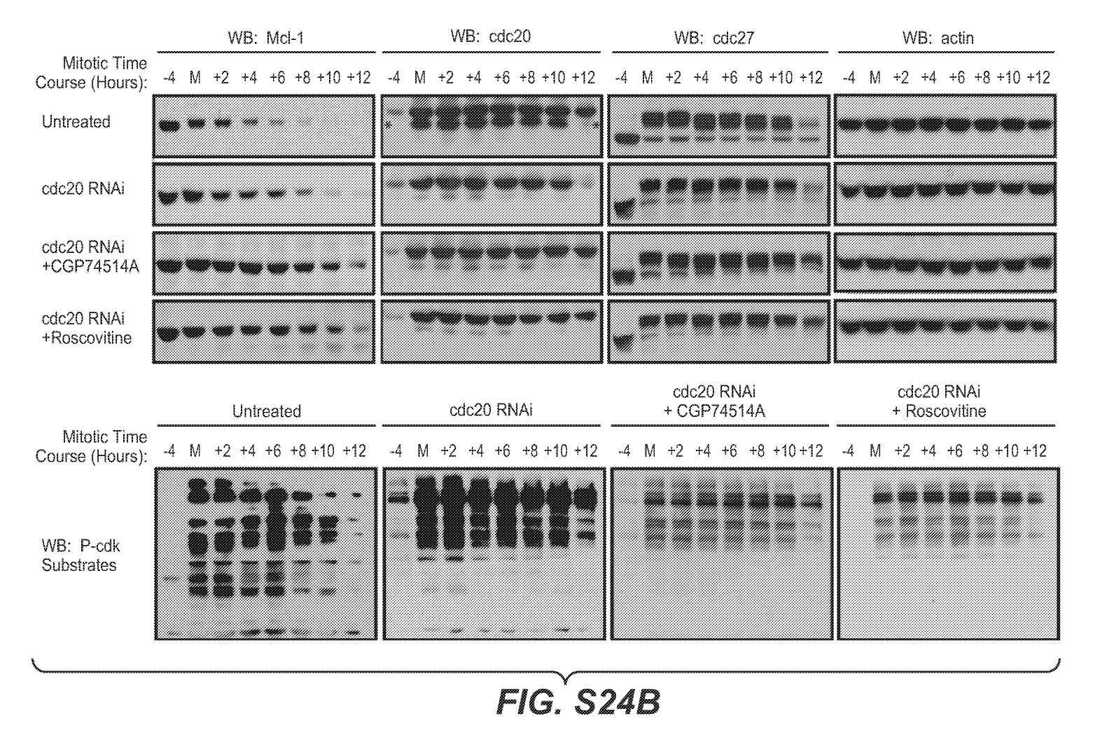

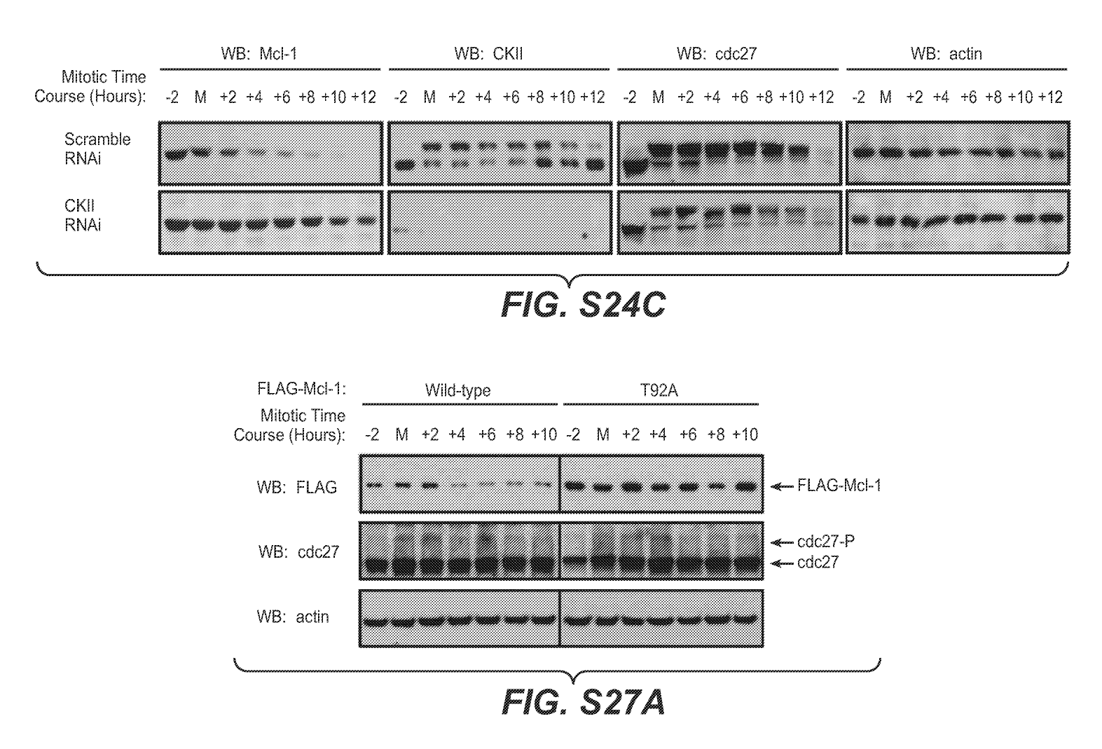

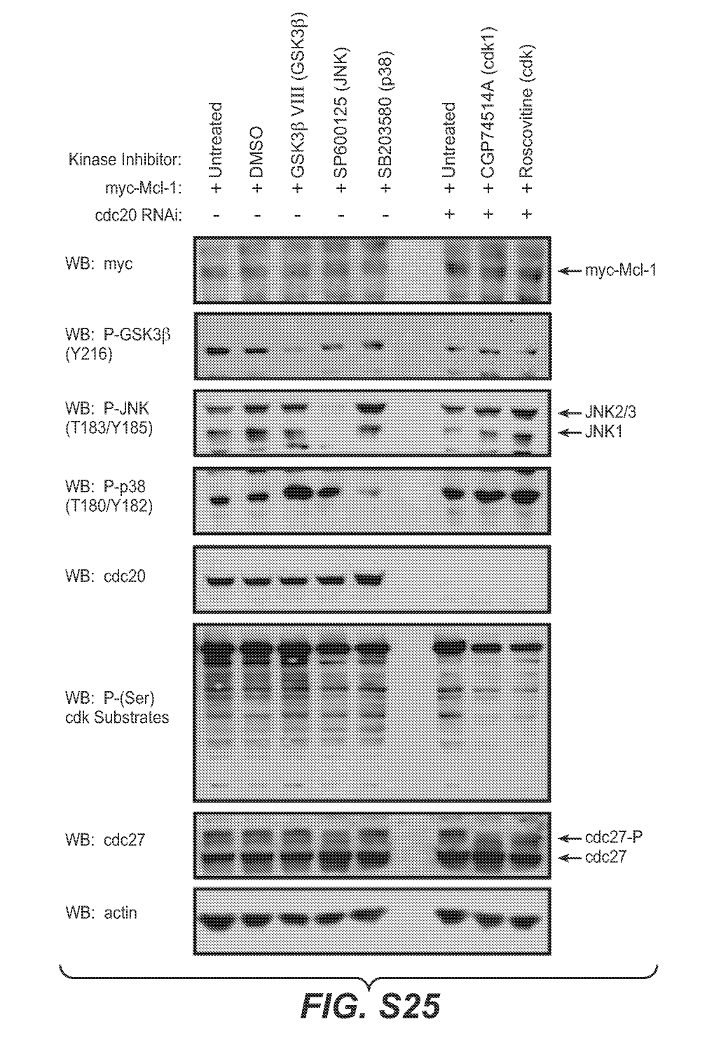

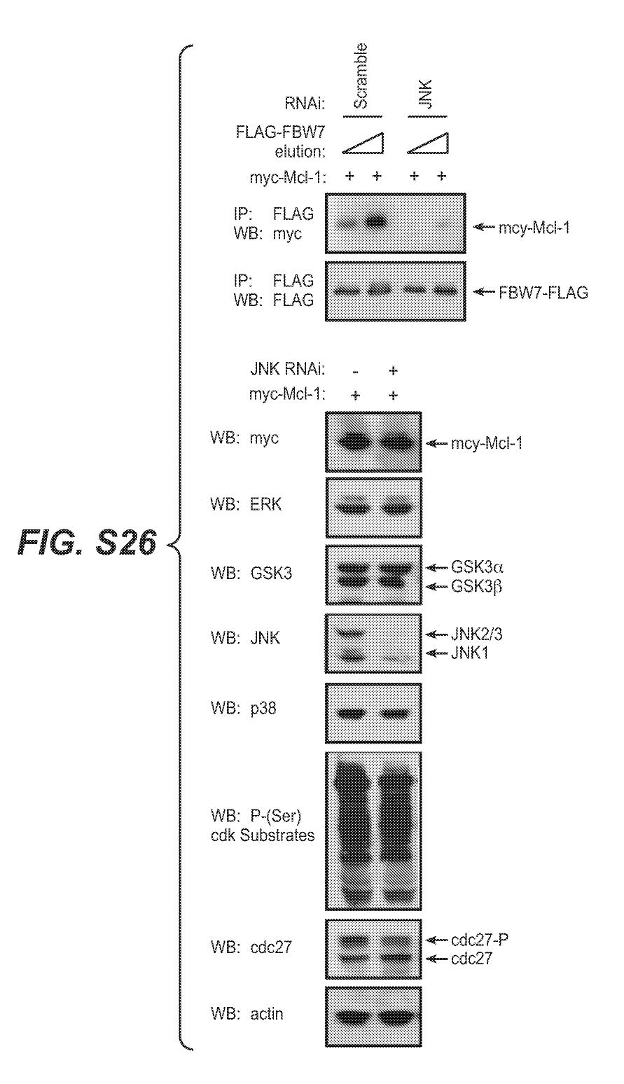

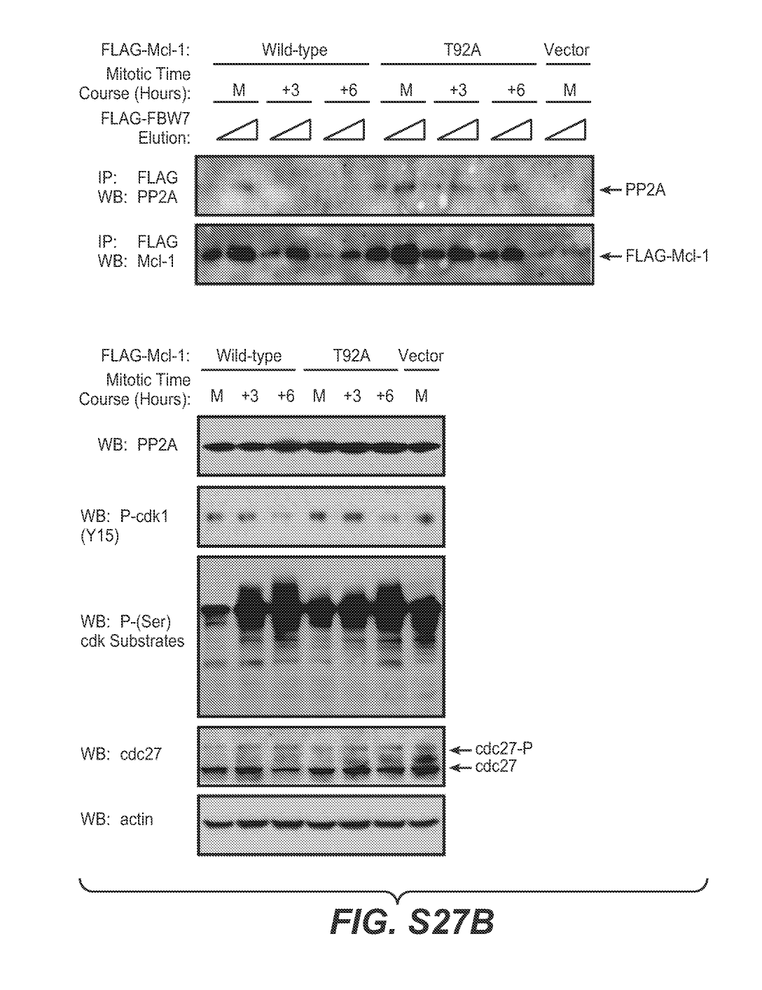

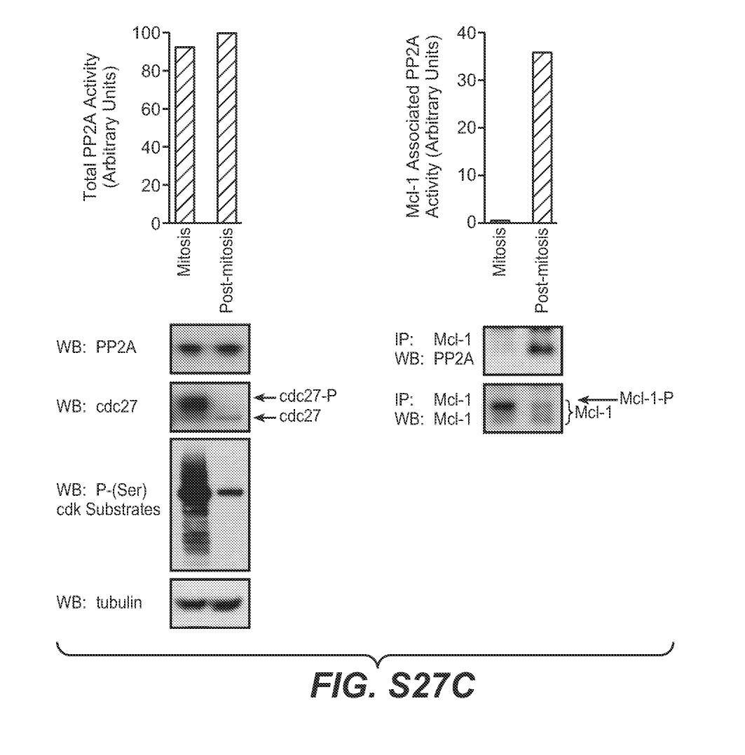

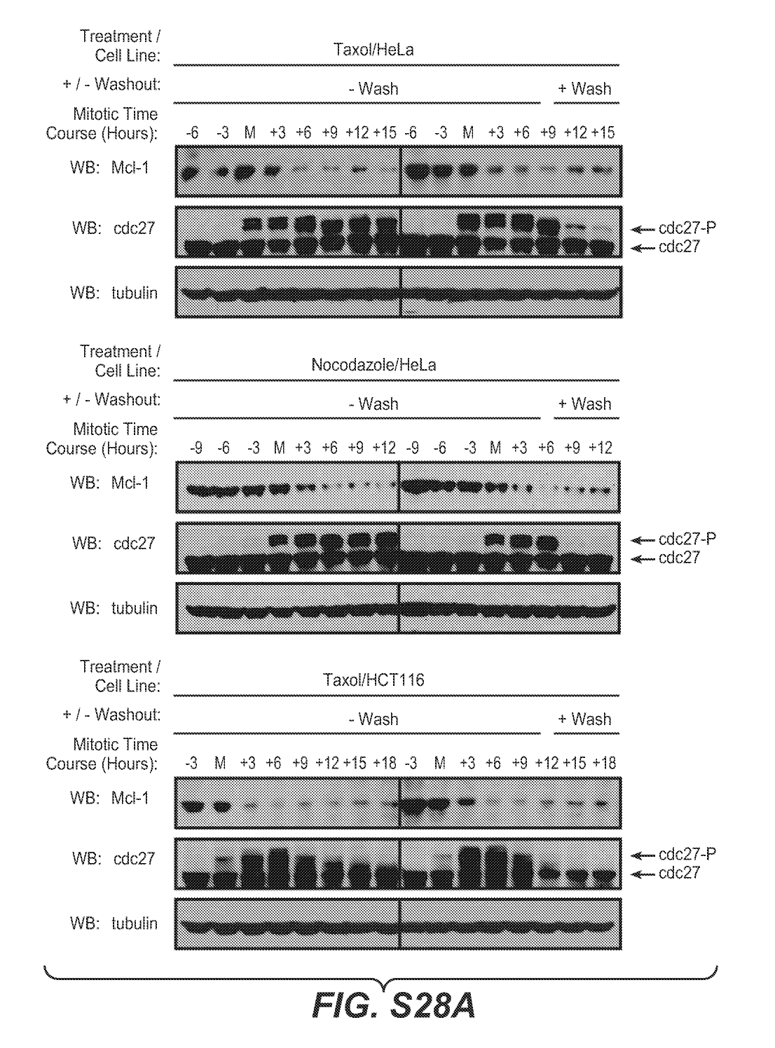

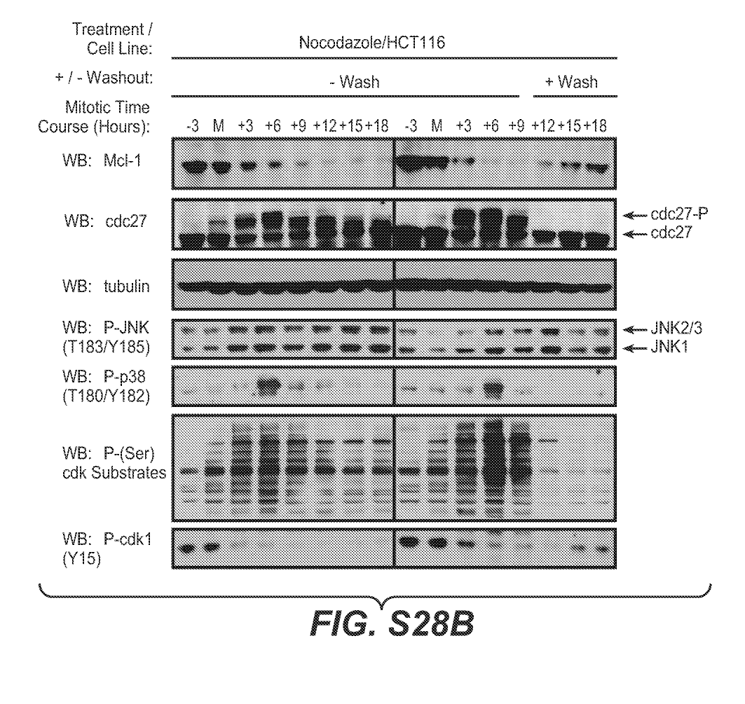

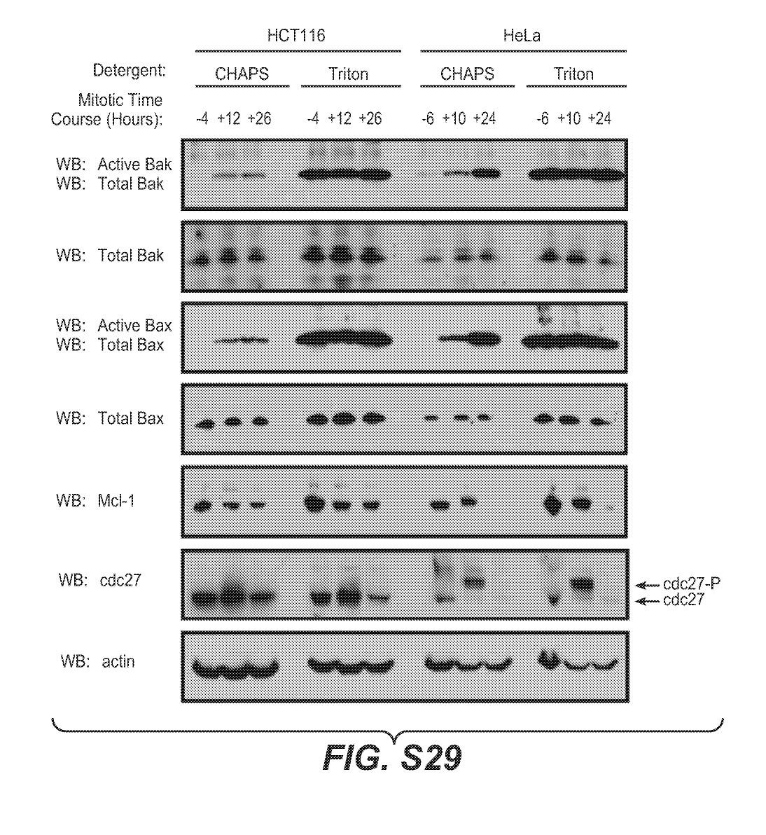

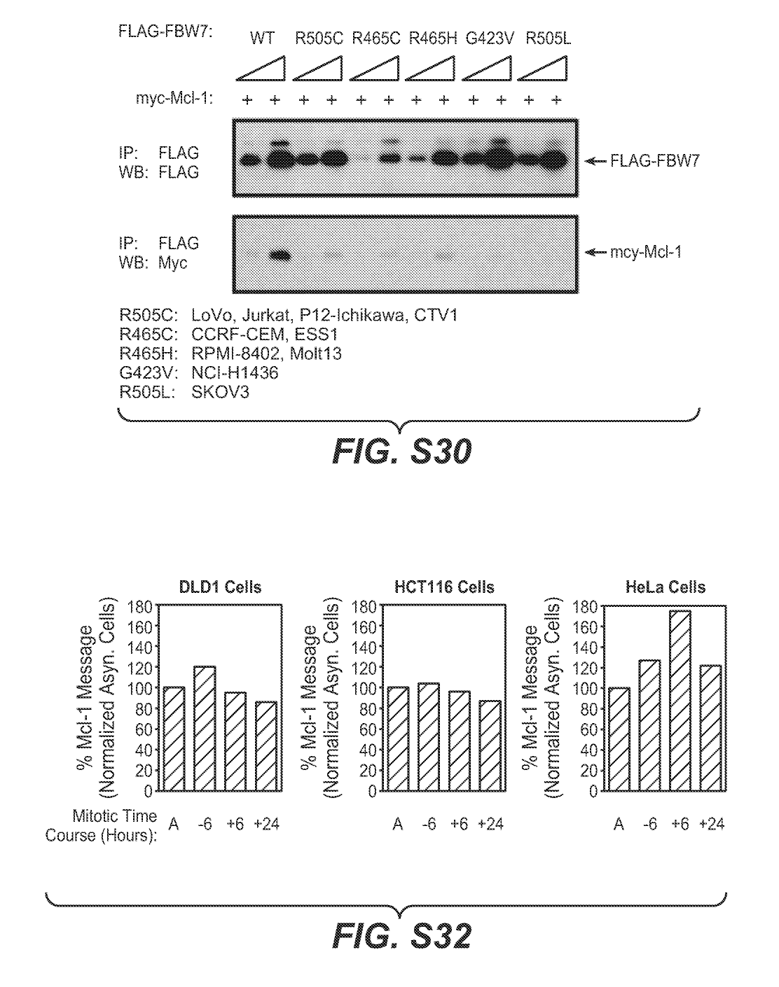

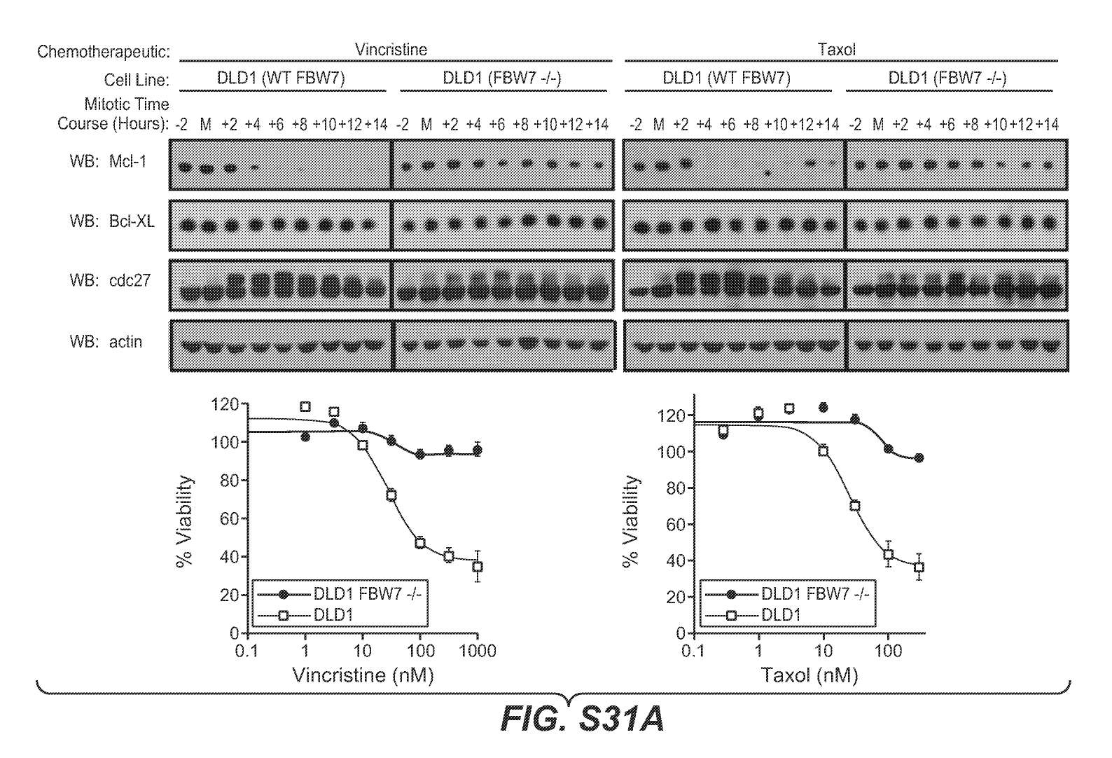

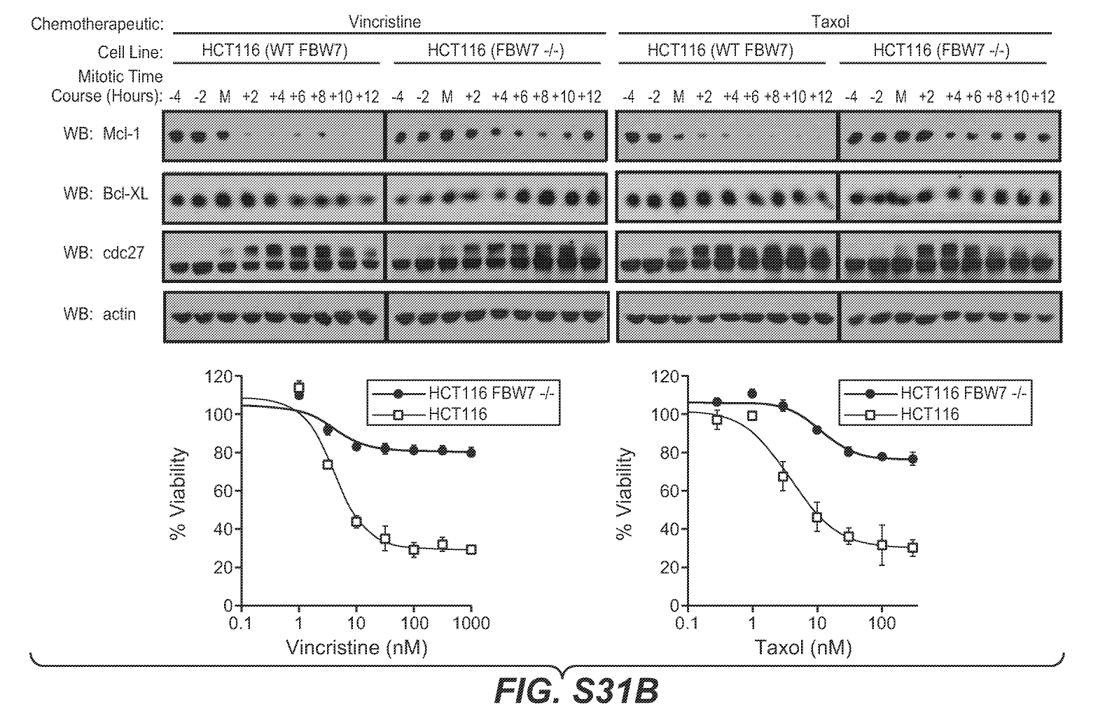

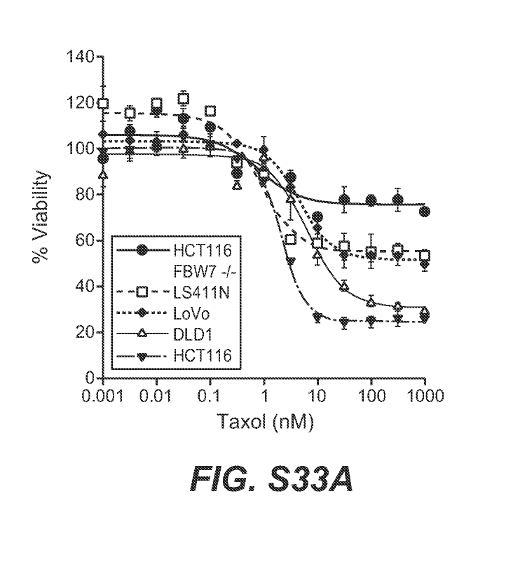

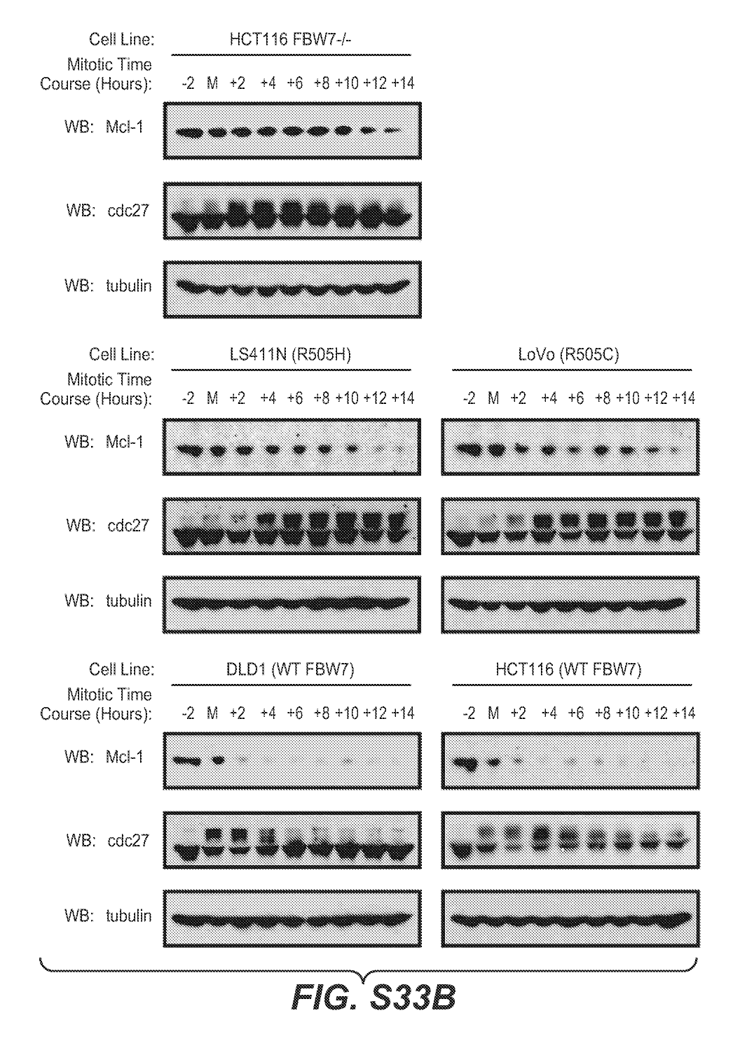

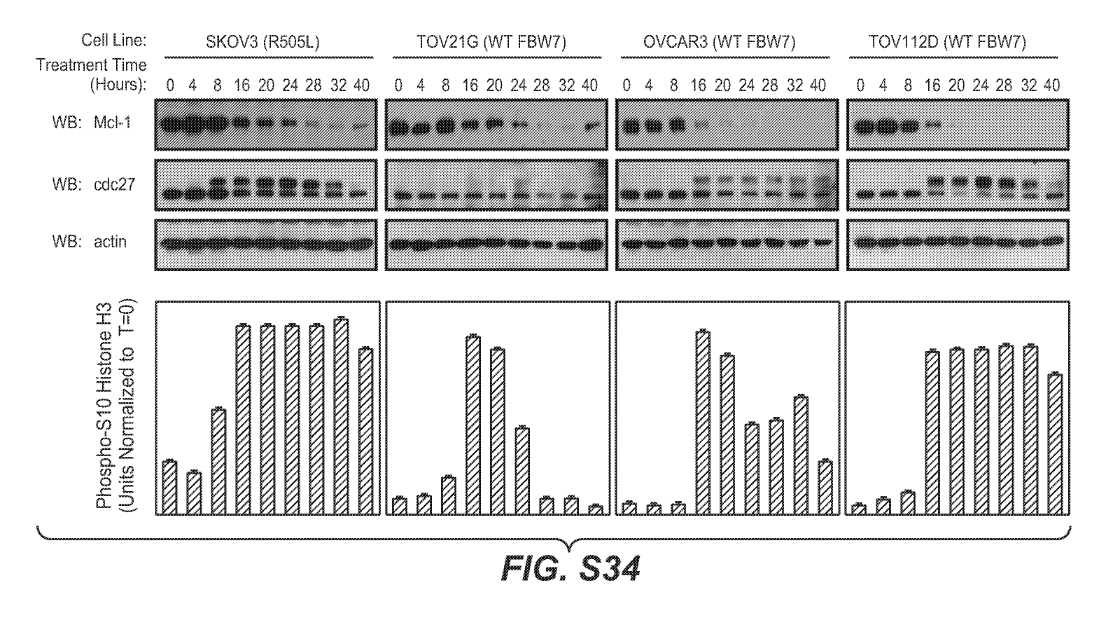

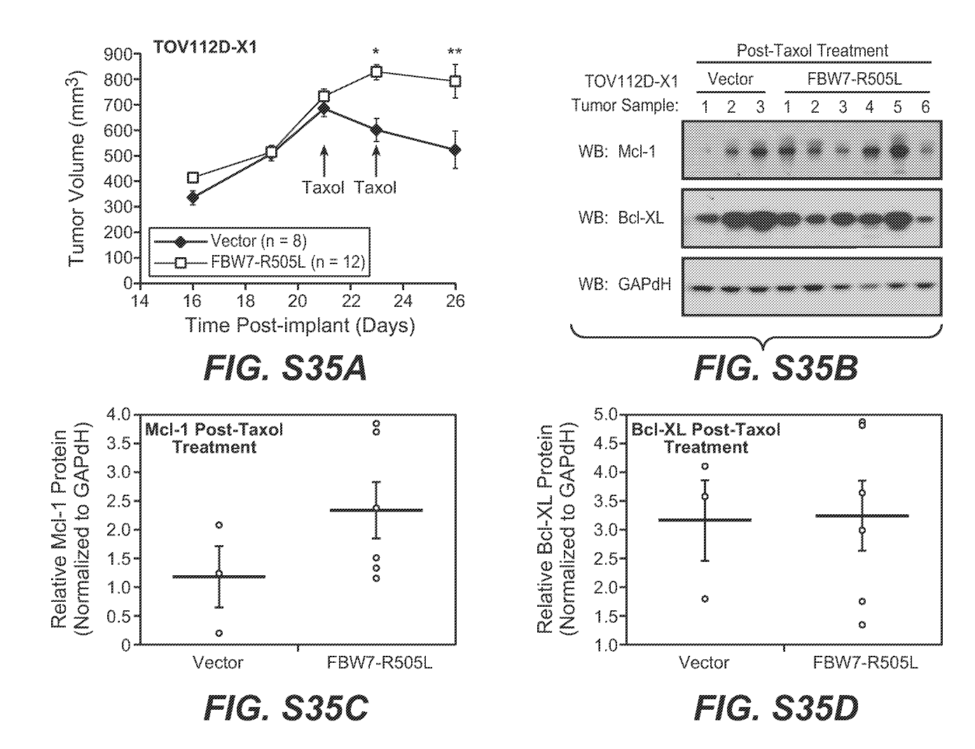

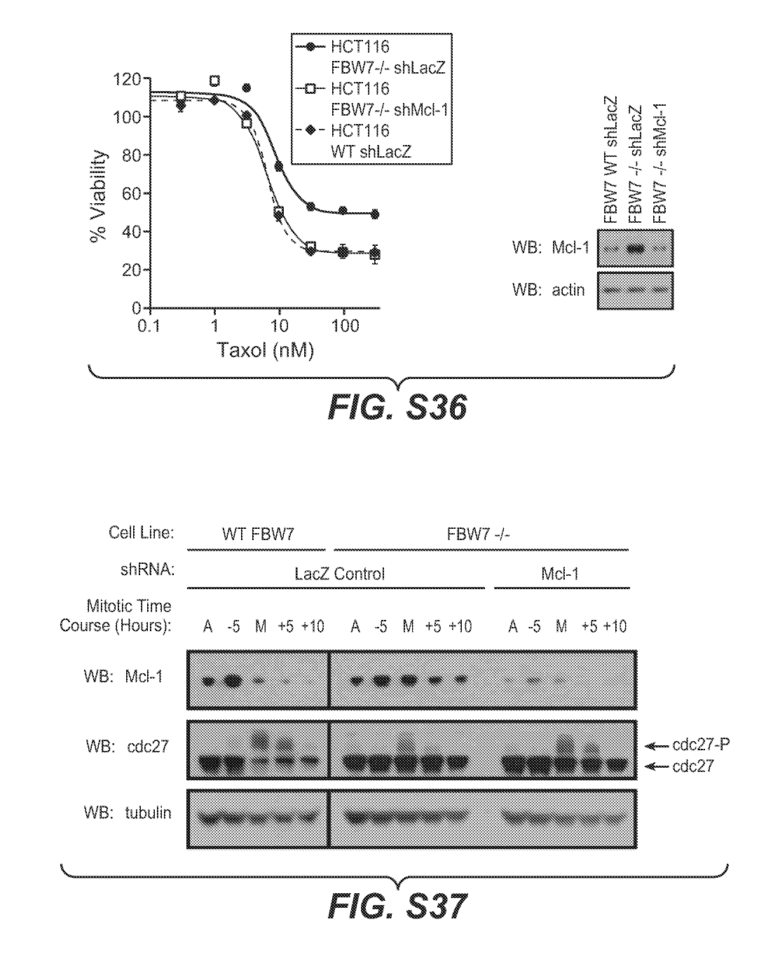

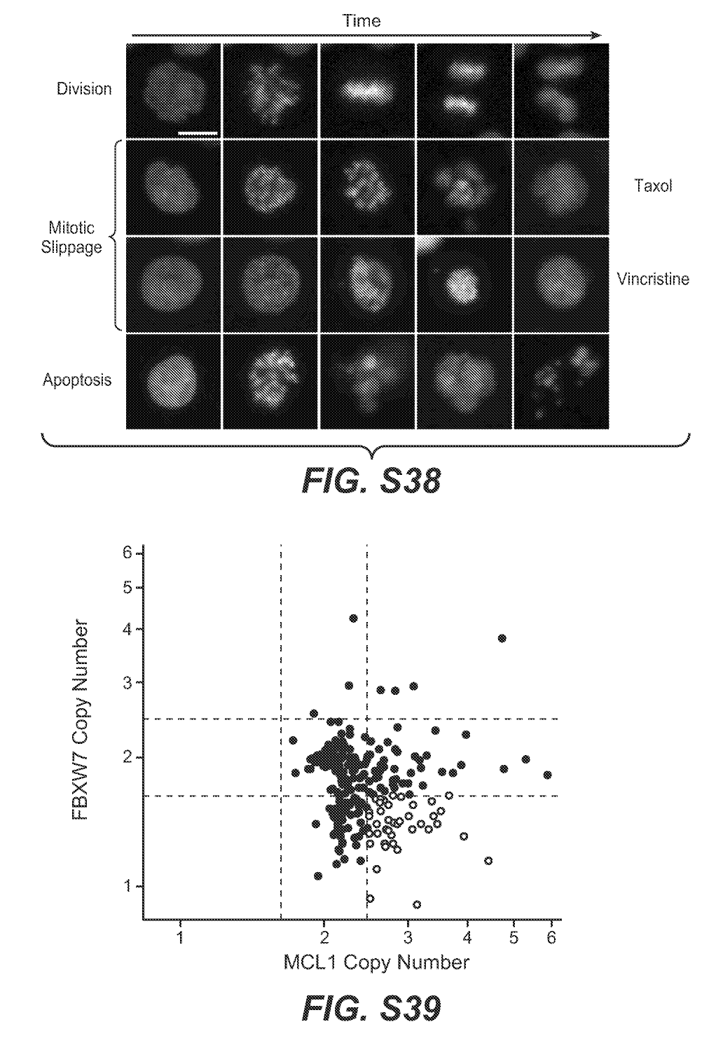

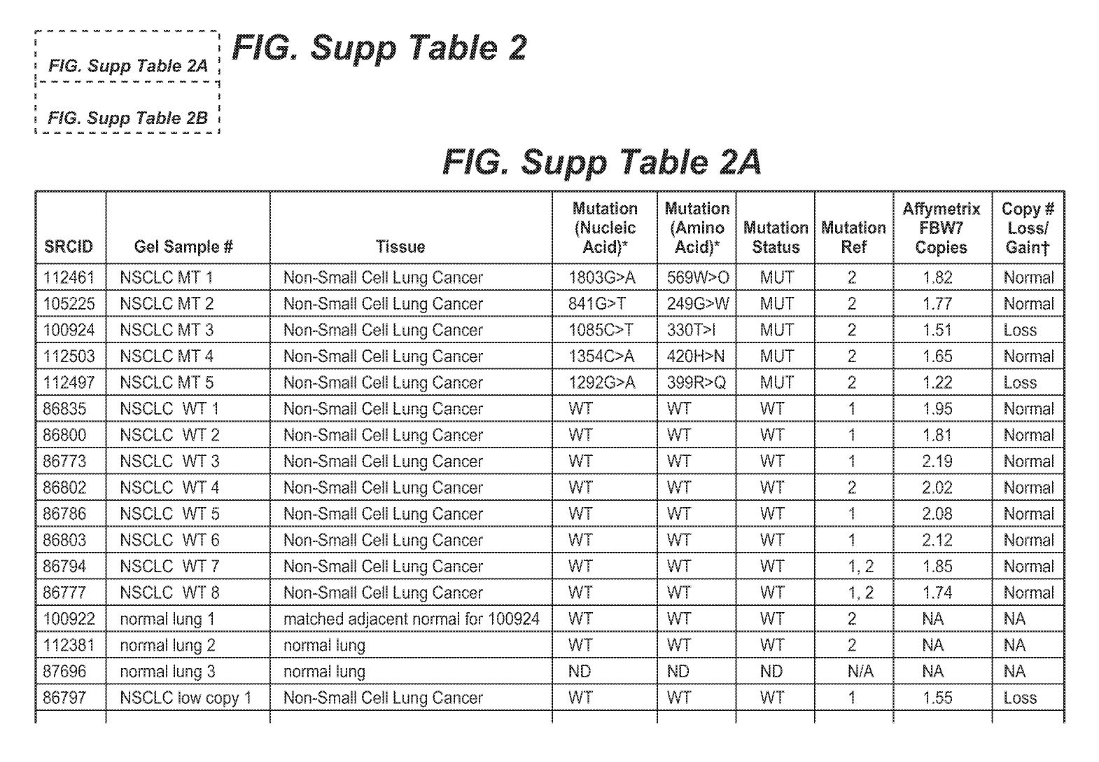

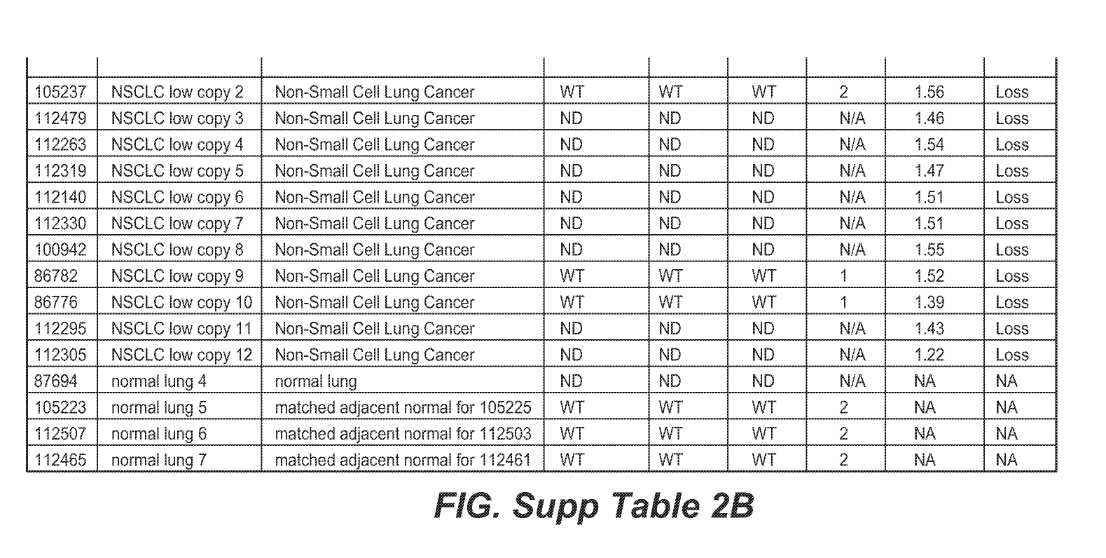

FIG. 14 shows the anti-tubulin antibody-drug conjugate, anti-CD79b-MC-vc-PAB-MMAE (ADC-MMAE) promotes mitotic arrest and Bcl family protein modulation in Granta-519 and WSU-DLCL2 NHL B-cell lymphoma cell lines, relative to a negative, non-specific binding antibody-drug conjugate control, anti-CD22 ADC.

These experiments show that Mcl-1 is degraded by tumor suppressor FBW7 in mitotic arrest upon treatment with anti-tubulin chemotherapeutic agents. When FBW7 is mutated, Mcl-1 is no longer degraded. Mcl-1 and FBw7 are useful pharmacodynamic (PD) biomarkers to monitor and predict therapeutic response to anti-tubulin chemotherapeutic agents.

### METHODS OF THE INVENTION

The methods of the invention include:

methods of diagnosis based on the identification of a biomarker;

methods of determining whether a patient will respond to a particular anti-tubulin chemotherapeutic agent;

methods of optimizing therapeutic efficacy by monitoring clearance of an anti-tubulin chemotherapeutic agent;

methods of optimizing a therapeutic regime by monitoring the development of therapeutic resistance mutations; and

methods for identifying which patients will most benefit from treatment with anti-tubulin chemotherapeutic agent therapies and monitoring patients for their sensitivity and responsiveness to treatment with anti-tubulin chemotherapeutic agent therapies.

The methods of the invention are useful for inhibiting abnormal cell growth or treating a hyperproliferative disorder such as cancer in a mammal (e.g., human). For example, the methods are useful for diagnosing, monitoring, and treating multiple myeloma, lymphoma, leukemias, prostate cancer, breast cancer, hepatocellular carcinoma, pancreatic cancer, and/or colorectal cancer in a mammal (e.g., human).

Cancers which can be treated according to the methods of this invention include, but are not limited to, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In order to use an anti-tubulin chemotherapeutic agent for the therapeutic treatment (including prophylactic treatment) of mammals including humans, an effective dose is formulated in accordance with standard pharmaceutical practice as a pharmaceutical composition with a pharmaceutically acceptable diluent or carrier in the form of a lyophilized formulation, milled powder, or an aqueous solution.

A typical formulation is prepared by mixing the anti-tubulin chemotherapeutic agent and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance or stabilized form is dissolved in a suitable solvent in the presence of one or more of the excipients described above. The anti-tubulin chemotherapeutic agent is typically formulated into pharmaceutical dosage forms to provide an easily controllable dosage of the drug and to enable patient compliance with the prescribed regimen.

### EXAMPLES

### Methods Summary

The viability of cancer cell lines, and MEFs in which genes encoding IAPs had been knocked out, was analysed by using the CellTiter-Glo Luminescent Cell Viability Assay® (Promega). Cells were treated in triplicate with anti-tubulin agents for the indicated times, using dimethylsulphoxide treatment as a control. The viability of BCL2-family-member-null MEFs was analysed by propidium iodide staining, as described previously (Chen, L. et al. (2005) Molecular cell 17:393-403), after treatment with anti-tubulin agents for 48 h. Cell synchronization was achieved by culture either in serum-free medium for 12-16 h or in medium containing 2 mM thymidine for 18-24 h, release from the thymidine block with three washes in PBS, followed by culture for 8-12 h in complete growth media (compositions are described in the Supplementary Information). Cells then underwent a second thymidine block for 16-20 h, three further washes in PBS and release into complete medium containing the indicated reagents. To block MCL1 degradation, 25 mM MG132 was added as cells entered mitotic arrest, as assessed by visual inspection. See the Examples for full methods.

### Plasmids and Reagents

HA-CUL1 was used as a template to generate dominant negative HA-CUL1 (residues 1-428). Human FLAG FBW7-alpha was synthesized and cloned into a pRK vector by Blue Heron. Full-length FBW7-alpha and FBW7-alpha delta F-box (with residues 284-324 deleted) were subcloned into pcDNA3-myc/his (Invitrogen). Point mutations in FBW7-alpha (R505C, R465C, R465H, G423V, R505L) were generated by site-directed mutagenesis. FLAG FBW7-beta was made by swapping exon 1 of FLAG FBW7-alpha with exon 1 of the FBW7-beta isoform. GFP-H2B viral supernatant was purchased from Invitrogen. Mcl-1 shRNAs were cloned into the doxycycline-inducible pHUSH retroviral system as described (Gray, D. C. et al. (2007) BMC biotechnology 7:61). The FLAG Mcl-1 construct has been described (Willis, S. N. et al. (2007) Science (New York, N.Y 315:856-859). Mcl-1 phosphomutants (S64A/T68A, S121S/E125A, 159A/T163A, and 4A=S64A/S121A/S159A/T163A were synthesized and cloned into pcDNA3 vectors by Blue Heron and subcloned into pCMV-Tag3B (Stratagene) and pMXs. IP22, and the T92A phosphomutant was generated by site-directed mutagenesis. Myc epitope-tagged cyclin B1 delta-85 (myc-Δcyclin B1) was cloned in a pCS2 vector. Antibodies to the following proteins were purchased from the indicated vendors: monoclonal Mcl-1 (clone 22), monoclonal GSK3β (pY216) (clone 13A), polyclonal Bcl-X and Mcl-1 antibodies (BD Biosciences); monoclonal anti-Bak (Ab-1) antbody (Calbiochem); monoclonal anti-Bax YTH-6A7 anitbody (Trevigen); anti-PP2A clone 1D6 (Upstate); human Mcl-1, Phospho-(Ser) cdk substrate antibody, cdk1, Phospho-cdk1 (Tyr15), cyclin B1, p38 MAPK, Phospho-p38 MAPK (Thr180/Tyr182) (#9211), rabbit monoclonal GSK-3β (27C10), Phospho-GSK-3β (Ser9) (5B3), GSK-3α/β (D75D3) rabbit MAb, p44/42 MAPK (Erk1/2) (137F5), Phospho-p44/42 MAPK (Erk1/2) (Thr202/Tyr204) (D13.14.4E) XP™, SAPK/JNK (56G8), Phospho-SAPK/JNK (Thr183/Tyr185) (#9251), monoclonal Cyclin E (HE12), polyclonal anti-cdc20 (#4823), polyclonal CKII-alpha (#2656), polyclonal Bad, Bax, Bim and Puma antibodies (Cell Signaling Technology); Bcl-2 (clone Bcl-2-100), polyclonal CUL1 and ROC1 antibodies (Zymed); FLAG monoclonal antibody and agarose (clone M2), polyclonal Bak and HA-7 HA-HRP (Sigma); Noxa (clone 114C307) (Novus Biologicals); c-Myc (clone 9E10), cdc27 (clone H300), ubiquitin (clone P4D1), and actin-HRP (Santa Cruz Biotech); polyclonal SKP1 antibody (New England Biolabs); HA high-affinity matrix (clone 3F10) (Roche); β-tubulin (clone DM1B) (MP Biomedicals); GAPdH (clone 1D4) (Stressgen). Kinase inhibitors were used at indicated concentrations and purchased from the following vendors: CGP74514A (cdk1)(2 μM or 4 μM), FR180204 (ERK)(2 μM), GSK3β VIII (GSK3β)(2 μM or 25 μM), GSK3β IX (GSK3β)(25 μM), SP600125 (JNK)(25 μM), SB203580 (p38)(2 μM or 2.65 μM) from Calbiochem; Roscovitine (cdk)(2.5 μM) from Sigma; U0126 (MEK/ERK)(10 μM) from Promega.

### Cell Lines, Cell Culture, and Transfections

TOV112D, SKOV3, LoVo, LS411N (American Type Culture Collection) and TOV112D-X1 cells were cultured in RPMI 1640 with 10% fetal bovine serum and 1× L-Glutamine. TOV112D-X1 cell line was generated by implanting TOV112D into NCR.nude mice, excising the xenograft tumor, isolating and culturing the tumor cells. Parental HCT116 and DLD1 (American Type Culture Collection) and HCT116 and DLD1 FBW7−/− (Horizon Discovery) were cultured in McCoy's 5A with 10% fetal bovine serum and 1× L-Glutamine. OVCAR3, TOV21G cells (American Type Culture Collection) were cultured in RPMI 1640 with 20% fetal bovine serum and 1× L-Glutamine. The FBW7 status of all patient-derived colon and ovarian cancer cell lines was confirmed for the reported FBW7 status (http://www.sanger.ac.uk/genetics/CGP) by in-house DNA sequencing (data not shown). Plat-A cells were maintained in high glucose DMEM with 10% fetal bovine serum and 1× L-Glutamine containing blasticidin (10 μg/ml) and puromycin (1 μg/ml). cIAP1−/−, cIAP2−/− and XIAP−/− MEFs were described previously (Varfolomeev, E. and Vucic, D. (2008) Cell cycle (Georgetown, Tex. 7, 1511-1521; Vince, J. E. et al. (2007) Cell 131, 682-693). Factor Dependent Myeloid (FDM) cell lines were generated by infecting E14.5 fetal liver single suspensions with a HoxB8 expressing retrovirus and cultured in the presence of high levels of IL3, as previously described (Ekert, P. G. et al. (2004) Journal of cell biology 165:835-842). BAX−/− mice were obtained from the Jackson Laboratory; BAK−/− mice and BCL-X−/−, BCL-2−/− and BCL-W−/− mice were generated as described (Ekert, P. G. et al. (2004) Journal of cell biology 165:835-842). All mice used were of C57BL/6 origin or have been backcrossed (>10 generations) to this genetic background. E1A/RAS immortalized MEFs were generated from E12.5-E14.5 embryos after retroviral infection (at passage 2-4) with pWZLH.12S[E1A] and pBabePuro.H-Ras. Pools of cells from single donors of each genotype were selected by incubation with puromycin (Sigma) and hygromycin B (Roche) for 1 week. Other MEFs were generated from E13-14.5 embryos and immortalized (at passage 2-4) with SV40 large T antigen (LTA) or 3T9 methods as described (Ekert, P. G. et al. (2004) Journal of cell biology 165:835-842). WT and all Bcl-2 family KO MEFs (Bax−/−/Bak−/−, Bclw−/−, Bcl2−/−, Mcl1−/− and BclX−/−) were cultured in DMEM supplemented with 10% fetal calf serum (FCS), and in some cases also with 250 μM L-Asparagine and 50 μM 2-mercaptoethanol. For transient transfections, Plat-A cells were transfected with Fugene HD (Roche), HCT 116 and HeLa cells were transfected with Lipofectamine LTX or Lipofectamine 2000 (Invitrogen), and MEFs were transfected with siRNA using Lipofectamine RNAiMAX reagent (Invitrogen) as recommended by the respective manufacturers. For retroviral transductions, culture supernatant from Plat-A cells transfected with the indicated expression vectors were added to the cells in the presence of 8 μg/ml of polybrene for 48 hours. Appropriate selection reagent(s) were then added to select stable cell lines.

### Western Blotting and Immunoprecipitations

Western blotting was performed essentially as described (Wertz, I. E. et al. (2004) Science (New York, N.Y 303:1371-1374). In brief, cells were lysed in corrected FLAG elution buffer (CFEB) (19.17 mM Tris (pH 7.5), 916.7 μM MgCl2, 92.5 mM NaCl and 0.1% Triton X-100) with protease and phosphatase inhibitors; in some cases 6 M urea was added. Cleared lysates were quantitated and equal amounts of proteins were reduced, alkylated, separated by SDS-PAGE, and transferred onto PVDF membranes (Invitrogen) following standard procedures. Western blotting was performed as recommended by the respective antibody manufacturers. Patient tissue and xenograft samples were lysed in 5× volume of CFEB with protease inhibitors using Fast prep 24 (MP Biologicals). Tissue lysates were cleared and 40 μg total protein was prepared for western blotting analysis as described above. Immunoprecipitations were performed with the indicated antibodies as described (Willis, S. N. et al. (2007) Science (New York, N.Y 315:856-859; Wertz, I. E. et al. (2004) Science (New York, N.Y 303:1371-1374). PP2A activity was performed on PP2A or Mcl-1 immunoprecipitates as recommended by the manufacturer (Upstate)

### FACS Analysis

HCT116 WT or HCT116 FBW7−/− cells expressing shLacZ or shMcl-1 constructs were treated with 200 nM vincristine and harvested at designated time points. Cells were fixed and permeabilized with 70% ethanol in PBS and stored at −20° C. prior to staining Cells were stained with 50 ug/mL of Propidium Iodide plus 60 units of RNase A and incubated for 2 hours in the dark at room temperature and then analyzed on a FACS Calibur® (BD Biosciences). The fraction of polyploid cells with >4N chromosomal content was determined with Cell Quest Pro® software (BD Biosciences).

### Microscopy

HCT116 parental or FBW7−/− cells expressing shLacZ or shMcl-1 were plated at 15,000-30,000 cells per well in 96-well μ-plates (ibidi GmbH) and infected with GFP-H2B baculovirus (Invitrogen) 24 hours prior to adding paclitaxel or vincristine. Cells were imaged live at 37° C. with 5% CO2 using a Nikon TiE® microscope with a Cool Snap® CCD camera (Roper Scientific) and a Plan Apo VC 20× 0.75 NA objective. Three images with 6 μm z-steps were acquired for each position every 10 minutes for 43 hours. Mitotic fate was analyzed manually using NIS-Elements software (Nikon) and numerical data was complied and statistically analyzed using Excel (Microsoft). Fifty mitotic cells were analyzed for each condition and p-values were calculated for the change in the number of cells that exited mitosis or entered apoptosis using the one-tailed Fisher's exact test.

### Ovarian Tumor Xenografts In Vivo

Wild-type FBW7 TOV112D-X1 ovarian cancer cells expressing either an empty vector (vector) or the R505L point mutant (FBW7-R505L) were resuspended in Matrigel® (BD Biosciences) at a density of 1×108 cells/mL, and 10 mL Matrigel® grafts containing 1×10⁶ cancer cells were implanted under the kidney capsule of 8-week-old athymic nu/nu mice (Harlan Sprague Dawley). Only one graft was implanted per mouse. Once tumors became palpable on the kidney surface, tumor growth was assessed three times per week via caliper measurements of the entire kidney volume (0.523×length×width×height). On day 21 post-implant, when tumors reached an average volume of 700 mm³, paclitaxel (APP Pharmaceuticals) was administered to both FBW7-WT and FBW7-R505L tumor groups via intravenous tail vein injection at 20 mg/kg in 5% dextrose water. Paclitaxel administration was repeated on day 23 post-implant. Statistical differences were evaluated using a two-tailed Student's t-test. P values of less than 0.05 were considered significant.

### Quantitative Real-Time PCR Assay

Total RNA from cell lines was isolated using Qiagen RNeasy mini kit (Qiagen) and treated with DNase (Qiagen) as recommended by the manufacturer. Primers and probes were designed:

FBW7 primer:

SEQ ID NO: 15

5′ CCATGTGGTGAGTGGATCTC
FBW7 primer:

SEQ ID NO: 16

3′ CTGCATTCCCAGAGACAAGA
FBW7 probe:

SEQ ID NO: 17

TCCGTGTTTGGGATGTGGAGACA
hRPL19 primer:

SEQ ID NO: 18

5′ AGCGGATTCTCATGGAACA
hRPL19 primer:

SEQ ID NO: 19

3′ CTGGTCAGCCAGGAGCTT
hRPL19 probe:

SEQ ID NO: 20

TCCACAAGCTGAAGGCAGACAAGG
β-TrCP primer:

SEQ ID NO: 21

5′ CATAACTGCTCTGCCAGCTC
β-TrCP primer:

SEQ ID NO: 22

3′ GGTCACTCGGTACCATTCCT
β-TrCP probe:

SEQ ID NO: 23

TGGATGCCAAAT CACTATGTGCTGC
Mcl-1 primer:

SEQ ID NO: 24

5′ GGATGGGTTTGT GGAGTTCT
Mcl-1 primer:

SEQ ID NO: 25

3′ TCCTACTCCAGCAACACCTG
Mcl-1probe:

SEQ ID NO: 26

TGGCATCAGGAATGTG CTGCTG

Real-time RT-PCR analysis was performed using MuLV reverse transcriptase, Amplitaq Gold® kit (Applied Biosystems) and ABI 7500 real time thermal cycler according to the manufacturer's recommendations using at least triplicate samples normalized to hRPL19. Relative levels of FBW7, β-TrCP, and Mcl-1 were calculated following the relative quantitation method provided in the ABI 7500 real-time thermal cycler manual (Applied Biosystems, Life Technologies).

### RNAi Experiments

cIAP1 and cIAP2 siRNA oligos and experiments were performed as described previously (Varfolomeev, E. et al. (2008) The Journal of Biological Chemistry 283:24295-24299). Non-targeting duplex #5 and On Target Plus β-TrCP, sense:

SEQ ID NO: 27

GUGGAAUUUGUGGAACAUCUU

and FBW7 sense:

SEQ ID NO: 28

CCUUCUCUGGAGAGAGAAAUGUU

siRNA oligos were synthesized by Dharmacon and have been previously described (Jin, J. et al. (2003) Genes & development 17:3062-3074; Wei, W., et al (2005) Cancer cell 8:25-33).

For MAPK siRNA experiments, mixes of oligos targeting each isoform were used: Smartpool siRNA oligos for:

ERK1

SEQ ID NO: 29

GACCGGAUGUUAACCUUUA

SEQ ID NO: 30

CCUGCGACCUUAAGAUUUG

SEQ ID NO: 31

CCAAUAAACGGAUCACAGU

SEQ ID NO: 32

AGACUGACCUGUACAAGUU
ERK2

SEQ ID NO: 33

UCGAGUAGCUAUCAAGAAA

SEQ ID NO: 34

CACCAACCAUCGAGCAAAU

SEQ ID NO: 35

GGUGUGCUCUGCUUAUGAU

SEQ ID NO: 36

ACACCAACCUCUCGUACAU

OnTargetPlus set of 4 oligos were synthesized by Dharmacon for:

MAPK8/JNK1

SEQ ID NO: 37

GCCCAGUAAUAUAGUAGUA

SEQ ID NO: 38

GGCAUGGGCUACAAGGAAA

SEQ ID NO: 39

GAAUAGUAUGCGCAGCUUA

SEQ ID NO: 40

GAUGACGCCUUAUGUAGUG
MAPK9/JNK2

SEQ ID NO: 41

GAUUGUUUGUGCUGCAUUU

SEQ ID NO: 42

GGCUGUCGAUGAUAGGUUA

SEQ ID NO: 43

AGCCAACUGUGAGGAAUUA

SEQ ID NO: 44

UCGUGAACUUGUCCUCUUA
MAPK10/JNK3

SEQ ID NO: 45

CAUAUGUGGUGACACGUUA

SEQ ID NO: 46

GGACGACGCCUUACAGCAU

SEQ ID NO: 47

GGAAUUAGACCAUGAGCGA

SEQ ID NO: 48

GGAAAGAACUUAUCUACAA
MAPK11/p38-β

SEQ ID NO: 49

CGACGAGCACGUUCAAUUC

SEQ ID NO: 50

CCAUAGACCUCCUUGGAAG

SEQ ID NO: 51

GCCCUGAGGUUCUGGCAAA

SEQ ID NO: 52

ACGUUCAAUUCCUGGUUUA
MAPK12/p38-γ

SEQ ID NO: 53

GAAGCGUGUUACUUACAAA

SEQ ID NO: 54

GCGCUAAGGUGGCCAUCAA

SEQ ID NO: 55

GCAAGACGCUGUUCAAGGG

SEQ ID NO: 56

GGAGACGCCUCUGUGAAGA
MAPK13/p38-δ

SEQ ID NO: 57

UCAAAGGCCUUAAGUACAU

SEQ ID NO: 58

GCCGUUUGAUGAUUCCUUA

SEQ ID NO: 59

GCUCAAAGGCCUUAAGUAC

SEQ ID NO: 60

GGAGUGGCAUGAAGCUGUA
MAPK14/p38-α

SEQ ID NO: 61

CAAGGUCUCUGGAGGAAUU

SEQ ID NO: 62

GUCAGAAGCUUACAGAUGA

SEQ ID NO: 63

GUCCAUCAUUCAUGCGAAA

SEQ ID NO: 64

CUACAGAGAACUGCGGUUA
SignalSilence GSK-3α/β
siRNA oligos #6301

SEQ ID NO: 65

GAUCUGGAGCUCUCGGUUCU

were synthesized by Cell Signaling Technology and a mix of MULE siGenome siRNA oligos-01 and -04 were synthesized by Dharmacon:

SEQ ID NO: 66

GCAAAGAAAUGGAUAUCAA

SEQ ID NO: 67

GGAAGAGGCUAAAUGUCUA

Transfections were performed as described (Wertz, I. E. et al. (2004) Science (New York, N.Y 303:1371-1374).

Cdc20 siRNA duplex 1 oligos sense:

SEQ ID NO: 68

CGAAAUGACUAUUACCUGAtt
antisense:

SEQ ID NO: 69

UCAGGUAAUAGUCAUUUCGga

were synthesized by Ambion and experiments were performed as described (Huang, H. C., et al (2009) Cancer cell 16:347-358). For viability experiments using stable cell lines transfected with doxycycline-inducible shRNAs to LacZ or Mcl-1 ORF, cells were plated in 10 cm2 plates with 0.2 μg/mL doxycycline for two days. On the third day, cells were plated in to 96-well plates at 5×10³ per well for viability assays as described above. Stable cell lines expressing Mcl-1 phosphomutants plus doxycycline-inducible shRNA targeted to Mcl-1 3′ UTR (sequence in “Plasmids and reagents” section described above) were treated 7 days total with doxycycline to knock down endogenous Mcl-1 expression and simultaneously synchronized and arrested in mitosis as described above. For western blot analysis, cells were harvested at indicated time points and processed as described above.

### Ubiquitination Assays

Cellular ubiquitination assays were performed by synchronizing cells and adding 25 μM MG132 prior to collection as detailed above at the indicated time points. Cells were lysed in CFEB+6 M urea to dissociate non-covalently bound proteins and lysates were diluted 15-fold in CFEB containing 10 mM N-ethyl maleimide, phosphatase inhibitor cocktails 1 and 2 (Sigma), 10 mM NaF, and protease and inhibitor tablets (Roche). Proteins were immunoprecipitated and immunoblotted with the indicated antibodies as outlined above. In vitro ubiquitination assays were performed in 50 μL reaction volumes. FLAG-Mcl-1 was immunoprecipitated from mitotic HeLa cell extracts and purified by FLAG peptide elution as described (Wertz, I. E. et al. (2004) Science (New York, N.Y 303:1371-1374) with phosphatase inhibitor cocktails 1 and 2 added to all steps. HA-CUL1 and HA-DN-CUL1 were expressed in HEK293T cells and purified by HA peptide elution (Covance) following standard protocols. Myc-tagged F-box proteins were prepared by in vitro transcription/translation reactions (High Yield SP6 kit, Promega) and immunoprecipitated with 20 μL 9E10 anti-myc agarose (Roche) in 1 mL CFEB+protease inhibitor tablets, 25 μM MG132, and phosphatase inhibitor cocktails 1 and 2 (Sigma) for 3h at 4° C. Immunocomplexes were washed 3× with CFEB and bound to peptide elution-purified FLAG-Mc1-1 and HA-CUL1 or HA-DN-CUL1 as indicated for 1h at 4° C. with agitation. Subsequently 2 μg N-terminal biotinylated ubiquitin (Boston Biochem), 0.11 μg human recombinant E1 (Boston Biochem), 1 μg UBCHSA (Boston Biochem), phosphatase inhibitor cocktails 1 and 2 (Sigma), and 10× reaction buffer as described previously 25 were combined as indicated and incubated at 30° C. for 2h at 1000 rpm. Reactions were denatured in 6M urea for 20 minutes at room temperature and diluted to 1.25 mL in CFEB+protease inhibitor tablets, 25 μM MG132, and phosphatase inhibitor cocktails 1 and 2 (Sigma) and immunoprecipitated with 25 μL anti-FLAG agarose for 4h at 4° C. The supernatant was divided into 2×625 μL and immunoprecipitated with 25 μL HA- or myc-agarose to assess the amount of HA-CUL1 complex or myc-F-box protein input for each reaction. The immunoprecipitates were washed 3×1 mL CFEB and reduced and alkylated as described above, transferred to membranes, and blotted with the indicated antibodies.

### Pulse-Chase Studies

Wild-type and FBW7−/− HCT116 and DLD1 cells were synchronized and released in to Taxol as described above. Cells were washed and cultured for 60 min at 37° C. in Methionine- and Cysteine-free medium supplemented with 10% diafiltered, heat inactivated FBS (Sigma). Cells were pulsed with 250 μCi 35S Cys/Met—Protein Labeling Mix (Perkin Elmer) for one hour, then washed 3× with PBS and incubated in regular growth medium until collection at the indicated time points. Cells were washed 2× with PBS and lysed using PBS/TDS buffer (1% Tween-20, 0.5% deoxycholate, 0.1% SDS) containing 1 mM NaF with protease inhibitor cocktail tablets (Boehringer Mannheim) and were stored at −20° C. until all timepoints were collected. Lysates were passed through a 25-gauge needle and supernatants were cleared by centrifugation for 10 minutes at 12,500 rpm. Lysates were precleared with non-specific polyclonal antibody and protein A/G beads (Pierce). Precleared lysates were incubated overnight with Mcl-1 antibody and immunocomplexes were captured with Protein A/G beads. Immunocomplexes were separated using 10% SDS-PAGE gels, transferred on to a PVDF membrane, and exposed to film at 4° C.

### Identification of Mitotic Phosphorylation Sites on Mcl-1

FLAG-Mcl-1 was immunoprecipitated from synchronized HCT116 cells arrested in mitosis by paclitaxel and purified by FLAG peptide elution as described above with phosphatase inhibitor cocktails 1 and 2 added to all steps. Elutions were concentrated and subsequently reduced as described above and alkylated (0.176 M n-isopropyl iodoacetamide) at room temperature for 20 minutes. Samples were then separated on a 10% SDS-PAGE gel, and the gel was rinsed briefly in water and stained overnight in Coomasie Brilliant Blue stain containing 50% methanol, followed by destaining in 50% methanol. Gel bands from 45 kDa to 55 kDa (the Mcl-1 migration region) were excised, washed in 50 mM ammonium bicarbonate (Sigma, St Louis, Mo.) containing 5% acetonitrile (Burdick and Jackson, Muskegon, Mich.) for 20 minutes followed by washing in 50 mM ammonium bicarbonate in 50:50 acetonitrile: water for 20 minutes. Gel pieces were dehydrated with acetonitrile and digested with trypsin (Promega, Madison, Wis.), chymotrypsin, or endoproteinase Glu-C (Roche, Nutley, N.J.) in 50 mM ammonium bicarbonate, pH 8.0, overnight at 37° C. Double digestions of trypsin followed by chymotrypsin or endoproteinase Glu-C were also performed. Peptides were extracted from the gel slices in 50 μl of 50:50 v/v acetonitrile: 1% formic acid (Sigma, St. Louis, Mo.) for 30 min followed by 50 μl of pure acetonitrile. Extractions were pooled and evaporated to near dryness, and 7 μl of 0.1% formic acid was subsequently added to samples. Samples were injected via an auto-sampler onto a 75 μM×100 mm column (BEH, 1.7 μM, Waters Corp, Milford, Mass.) at a flow rate of 1 μL/min using a NanoAcquity® UPLC (Waters Corp, Milford, Mass.). A gradient from 98% Solvent A (water+0.1% formic acid) to 80% Solvent B (acetonitrile+0.08% formic acid) was applied over 40 min. Samples were analyzed on-line via nanospray ionization into a hybrid LTQ-Orbitrap® mass spectrometer (Thermo, San Jose, Calif.). Data were collected in data dependent mode with the parent ion being analyzed in the FTMS and the top 8 most abundant ions being selected for fragmentation and analysis in the LTQ, or by targeted analysis. Tandem mass spectrometric data was analyzed using the search algorithms Mascot® (Matrix Sciences, London, UK) or Sequest® (Thermo, San Jose, Calif.). Phosphorylation sites were localized by de novo interpretation and with Ascore® (Harvard University, Cambridge, Mass.) as described (Beausoleil, S. A., et al (2006) Nature biotechnology 24:1285-1292). ¹³C, ¹⁵N labeled peptides representing residues 137-176 of human Mcl-1 were synthesized by Cell Signaling Technologies (Danvers, Mass.). A doubly phosphorylated peptide (S159/T163):

SEQ ID NO: 70

RPAVLPLLELVGESGNNTSTDGpSLPSpTPPPAEEEEDEL

(7.0171)YR, MH+ 4446.0386, was utilized to identify the corresponding peptide in FLAG-Mcl-1 purified from mitotic extracts.

### Recombinant FBW7 Expression and Purification

C-terminal FLAG tagged FBW7 (N2-K707) was cloned into a pAcGP67 vector and expressed in SF9 cells. The protein was purified from the intracellular fraction using ANTI-FLAG M2 Affinity Gel (Sigma) and eluted with 20 mM Tris, pH 8.0, 0.5M NaCl, 10% glycerol, 1 mM EDTA containing 100 μg/ml 3× FLAG PEPTIDE (Sigma). FBW7 was further purified using size exclusion chromatography (HiPrep 16/60 Sephacryl S-300 HR, GE) in storage buffer [20 mM Tris, pH 8.0, 0.5M NaCl, 10% glycerol, 0.5 mM TCEP]. FBW7 concentration was determined using CB X™ Protein Assay (G-Biosciences) and stocks were stored at 4° C.

### Peptide Binding by ELISAs

384-well MaxiSorp® plates (nunc brand, Thermo Fisher Scientific Inc.) were treated for 2 hours with 2.5 mg/mL FBW7 in storage buffer, or storage buffer alone for non-specific binding controls. This incubation and all subsequent steps were conducted at room temperature. Plates were then blocked with 0.5% BSA in TBS [10 mM Tris pH 8, 150 mM sodium chloride] for 2 hours and washed with TBS-T [10 mM Tris pH 8, 150 mM sodium chloride], 0.1% Tween-20]+0.5% BSA. A range of peptide concentrations (0-100 mM) in TBS+0.5% BSA were added to the plates and incubated for 1 hour, then washed with TBS-T+0.5% BSA. Plates were then treated with 125 ng/mL streptavidin-horseradish peroxidase (AMDEX™) in TBS+0.5% BSA for 45 minutes and washed sequentially with TBS-T+0.5% BSA, TBS-T and TBS. Freshly prepared peroxidase substrate was added to the plates for 5 minutes before addition of an equivalent volume of 1M Phosphoric acid stop solution. Plates were read at 450 nm using a Perkin Elmer Victor 3V® plate reader. Signal for each peptide was background corrected by subtracting the appropriate non-specific binding control. The data were then plotted as a function of peptide concentration and fit to a simple, single-site binding equation using Kaleidagraph®, version 3.6 (Synergy Software): θ=([P]T/(Kd+[P]T)), where θ is the fraction of peptide bound, [P]T is the total peptide concentration and Kd is the apparent dissociation constant.

### Recombinant Mcl-1 Protein Production and Purification

For expression and isolation, full length Mcl-1 fused to GST at the N-terminus and a six-histidine tag at the C-terminus was transformed into BL21(DE3) cells. Protein was expressed overnight at 18° C. from cells cultured in terrific broth supplemented with 100 μg/mL carbenicillin. Protein expression was induced by the addition of 0.4 mM IPTG. Cells were harvested by centrifugation and frozen at −20° C. for long-term storage. For protein purification, cells were resuspended 1:10 in buffer (20 mM Phosphate, 50 mM Tris pH 7.5 300 mM NaCl, 5% glycerol) supplemented with 1 mM EDTA, 5 mM DTT, 2% Triton X-100 and protease inhibitor tablets (Roche Diagnostics, Indianapolis, Ind.). Cells were lysed by cell disruption using a microfluidizer (Microfluidics Inc. Newton Mass.) and cell debris removed by centrifugation at 125000 g for 1 hr. The lysate supernatant was decanted over a pre-equilibrated glutathione Sepharose® column. The column was then washed with 20 column volumes of buffer with 5 mM DTT and 0.5% CHAPS. The protein was eluted with 15 mM reduced glutathione. All steps for primary purification were performed at 4° C. For secondary purification protein was further purified by Ni-IMAC and sized exclusion chromatography over an S75 column. TCEP at 1 mM was used in place of DTT for IMAC chromatography.

### In Vitro Kinase Reactions

To determine the suitability of residues in Mcl-1 as kinase substrates, 10 μM of Mcl-1 was incubated with selected kinase at enzyme concentrations between 25 and 100 nM. For these reactions the Mcl-1 was dialyzed into 20 mM Phosphate, 50 mM Tris pH 7.5 150 mM NaCl, 5 mM DTT and 0.5% CHAPS. The protein solution was further supplemented with MgCl₂ to 10 mM and ATP to 1 mM prior to addition of kinase. Purified recombinant kinases were purchased from Invitrogen Co. (Carlsbad, Calif.).

Analysis of Mcl-1 Phosphorylation after Kinase Treatment

10 μl of each of the Mcl-1 kinase reactions (100 pmol) were loaded onto a 4-12% Bis-Tris gel for separation by SDS-PAGE after reduction. Mcl-1 bands were excised from the gel, dehydrated (50% acetonitrile in 50 mM ammonium bicarbonate then 100% acetonitrile washes), and incubated with 0.2 μg trypsin overnight at 37° C. Peptides were eluted from the gel using 50% acetonitrile/1% formic acid, dried in a SpeedVac® (Thermo Fisher Savant), reconstituted in 0.1% formic acid containing custom Mcl-1 isotopically labeled synthetic peptides representing tryptic peptides 105-136 and 137-176 (Cell Signaling Technologies, Danvers, Mass.), as follows:

| From-To, | | | |
|---|---|---|---|
| phos. | | | SEQ |
| label | Peptide, | | ID |
| site | MH+ | Sequence | NO: |
| 137-176 | 4366.072 | RPAVLPLLELVGESGNNTSTDGsLPSTPPP | 71 |
| S159 | | AEEEEDELYR | |
| 137-176 | 4372.086 | RPAVLPLLELVGESGNNTSTDGSLPStPPP | 72 |
| T163 | | AEEEEDELYR | |
| 137-176 | 4446.039 | RPAVLPLLELVGESGNNTSTDGsLPStPPP | 73 |
| S159, | | AEEEEDELYR | |
| T163 | | | |
| 137-176 | 4286.106 | RPAVLPLLELVGESGNNTSTDGSLPSTPPP | 74 |
| | | AEEEEDELYR | |
| 105-136 | 3406.567 | AAPLEEMEAPAADAIMSPEEELDGYEPEPL | 75 |
| | | GK | |
| 105-136 | 3486.533 | AAPLEEMEAPAADAIMsPEEELDGYEPEPL | 76 |
| S121 | | GK | |

Samples were injected in duplicate via autosampler onto a nanoAcquity® UPLC (Waters, Milford, Mass.) and analyzed on-line via nanospray ionization into an LTQ-Orbitrap® mass spectrometer at a concentration of 300 fmol synthetic peptide mix per injection. Areas were integrated for the isotopic and kinase phosphorylated peptides, and compared to their non-phosphorylated peptide counterparts to obtain percent phosphorylation values. For phosphorylation analysis of T92, no synthetic peptide was available so peak areas of the phosphorylated peptide covering residues 76-95 was divided by the total occurrence of peptide 76-95 in both phosphorylated and non-phosphorylated forms.

Analysis of Mcl-1/FBW7 Binding after Mcl-1 In Vitro Phosphorylation

Kinase reactions were performed as described above and reacted for 2 hours at room temperature. Reactions were diluted to a final volume of 600 μL in NTEN buffer (20 mM Tris pH 8.0, 100 mM NaCl, 1 mM EDTA, 0.5% NP40) plus PhosStop® phosphatase inhibitors (Roche) and 4 μg of recombinant FLAG-FBW7 was added. Samples were rotated at 4° C. for 14 hours and FLAG-FBW7/Mcl-1 protein complexes were captured with anti-FLAG agarose (Sigma). Immunoprecipitates were washed 6 times with NTEN buffer and prepared for western blot analysis as described above.

### DNA Copy Number Analysis of Ovarian and NSCLC Tumor Samples

DNA Copy number data for human FBW7 and MCL-1 in ovarian cancers were extracted from two public Agilent Human Genome CGH 244A data sets (n=86, 72) from The Cancer Genome Atlas and three data sets generated by Genentech (GEO accession GSE11960, n=5730; GSE23768, n=51; GSE26075, n=52). For NSCLC, tumor samples from Genentech's internal collections were surveyed using either the Affymetrix Mapping 100K array or the Agilent Human Genome CGH 244A array. All raw data were processed with the Genentech internal data analysis pipeline. For the Affymetrix Mapping 500K and Mapping 100K array data, array intensity signal CEL files were first processed by dChip using the PM/MM difference model and invariant set normalization, and normalized with data for normal samples (Affymetrix). Agilent CGH array data were first processed by Feature Extraction™ Software from Agilent. All processed copy numbers were then centered to a median of 2 and segmented. Copy number values for specific genes were calculated as the mean copy number value for the probe sets bounding the gene location and all intervening probe sets using the segmented data.

### Supplementary Tables 1A-1D

Percent phosphorylation of full-length recombinant Mcl-1 by selected kinases in vitro

### Supplementary Tables 1A-1D

Percent Phosphorylation of Full-Length Recombinant Mcl-1 by Selected Kinases In Vitro

TABLE 1A

| | | SINGLE | | | | CDK1 + KINASE | | | |
|---|---|---|---|---|---|---|---|---|---|
| S121 | | KINASE REACTIONS | | | | PANEL REACTIONS | | | |
| Kinase | Alt. Name | INJ1 | INJ2 | AVE | diff/2 | INJ1 | INJ2 | AVE | diff/2 |
| CDK1 | CDC2 | 0.93 | 0.64 | 0.78 | −0.14 | 1.60 | 1.36 | 1.48 | −0.12 |
| CSNK2 | CKII | 10.20 | 10.36 | 10.28 | 0.08 | 20.17 | 22.78 | 21.48 | −1.31 |
| MAPK8 | JNK1 | 17.66 | 25.46 | 21.56 | 3.90 | 68.97 | 71.14 | 70.06 | 1.09 |
| MAPK9 | JNK2 | 6.46 | 5.57 | 6.02 | −0.45 | 11.75 | 11.89 | 11.82 | −0.07 |
| MAPK10 | JNK3 | 11.61 | 14.67 | 13.14 | 1.53 | 32.82 | 32.88 | 32.85 | 0.03 |
| MAPK11 | p38-β | 5.84 | 5.48 | 5.66 | −0.18 | 10.45 | 9.76 | 10.11 | −0.35 |
| MAPK12 | p38-γ | 10.71 | 11.28 | 11.00 | 0.28 | 18.73 | 17.51 | 18.12 | −0.61 |
| MAPK13 | p38-δ | 10.06 | 6.35 | 8.21 | −1.86 | 20.33 | 20.26 | 20.30 | −0.03 |
| MAPK14 | p38-α | 7.22 | 3.29 | 5.26 | −1.97 | 21.26 | 19.90 | 20.58 | −0.68 |
| No ENZ | N/A | 0.05 | 0.00 | 0.03 | −0.03 | 1.45 | 0.09 | 0.77 | 0.68 |

TABLE 16

| T163 | | SINGLE KINASE REACTIONS | | | |
|---|---|---|---|---|---|
| Kinase | Alt. Name | INJ1 | INJ2 | AVE | diff/2 |
| CDK1 | CDC2 | 29.31 | 28.55 | 28.93 | −0.38 |
| CSNK2 | CKII | 0.00 | 0.00 | 0.00 | 0.00 |
| MAPK8 | JNK1 | 60.42 | 53.11 | 56.77 | −3.66 |
| MAPK9 | JNK2 | 48.92 | 48.11 | 48.52 | 0.41 |
| MAPK10 | JNK3 | 45.09 | 42.70 | 43.90 | −1.20 |
| MAPK11 | p38-β | 53.70 | 50.04 | 51.87 | −1.83 |
| MAPK12 | p38-γ | 55.05 | 51.63 | 53.34 | −1.71 |
| MAPK13 | p38-δ | 65.53 | 64.03 | 64.78 | −0.75 |
| MAPK14 | p38-α | 79.44 | 72.96 | 76.20 | −3.24 |
| No ENZ | N/A | 2.95 | 5.26 | 4.11 | −1.16 |

TABLE 1C

| | | SINGLE | | | | CDK1 + KINASE | | | |
|---|---|---|---|---|---|---|---|---|---|
| S159/T163 | | KINASE REACTIONS | | | | PANEL REACTIONS | | | |
| Kinase | Alt. Name | INJ1 | INJ2 | AVE | diff/2 | INJ1 | INJ2 | AVE | diff/2 |
| CDK1 | CDC2 | 0.00 | 0.29 | 0.15 | 0.15 | 0.00 | 0.20 | 0.10 | 0.10 |
| CSNK2† | CKII S159 | 40.30 | 37.95 | 39.13 | −1.18 | 16.35 | 17.90 | 17.13 | 0.77 |
| CSNK2 | CKII | 5.61 | 1.99 | 3.80 | 1.81 | 8.30 | 7.09 | 7.70 | 0.61 |
| MAPK8 | JNK1 | 15.54 | 18.42 | 16.98 | 1.44 | 10.13 | 8.76 | 9.45 | −0.69 |
| MAPK9 | JNK2 | 5.41 | 4.22 | 4.82 | 0.60 | 0.73 | 0.49 | 0.61 | 0.12 |
| MAPK10 | JNK3 | 16.73 | 16.92 | 16.83 | 0.10 | 3.86 | 4.33 | 4.10 | 0.24 |
| MAPK11 | p38-β | 3.06 | 2.06 | 2.56 | −0.50 | 1.15 | 0.73 | 0.94 | −0.21 |
| MAPK12 | p38-γ | 0.00 | 0.00 | 0.00 | 0.00 | 0.00 | 0.00 | 0.00 | 0.00 |
| MAPK13 | p38-δ | 5.77 | 3.62 | 4.70 | −1.08 | 0.74 | 0.38 | 0.56 | −0.18 |
| MAPK14 | p38-α | 1.12 | 7.57 | 4.35 | 3.23 | 1.84 | 2.43 | 2.14 | 0.30 |
| No ENZ | N/A | 0.00 | 1.54 | 0.77 | −0.77 | 0.87 | 0.27 | 0.57 | 0.30 |

TABLE 1D

| T92* | | SINGLE KINASE REACTIONS | | | |
|---|---|---|---|---|---|
| Kinase | Alt Name | INJ1 | INJ2 | AVE | diff/2 |
| CDK1 | CDC2 | 74.33 | 71.97 | 73.15 | 1.18 |
| CSNK2 | CKII | 0.09 | 0.10 | 0.10 | 0.00 |
| MAPK8 | JNK1 | 5.99 | 6.08 | 6.04 | −0.04 |
| MAPK9 | JNK2 | 2.30 | 1.74 | 2.02 | 0.28 |
| MAPK10 | JNK3 | 10.29 | 8.41 | 9.35 | 0.94 |
| MAPK11 | p38-β | 2.59 | 3.95 | 3.27 | −0.68 |
| MAPK12 | p38-γ | 71.57 | 67.59 | 69.58 | 1.99 |
| MAPK13 | p38-δ | 22.96 | 22.54 | 22.75 | 0.21 |
| MAPK14 | p38-α | 9.67 | 8.20 | 8.94 | 0.74 |
| No ENZ | N/A | 0.02 | 0.00 | 0.01 | 0.01 |

INJ1/2: sample injection 1 or 2; AVE: average value of INJ1 and INJ2; diff/2=(INJ1−INJ2)/2
†CSNK2 in italics indicates the % phos on S159 alone; all other values in Table 1C are % phos on S159+T163
*T92% phos determined using peak areas of 0 and 1P, triple charged state.

### Preparation of Antibody-Drug Conjugates

The anti-tubulin antibody-drug conjugates (ADC) of Formula I may be prepared by several routes, employing organic chemistry reactions, conditions, and reagents known to those skilled in the art, including: (1) reaction of a cysteine group of an antibody with a linker reagent, to form antibody-linker intermediate Ab-L, via a covalent bond, followed by reaction with an activated drug moiety D; and (2) reaction of a nucleophilic group of a drug moiety with a linker reagent, to form drug-linker intermediate D-L, via a covalent bond, followed by reaction with a cysteine group of an antibody, including cysteine-engineered antibodies (Junutula, J. R. et al (2008) Nat. Biotechnol. 26:925-932; Junutula, J. R. (2010) Clin. Cancer Res. 16:4760-4778). Conjugation methods (1) and (2) may be employed with a variety of antibodies, drug moieties, and linkers to prepare the antibody-drug conjugates of Formula I (Lyon, R. et al (2012) Methods in Enzym. 502:123-138; Chari, R. V. (2008) Acc. Chem. Res. 41:98-107; Doronina, et al (2003) Nat. Biotechnol. 21:778-784; Erickson, et al (2010) Bioconj. Chem. 21:84-92; Hamblett et al (2004) Clin. Cancer Res. 10:7063-7070; Lewis Phillips, et al (2008) Cancer Res. 68:9280-9290; McDonagh, et al (2006) Protein Eng. Des. Sel. 19:299-307).

Antibody cysteine thiol groups are nucleophilic and capable of reacting to form covalent bonds with electrophilic groups on linker reagents and drug-linker intermediates including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

Maytansine may, for example, be converted to May-SSCH₃, which can be reduced to the free thiol, May-SH, and reacted with a modified antibody (Chari et al (1992) Cancer Research 52:127-131) to generate a maytansinoid-antibody immunoconjugate with a disulfide linker. Antibody-maytansinoid conjugates with disulfide linkers have been reported (WO 04/016801; U.S. Pat. No. 6,884,874; US 2004/039176 A1; WO 03/068144; US 2004/001838 A1; U.S. Pat. Nos. 6,441,163, 5,208,020, 5,416,064; WO 01/024763). The disulfide linker SPP is constructed with linker reagent N-succinimidyl 4-(2-pyridylthio)pentanoate.

Under certain conditions, the cysteine engineered antibodies may be made reactive for conjugation with linker reagents by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, Mass.). Full length, cysteine engineered monoclonal antibodies (ThioMabs) expressed in CHO cells were reduced with about a 50 fold excess of TCEP for 3 hrs at 37° C. to reduce disulfide bonds which may form between the newly introduced cysteine residues and the cysteine present in the culture media. The reduced ThioMab was diluted and loaded onto HiTrap® S column (GE Healthcare Lifesciences) in 10 mM sodium acetate, pH 5, and eluted with PBS containing 0.3M sodium chloride. Disulfide bonds were reestablished between cysteine residues present in the parent Mab with dilute (200 nM) aqueous copper sulfate (CuSO₄) at room temperature, overnight. Other oxidants, i.e. oxidizing agents, and oxidizing conditions, which are known in the art may be used. Ambient air oxidation is also effective. This mild, partial reoxidation step forms intrachain disulfides efficiently with high fidelity. An approximate 10 fold excess of drug-linker intermediate, e.g. BM(PEO)₄-DM1 was added, mixed, and let stand for about an hour at room temperature to effect conjugation and form the ThioMab antibody-drug conjugate. The conjugation mixture was gel filtered and loaded and eluted through a HiTrap® S column to remove excess drug-linker intermediate and other impurities. Cysteine adducts, presumably along with various interchain disulfide bonds, are reductively cleaved to give a reduced form of the antibody. The interchain disulfide bonds between paired cysteine residues are reformed under partial oxidation conditions, such as exposure to ambient oxygen. The newly introduced, engineered, and unpaired cysteine residues remain available for reaction with linker reagents or drug-linker intermediates to form the antibody conjugates of the invention. The cysteine-engineered antibodies (ThioMabs) expressed in mammalian cell lines result in externally conjugated Cys adduct to an engineered Cys through —S—S— bond formation. Hence the purified ThioMabs have to be treated with reduction and oxidation procedures to produce reactive ThioMabs. These ThioMabs are used to conjugate with maleimide containing cytotoxic anti-tubulin drugs.

Antibody-drug conjugates may be analyzed and purified by reverse-phase and size-exclusion chromatography techniques, and detected by mass spectrometry (Lazar et al (2005) Rapid Commun. Mass Spectrom. 19:1806-1814; Fleming et al (2005) Anal. Biochem. 340:272-278).

The following References are incorporated by reference in their entirety:
1. Jackson, J. R., Patrick, D. R., Dar, M. M. & Huang, P. S. Targeted anti-mitotic therapies: can we improve on tubulin agents? Nature reviews 7, 107-117 (2007).
2. Rieder, C. L. & Maiato, H. Stuck in division or passing through: what happens when cells cannot satisfy the spindle assembly checkpoint. Developmental cell 7, 637-651 (2004).
3. Youle, R. J. & Strasser, A. The BCL-2 protein family: opposing activities that mediate cell death. Nat Rev Mol Cell Biol 9, 47-59 (2008).
4. Willis, S. N. et al. Apoptosis initiated when BH3 ligands engage multiple Bcl-2 homologs, not Bax or Bak. Science (New York, N. Y 315, 856-859 (2007).
5. Varfolomeev, E. & Vucic, D. (Un)expected roles of c-IAPB in apoptotic and NFkappaB signaling pathways. Cell cycle (Georgetown, Tex. 7, 1511-1521 (2008).
6. King, R. W. et al. A 20S complex containing CDC27 and CDC16 catalyzes the mitosis-specific conjugation of ubiquitin to cyclin B. Cell 81, 279-288 (1995).
7. Finley, D. Recognition and processing of ubiquitin-protein conjugates by the proteasome. Annual review of biochemistry 78, 477-513 (2009).
8. Frescas, D. & Pagano, M. Deregulated proteolysis by the F-box proteins SKP2 and beta-TrCP: tipping the scales of cancer. Nature reviews 8, 438-449 (2008).
9. Welcker, M. & Clurman, B. E. FBW7 ubiquitin ligase: a tumour suppressor at the crossroads of cell division, growth and differentiation. Nature reviews 8, 83-93 (2008).
10. Deshaies, R. J. & Joazeiro, C. A. RING domain E3 ubiquitin ligases. Annual review of biochemistry 78, 399-434 (2009).
11. Zhong, Q., Gao, W., Du, F. & Wang, X. Mule/ARF-BP1, a BH3-only E3 ubiquitin ligase, catalyzes the polyubiquitination of Mcl-1 and regulates apoptosis. Cell 121, 1085-1095 (2005).
12. Potapova, T. A. et al. The reversibility of mitotic exit in vertebrate cells. Nature 440, 954-958 (2006).
13. Huang, H. C., Shi, J., Orth, J. D. & Mitchison, T. J. Evidence that mitotic exit is a better cancer therapeutic target than spindle assembly. Cancer cell 16, 347-358 (2009).
14. Domina, A. M., Vrana, J. A., Gregory, M. A., Hann, S. R. & Craig, R. W. MCL1 is phosphorylated in the PEST region and stabilized upon ERK activation in viable cells, and at additional sites with cytotoxic okadaic acid or taxol. Oncogene 23, 5301-5315 (2004).
15. Gascoigne, K. E. & Taylor, S. S. Cancer cells display profound intra- and interline variation following prolonged exposure to antimitotic drugs. Cancer cell 14, 111-122 (2008).
16. Finkin, S., Aylon, Y., Anzi, S., Oren, M. & Shaulian, E. Fbw7 regulates the activity of endoreduplication mediators and the p53 pathway to prevent drug-induced polyploidy. Oncogene 27, 4411-4421 (2008).
17. Terrano, D. T., Upreti, M. & Chambers, T. C. Cyclin-dependent kinase 1-mediated Bcl-xL/Bcl-2 phosphorylation acts as a functional link coupling mitotic arrest and apoptosis. Molecular and cellular biology 30, 640-656 (2010).
18. Ozalp, S. S., Yalcin, O. T., Tanir, M., Kabukcuoglu, S. & Etiz, E. Multidrug resistance gene-1 (Pgp) expression in epithelial ovarian malignancies. European journal of gynaecological oncology 23, 337-340 (2002).
19. Mesquita, B. et al. No significant role for beta tubulin mutations and mismatch repair defects in ovarian cancer resistance to paclitaxel/cisplatin. BMC cancer 5, 101 (2005).
20. Chen, L. et al. Differential targeting of prosurvival Bcl-2 proteins by their BH3-only ligands allows complementary apoptotic function. Molecular cell 17, 393-403 (2005).
21. Gray, D. C. et al. pHUSH: a single vector system for conditional gene expression. BMC biotechnology 7, 61 (2007).
22. Kitamura, T. et al. Retrovirus-mediated gene transfer and expression cloning: powerful tools in functional genomics. Experimental hematology 31, 1007-1014 (2003).
23. Vince, J. E. et al. IAP antagonists target cIAP1 to induce TNFalpha-dependent apoptosis. Cell 131, 682-693 (2007).
24. Ekert, P. G. et al. Apaf-1 and caspase-9 accelerate apoptosis, but do not determine whether factor-deprived or drug-treated cells die. The Journal of cell biology 165, 835-842 (2004).
25. Wertz, I. E. et al. Human De-etiolated-1 regulates c-Jun by assembling a CUL4A ubiquitin ligase. Science (New York, N. Y 303, 1371-1374 (2004).
26. Varfolomeev, E. et al. c-IAP1 and c-IAP2 are critical mediators of tumor necrosis factor alpha (TNFalpha)-induced NF-kappaB activation. The Journal of biological chemistry 283, 24295-24299 (2008).
27. Jin, J. et al. SCFbeta-TRCP links Chk1 signaling to degradation of the Cdc25A protein phosphatase. Genes & development 17, 3062-3074 (2003).
28. Wei, W., Jin, J., Schlisio, S., Harper, J. W. & Kaelin, W. G., Jr. The v-Jun point mutation allows c-Jun to escape GSK3-dependent recognition and destruction by the Fbw7 ubiquitin ligase. Cancer cell 8, 25-33 (2005).
29. Beausoleil, S. A., Villen, J., Gerber, S. A., Rush, J. & Gygi, S. P. A probability-based approach for high-throughput protein phosphorylation analysis and site localization. Nature biotechnology 24, 1285-1292 (2006).
30. Haverty, P. M., Hon, L. S., Kaminker, J. S., Chant, J. & Zhang, Z. High-resolution analysis of copy number alterations and associated expression changes in ovarian tumors. BMC Med Genomics 2, 21 (2009).
31. Inuzuka et al “SCF^{Fbw7} Regulates Cellular Apoptosis By Targeting Mcl-1 for Ubiquitination and Destruction” (2011) Nature. March 3; 471(7336):104-9.
32. Tan et al, “Navitoclax enhances the efficacy of taxanes in non-small cell lung cancer models” (2011) Clin Cancer Res. March 15; 17(6):1394-404.
33. Harley et al, “Phosphorylation of Mcl-1 by CDK1-cyclin B1 initiates its Cdc20-dependent destruction during mitotic arrest” (2010) EMBO J. July 21; 29(14):2407-20. Epub 2010 Jun. 4).
34. Wertz et al, “Sensitivity of antitubulin chemotherapeutics is regulated by MCL1 and FBW7” (2011) Nature March 3; 471:110-114.

We claim:

1. A method of treating a hyperproliferative disorder in a patient comprising:
administering a therapeutically effective amount of an anti-tubulin chemotherapeutic agent to the patient,
wherein a biological sample obtained from the patient, prior to administration of the anti-tubulin chemotherapeutic agent to the patient, has been tested for Mcl-1 and/or FBW7 status, and
wherein Mcl-1 and/or FBW7 status is indicative of therapeutic responsiveness by the patient to the anti-tubulin chemotherapeutic agent.

2. The method of claim 1 wherein the biological sample has been tested by measuring functional Mcl-1 protein level, wherein an increased level of functional Mcl-1 protein indicates that the patient will be resistant to the anti-tubulin chemotherapeutic agent.

3. The method of claim 1 wherein the biological sample has been tested by measuring functional FBW7 protein level, wherein a decreased level of functional FBW7 protein indicates that the patient will be resistant to the anti-tubulin chemotherapeutic agent.

4. A method of monitoring whether a patient with a hyperproliferative disorder will respond to treatment with an anti-tubulin chemotherapeutic agent, the method comprising:
(a) detecting Mcl-1 and/or FBW7 in a biological sample obtained from the patient following administration of the at least one dose of an anti-tubulin chemotherapeutic agent; and
(b) comparing Mcl-1 and/or FBW7 status in a biological sample obtained from the patient prior to administration of the anti-tubulin chemotherapeutic agent to the patient,
wherein a change or modulation of Mcl-1 and/or FBW7 status in the sample obtained following administration of the anti-tubulin chemotherapeutic agent identifies a patient who will respond to treatment with an anti-tubulin chemotherapeutic agent.

5. A method of optimizing therapeutic efficacy of an anti-tubulin chemotherapeutic agent, the method comprising:
(a) detecting Mcl-1 and/or FBW7 in a biological sample obtained from a patient who has received at least one dose of an anti-tubulin chemotherapeutic agent following administration of the at least one dose of an anti-tubulin chemotherapeutic agent; and
(b) comparing the Mcl-1 and/or FBW7 status in a biological sample obtained from the patient prior to administration of the anti-tubulin chemotherapeutic agent to the patient,
wherein a change or modulation of Mcl-1 and/or FBW7 in the sample obtained following administration of the anti-tubulin chemotherapeutic agent identifies a patient who has an increased likelihood of benefit from treatment with an anti-tubulin chemotherapeutic agent.

6. The method of any one of claims 1 to 5, wherein the change or modulation of Mcl-1 and/or FBW7 is detected by sequencing the genomic DNA or reverse-transcribed PCR products of the biological sample, whereby one or more mutations are detected.

7. The method of any one of claims 1 to 5, wherein the change or modulation of Mcl-1 and/or FBW7 status is detected by gene expression analysis of the biological sample by quantitation of message level or assessment of copy number.

8. The method of any one of claims 1 to 5, wherein the change or modulation of Mcl-1 and/or FBW7 status is detected by analysis of proteins of the biological sample by a method selected from immunohistochemistry, immunocytochemistry, ELISA, and mass spectrometric analysis,
whereby degradation, stabilization, post-translational phosphorylation or post-translational ubiquitination of the proteins is detected.

9. The method of any one of claims 1 to 5, wherein the anti-tubulin chemotherapeutic agent is selected from paclitaxel, docetaxel, vincristine, vinblastine, vinorelbine, eribulin, combretastatin, maytansines, dolastatins, auristatins, and the antibody-drug conjugates thereof.

10. The method of claim 9 wherein the anti-tubulin chemotherapeutic agent is an antibody-drug conjugate compound having Formula I:
Ab-(L-D)ₚ I
comprising an antibody (Ab), and an anti-tubulin drug moiety (D) wherein the antibody has one or more free cysteine amino acids, and the antibody is attached through the one or more free cysteine amino acids by a linker moiety (L) to D and where p is an integer from 1 to about 8.

11. The method of claim 10 wherein the anti-tubulin drug moiety (D) is selected from a maytansinoid and an auristatin.

12. The method of claim 11 wherein the anti-tubulin drug moiety (D) is an auristatin selected from MMAE and MMAF having the structures: 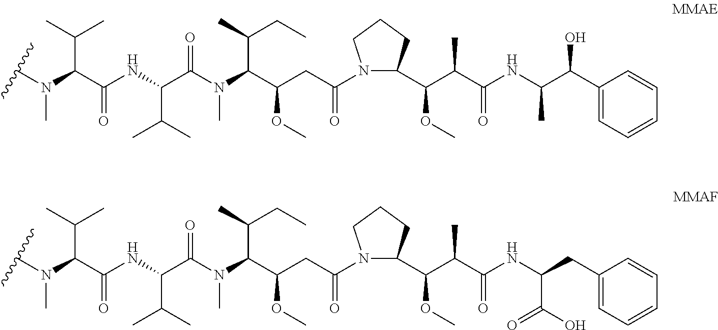 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]
where the wavy line indicates the site of attachment to the linker (L).

13. The method of claim 12 wherein the antibody-drug conjugate compound is selected from the structures: 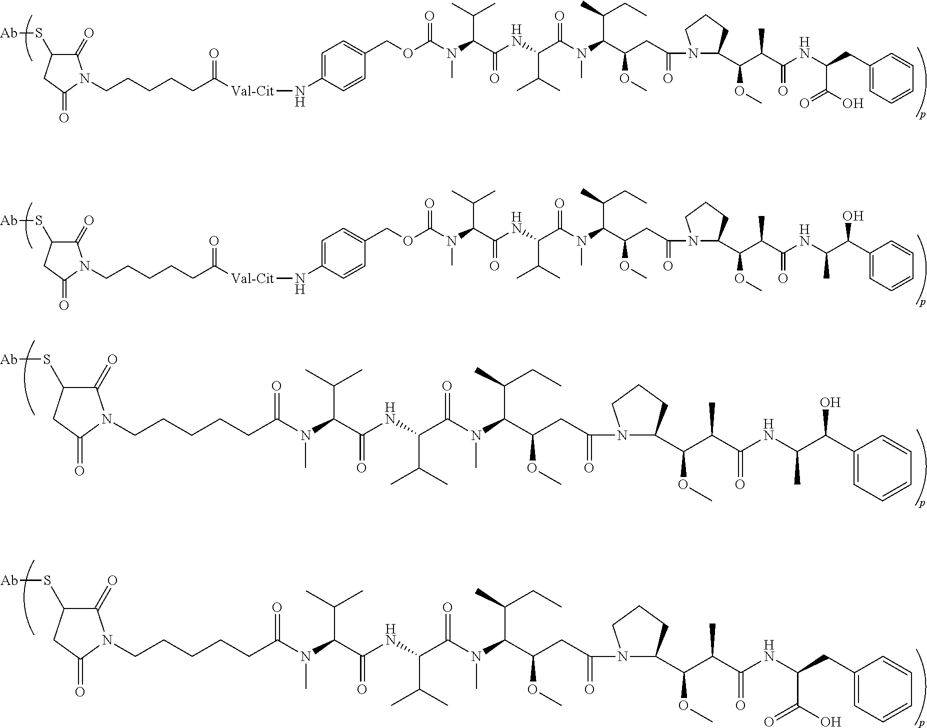
where Val is valine and Cit is citrulline.

14. The method of claim 10 wherein Ab is an antibody that binds to one or more tumor-associated antigens or cell-surface receptors selected from (1)-(36):
(1) BMPR1B;
(2) E16;
(3) STEAP1;
(4) 0772P (MUC16);
(5) MPF (MSLN, mesothelin);
(6) Napi3b;
(7) Sema 5b;
(8) PSCA hlg;
(9) ETBR;
(10) MSG783;
(11) STEAP2;
(12) TrpM4;
(13) CRIPTO;
(14) CD21;
(15) CD79b;
(16) FcRH2;
(17) HER2;
(18) NCA;
(19) MDP;
(20) IL20Rα;
(21) Brevican;
(22) EphB2R;
(23) ASLG659;
(24) PSCA;
(25) GEDA;
(26) BAFF-R;
(27) CD22;
(28) CD79a;
(29) CXCR5;
(30) HLA-DOB;
(31) P2X5;
(32) CD72;
(33) LY64;
(34) FcRH1;
(35) IRTA2 (FcRH5); and
(36) TENB2.

15. The method of claim 1 or 2, wherein the hyperproliferative disorder is cancer selected from squamous cell cancer, lung cancer including small-cell lung cancer, non-small cell lung cancer (NSCLC), adenocarcinoma of the lung and squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, and mesothelioma.

16. The method of claim 1 or 2, wherein the hyperproliferative disorder is a hematological malignancy selected from non-Hodgkin's lymphoma, diffuse large hematopoietic lymphoma, follicular lymphoma, mantle cell lymphoma, chronic lymphocytic leukemia, multiple myeloma, acute myelogenous leukemia, and myeloid cell leukemia.

17. The method of claim 1 wherein a therapeutically effective dosage of an anti-tubulin chemotherapeutic agent is determined and adjusted based upon, inhibition or modulation of Mcl-1 or FBW7.

18. A method of identifying a biomarker for monitoring responsiveness to an anti-tubulin chemotherapeutic agent, the method comprising:
(a) detecting the expression, modulation, or activity of a biomarker in a biological sample obtained from a patient who has received at least one dose of an anti-tubulin chemotherapeutic agent wherein the biomarker is Mcl-1 and/or FBW7; and
(b) comparing the expression, modulation, or activity of the biomarker to the status of the biomarker in a reference sample wherein the reference sample is a biological sample obtained from the patient prior to administration of the anti-tubulin chemotherapeutic agent to the patient;
wherein the modulation of the biomarker changes by at least 2 fold lower compared to the reference sample is identified as a biomarker useful for monitoring responsiveness to an anti-tubulin chemotherapeutic agent.

19. The method of claim 18, wherein the modulation of the biomarker changes by at least 2-fold lower in the biological sample compared to the reference sample is identified as a biomarker useful for monitoring responsiveness to an anti-tubulin chemotherapeutic agent.

20. The method of claim 18 wherein the biomarker is Mcl-1 and modulation of Mcl-1 is an increased level of Mcl-1.

21. The method of claim 18 wherein the biomarker is FBW7 and modulation of FBW7 is a decreased level of FBW7.

22. A method of treating a hyperproliferative disorder in a patient, comprising administering a therapeutically effective amount of an anti-tubulin chemotherapeutic agent the patient, wherein treatment is based upon a sample from the patient having an Mcl-1 or FBW7 mutation.
